SEQUENCE LISTING (1233 sequences)

SEQ ID NO: 1 - protein, 9 aa, Homo sapiens
NRTTHLCSL

SEQ ID NO: 2 - protein, 9 aa, Homo sapiens
THLCSLILL

SEQ ID NO: 3 - protein, 9 aa, Homo sapiens
ILLGMIAQR

SEQ ID NO: 4 - protein, 9 aa, Homo sapiens
MIAQRSFIR

SEQ ID NO: 5 - protein, 9 aa, Homo sapiens
RSFIRGPTQ

SEQ ID NO: 6 - protein, 9 aa, Homo sapiens
GPTQCPLTW

SEQ ID NO: 7 - protein, 9 aa, Homo sapiens
TWCPTRMAW

SEQ ID NO: 8 - protein, 9 aa, Homo sapiens
CPTRMAWMW

SEQ ID NO: 9 - protein, 9 aa, Homo sapiens
QSLENLINF

SEQ ID NO: 10 - protein, 9 aa, Homo sapiens
SLENLINFL

SEQ ID NO: 11 - protein, 9 aa, Homo sapiens
NFLPIGEGR

SEQ ID NO: 12 - protein, 9 aa, Homo sapiens
LPIGEGRSM

SEQ ID NO: 13 - protein, 9 aa, Homo sapiens
GRSMNLHGI

SEQ ID NO: 14 - protein, 9 aa, Homo sapiens
RVLGSCPPR

SEQ ID NO: 15 - protein, 9 aa, Homo sapiens
VLGSCPPRR

SEQ ID NO: 16 - protein, 9 aa, Homo sapiens
PPRRFLPPL

SEQ ID NO: 17 - protein, 9 aa, Homo sapiens
RRFLPPLSA

SEQ ID NO: 18 - protein, 9 aa, Homo sapiens
RFLPPLSAR

SEQ ID NO: 19 - protein, 9 aa, Homo sapiens
AVANRMDQF

SEQ ID NO: 20 - protein, 9 aa, Homo sapiens
ANRMDQFTF

SEQ ID NO: 21 - protein, 9 aa, Homo sapiens
FTFQTDKQR

SEQ ID NO: 22 - protein, 9 aa, Homo sapiens
VMAYGSLDF

SEQ ID NO: 23 - protein, 9 aa, Homo sapiens
MAYGSLDFY

SEQ ID NO: 24 - protein, 9 aa, Homo sapiens
AYGSLDFYI

SEQ ID NO: 25 - protein, 9 aa, Homo sapiens
GSLDFYIFL

SEQ ID NO: 26 - protein, 9 aa, Homo sapiens
YIFLDRCLY

SEQ ID NO: 27 - protein, 9 aa, Homo sapiens
FLDRCLYCY

SEQ ID NO: 28 - protein, 9 aa, Homo sapiens
VIFAALPIL

SEQ ID NO: 29 - protein, 9 aa, Homo sapiens
IFAALPILF

SEQ ID NO: 30 - protein, 9 aa, Homo sapiens
AALPILFQV

SEQ ID NO: 31 - protein, 9 aa, Homo sapiens
LPILFQVPT

SEQ ID NO: 32 - protein, 9 aa, Homo sapiens
HRLPFWLKS

SEQ ID NO: 33 - protein, 9 aa, Homo sapiens
RLPFWLKSV

SEQ ID NO: 34 - protein, 9 aa, Homo sapiens
LPFWLKSVL

SEQ ID NO: 35 - protein, 9 aa, Homo sapiens
SRKNCGTWL

SEQ ID NO: 36 - protein, 9 aa, Homo sapiens
GTWLMVLER

SEQ ID NO: 37 - protein, 9 aa, Homo sapiens
KKYKVEYVL

SEQ ID NO: 38 - protein, 9 aa, Homo sapiens
DSILSCTAR

SEQ ID NO: 39 - protein, 9 aa, Homo sapiens
RTLKMGTSS

SEQ ID NO: 40 - protein, 9 aa, Homo sapiens
KMGTSSTAF

SEQ ID NO: 41 - protein, 9 aa, Homo sapiens
STAFSEPTR

SEQ ID NO: 42 - protein, 9 aa, Homo sapiens
AFSEPTRTR

SEQ ID NO: 43 - protein, 9 aa, Homo sapiens
FSEPTRTRW

SEQ ID NO: 44 - protein, 9 aa, Homo sapiens
RWKCILPSY

SEQ ID NO: 45 - protein, 9 aa, Homo sapiens
AYKDFLWCF

SEQ ID NO: 46 - protein, 9 aa, Homo sapiens
FLWCFPFSL

SEQ ID NO: 47 - protein, 9 aa, Homo sapiens
LELFLSRAL

SEQ ID NO: 48 - protein, 9 aa, Homo sapiens
ELFLSRALR

SEQ ID NO: 49 - protein, 9 aa, Homo sapiens
FLSRALRAL

SEQ ID NO: 50 - protein, 9 aa, Homo sapiens
SRALRALHV

SEQ ID NO: 51 - protein, 9 aa, Homo sapiens
RALRALHVL

SEQ ID NO: 52 - protein, 9 aa, Homo sapiens
ALRALHVLW

SEQ ID NO: 53 - protein, 9 aa, Homo sapiens
HVLWNGFQL

SEQ ID NO: 54 - protein, 9 aa, Homo sapiens
VLWNGFQLH

SEQ ID NO: 55 - protein, 9 aa, Homo sapiens
ERARDIQPL

SEQ ID NO: 56 - protein, 9 aa, Homo sapiens
RARDIQPLC

SEQ ID NO: 57 - protein, 9 aa, Homo sapiens
DIQPLCPCR

SEQ ID NO: 58 - protein, 9 aa, Homo sapiens
IDQKFVQQV

SEQ ID NO: 59 - protein, 9 aa, Homo sapiens
QKFVQQVSF

SEQ ID NO: 60 - protein, 9 aa, Homo sapiens
KFVQQVSFK

SEQ ID NO: 61 - protein, 9 aa, Homo sapiens
VSFKAPGAF

SEQ ID NO: 62 - protein, 9 aa, Homo sapiens
DMESSPTHR

SEQ ID NO: 63 - protein, 9 aa, Homo sapiens
ELRSKSGHR

SEQ ID NO: 64 - protein, 9 aa, Homo sapiens
LRSKSGHRL

SEQ ID NO: 65 - protein, 9 aa, Homo sapiens
RSKSGHRLF

SEQ ID NO: 66 - protein, 9 aa, Homo sapiens
RLFPISPET

SEQ ID NO: 67 - protein, 9 aa, Homo sapiens
LFPISPETL

SEQ ID NO: 68 - protein, 9 aa, Homo sapiens
FPISPETLH

SEQ ID NO: 69 - protein, 9 aa, Homo sapiens
SPETLHFPV

SEQ ID NO: 70 - protein, 9 aa, Homo sapiens
ETLHFPVSR

SEQ ID NO: 71 - protein, 9 aa, Homo sapiens
LHFPVSRHL

SEQ ID NO: 72 - protein, 9 aa, Homo sapiens
FPVSRHLGS

SEQ ID NO: 73 - protein, 9 aa, Homo sapiens
ARHTERTAL

SEQ ID NO: 74 - protein, 9 aa, Homo sapiens
RHTERTALL

SEQ ID NO: 75 - protein, 9 aa, Homo sapiens
ERTALLPGL

SEQ ID NO: 76 - protein, 9 aa, Homo sapiens
ALLPGLPAA

SEQ ID NO: 77 - protein, 9 aa, Homo sapiens
ASPRRPAGW

SEQ ID NO: 78 - protein, 9 aa, Homo sapiens
WRCENGRLL

SEQ ID NO: 79 - protein, 9 aa, Homo sapiens
MKKLPITYL

SEQ ID NO: 80 - protein, 9 aa, Homo sapiens
KLPITYLSL

SEQ ID NO: 81 - protein, 9 aa, Homo sapiens
LPITYLSLL

SEQ ID NO: 82 - protein, 9 aa, Homo sapiens
ITYLSLLSI

SEQ ID NO: 83 - protein, 9 aa, Homo sapiens
KQGGLPLPL

SEQ ID NO: 84 - protein, 9 aa, Homo sapiens
LPLPLLCGA

SEQ ID NO: 85 - protein, 9 aa, Homo sapiens
RAPGVPDSL

SEQ ID NO: 86 - protein, 9 aa, Homo sapiens
GVPDSLPGA

SEQ ID NO: 87 - protein, 9 aa, Homo sapiens
DSLPGAGAR

SEQ ID NO: 88 - protein, 9 aa, Homo sapiens
RVCCPTSPK

SEQ ID NO: 89 - protein, 9 aa, Homo sapiens
VLFLTLNEV

SEQ ID NO: 90 - protein, 9 aa, Homo sapiens
LFLTLNEVF

SEQ ID NO: 91 - protein, 9 aa, Homo sapiens
FLTLNEVFL

SEQ ID NO: 92 - protein, 9 aa, Homo sapiens
TLNEVFLIL

SEQ ID NO: 93 - protein, 9 aa, Homo sapiens
NEVFLILQV

SEQ ID NO: 94 - protein, 9 aa, Homo sapiens
FLILQVHGT

SEQ ID NO: 95 - protein, 9 aa, Homo sapiens
YPDDGMGSS

SEQ ID NO: 96 - protein, 9 aa, Homo sapiens
RSSLTAHSM

SEQ ID NO: 97 - protein, 9 aa, Homo sapiens
SSLTAHSMR

SEQ ID NO: 98 - protein, 9 aa, Homo sapiens
HSMREIHGI

SEQ ID NO: 99 - protein, 9 aa, Homo sapiens
REIHGIAGT

SEQ ID NO: 100 - protein, 9 aa, Homo sapiens
RALINSVQL

SEQ ID NO: 101 - protein, 9 aa, Homo sapiens
DRLAQSVFL

SEQ ID NO: 102 - protein, 9 aa, Homo sapiens
LAQSVFLSR

SEQ ID NO: 103 - protein, 9 aa, Homo sapiens
QTCSQPAAR

SEQ ID NO: 104 - protein, 9 aa, Homo sapiens
HKILTANSL

SEQ ID NO: 105 - protein, 9 aa, Homo sapiens
LTANSLSSV

SEQ ID NO: 106 - protein, 9 aa, Homo sapiens
TANSLSSVF

SEQ ID NO: 107 - protein, 9 aa, Homo sapiens
SHGHGRIAL

SEQ ID NO: 108 - protein, 9 aa, Homo sapiens
HGHGRIALY

SEQ ID NO: 109 - protein, 9 aa, Homo sapiens
GRIALYSCR

SEQ ID NO: 110 - protein, 9 aa, Homo sapiens
RQAPEAPRR

SEQ ID NO: 111 - protein, 9 aa, Homo sapiens
QEILERQPT

SEQ ID NO: 112 - protein, 9 aa, Homo sapiens
EILERQPTL

SEQ ID NO: 113 - protein, 9 aa, Homo sapiens
ILERQPTLF

SEQ ID NO: 114 - protein, 9 aa, Homo sapiens
LERQPTLFF

SEQ ID NO: 115 - protein, 9 aa, Homo sapiens
YPMDSPEPG

SEQ ID NO: 116 - protein, 9 aa, Homo sapiens
EPGGAGSTL

SEQ ID NO: 117 - protein, 9 aa, Homo sapiens
LEFEDFDVF

SEQ ID NO: 118 - protein, 9 aa, Homo sapiens
EFEDFDVFI

SEQ ID NO: 119 - protein, 9 aa, Homo sapiens
FEDFDVFIF

SEQ ID NO: 120 - protein, 9 aa, Homo sapiens
EDFDVFIFF

SEQ ID NO: 121 - protein, 9 aa, Homo sapiens
DFDVFIFFR

SEQ ID NO: 122 - protein, 9 aa, Homo sapiens
AAAMTRTQL

SEQ ID NO: 123 - protein, 9 aa, Homo sapiens
AAMTRTQLL

SEQ ID NO: 124 - protein, 9 aa, Homo sapiens
MTRTQLLST

SEQ ID NO: 125 - protein, 9 aa, Homo sapiens
RTQLLSTGL

SEQ ID NO: 126 - protein, 9 aa, Homo sapiens
TQLLSTGLL

SEQ ID NO: 127 - protein, 9 aa, Homo sapiens
LLSTGLLQW

SEQ ID NO: 128 - protein, 9 aa, Homo sapiens
DTTAIDVQY

SEQ ID NO: 129 - protein, 9 aa, Homo sapiens
TTAIDVQYL

SEQ ID NO: 130 - protein, 9 aa, Homo sapiens
TAIDVQYLF

SEQ ID NO: 131 - protein, 9 aa, Homo sapiens
AIDVQYLFL

SEQ ID NO: 132 - protein, 9 aa, Homo sapiens
YLFLPPGGA

SEQ ID NO: 133 - protein, 9 aa, Homo sapiens
FLPPGGAPV

SEQ ID NO: 134 - protein, 9 aa, Homo sapiens
EQRLQQEDF

SEQ ID NO: 135 - protein, 9 aa, Homo sapiens
QRLQQEDFL

SEQ ID NO: 136 - protein, 9 aa, Homo sapiens
RLQQEDFLR

SEQ ID NO: 137 - protein, 9 aa, Homo sapiens
LQQEDFLRV

SEQ ID NO: 138 - protein, 9 aa, Homo sapiens
EDFLRVQQL

SEQ ID NO: 139 - protein, 9 aa, Homo sapiens
QQLSAAGPL

SEQ ID NO: 140 - protein, 9 aa, Homo sapiens
QLSAAGPLR

SEQ ID NO: 141 - protein, 9 aa, Homo sapiens
AAGPLRPQL

SEQ ID NO: 142 - protein, 9 aa, Homo sapiens
DGHQPLEQY

SEQ ID NO: 143 - protein, 9 aa, Homo sapiens
QPLEQYPEA

SEQ ID NO: 144 - protein, 9 aa, Homo sapiens
LEQYPEAIL

SEQ ID NO: 145 - protein, 9 aa, Homo sapiens
KSSPSMAPL

SEQ ID NO: 146 - protein, 9 aa, Homo sapiens
SPSMAPLPT

SEQ ID NO: 147 - protein, 9 aa, Homo sapiens
SMAPLPTPR

SEQ ID NO: 148 - protein, 9 aa, Homo sapiens
APLPTPRSC

SEQ ID NO: 149 - protein, 9 aa, Homo sapiens
TSRPLSLSS

SEQ ID NO: 150 - protein, 9 aa, Homo sapiens
SRPLSLSSF

SEQ ID NO: 151 - protein, 9 aa, Homo sapiens
RPLSLSSFW

SEQ ID NO: 152 - protein, 9 aa, Homo sapiens
LSLSSFWAR

SEQ ID NO: 153 - protein, 9 aa, Homo sapiens
SDGSLSDLW

SEQ ID NO: 154 - protein, 9 aa, Homo sapiens
SDGSLSDLW

SEQ ID NO: 155 - protein, 9 aa, Homo sapiens
SLSDLWAPA

SEQ ID NO: 156 - protein, 9 aa, Homo sapiens
SLSDLWAPA

SEQ ID NO: 157 - protein, 9 aa, Homo sapiens
LWAPAGPSI

SEQ ID NO: 158 - protein, 9 aa, Homo sapiens
WAPAGPSIW

SEQ ID NO: 159 - protein, 9 aa, Homo sapiens
LWAPAGPSI

SEQ ID NO: 160 - protein, 9 aa, Homo sapiens
APAGPSIWW

SEQ ID NO: 161 - protein, 9 aa, Homo sapiens
WAPAGPSIW

SEQ ID NO: 162 - protein, 9 aa, Homo sapiens
APAGPSIWW

SEQ ID NO: 163 - protein, 9 aa, Homo sapiens
MEAMLLYLV

SEQ ID NO: 164 - protein, 9 aa, Homo sapiens
KENNKLEDL

SEQ ID NO: 165 - protein, 9 aa, Homo sapiens
LRFSSEENM

SEQ ID NO: 166 - protein, 9 aa, Homo sapiens
SAHCPGSLR

SEQ ID NO: 167 - protein, 9 aa, Homo sapiens
TACVPHRTR

SEQ ID NO: 168 - protein, 9 aa, Homo sapiens
HRTRGSNSI

SEQ ID NO: 169 - protein, 9 aa, Homo sapiens
EERFALVLV

SEQ ID NO: 170 - protein, 9 aa, Homo sapiens
ERFALVLVL

SEQ ID NO: 171 - protein, 9 aa, Homo sapiens
ASRQGPAPA

SEQ ID NO: 172 - protein, 9 aa, Homo sapiens
SRQGPAPAA

SEQ ID NO: 173 - protein, 9 aa, Homo sapiens
RQGPAPAAV

SEQ ID NO: 174 - protein, 9 aa, Homo sapiens
NVLNKSNEV

SEQ ID NO: 175 - protein, 9 aa, Homo sapiens
VLNKSNEVF

SEQ ID NO: 176 - protein, 9 aa, Homo sapiens
NKSNEVFPL

SEQ ID NO: 177 - protein, 9 aa, Homo sapiens
KSNEVFPLI

SEQ ID NO: 178 - protein, 9 aa, Homo sapiens
NEVFPLILA

SEQ ID NO: 179 - protein, 9 aa, Homo sapiens
EVFPLILAS

SEQ ID NO: 180 - protein, 9 aa, Homo sapiens
FPLILASQM

SEQ ID NO: 181 - protein, 9 aa, Homo sapiens
ASQMYVPGK

SEQ ID NO: 182 - protein, 9 aa, Homo sapiens
VSTPSLSSL

SEQ ID NO: 183 - protein, 9 aa, Homo sapiens
TPSLSSLTF

SEQ ID NO: 184 - protein, 9 aa, Homo sapiens
SSLTFLPLY

SEQ ID NO: 185 - protein, 9 aa, Homo sapiens
SLTFLPLYV

SEQ ID NO: 186 - protein, 9 aa, Homo sapiens
FLPLYVIPA

SEQ ID NO: 187 - protein, 9 aa, Homo sapiens
LYVIPARSR

SEQ ID NO: 188 - protein, 9 aa, Homo sapiens
YVIPARSRA

SEQ ID NO: 189 - protein, 9 aa, Homo sapiens
IPLPLIKSS

SEQ ID NO: 190 - protein, 9 aa, Homo sapiens
SSLSPLSEL

SEQ ID NO: 191 - protein, 9 aa, Homo sapiens
SELSVPAAP

SEQ ID NO: 192 - protein, 9 aa, Homo sapiens
VPAAPGPSF

SEQ ID NO: 193 - protein, 9 aa, Homo sapiens
APGPSFLPF

SEQ ID NO: 194 - protein, 9 aa, Homo sapiens
LPFSSGPPP

SEQ ID NO: 195 - protein, 9 aa, Homo sapiens
REHCFSDFL

SEQ ID NO: 196 - protein, 9 aa, Homo sapiens
FSDFLAGSL

SEQ ID NO: 197 - protein, 9 aa, Homo sapiens
DFLAGSLRK

SEQ ID NO: 198 - protein, 9 aa, Homo sapiens
SLRKQMKVL

SEQ ID NO: 199 - protein, 9 aa, Homo sapiens
QMKVLRITK

SEQ ID NO: 200 - protein, 9 aa, Homo sapiens
MKVLRITKM

SEQ ID NO: 201 - protein, 9 aa, Homo sapiens
LRITKMPCF

SEQ ID NO: 202 - protein, 9 aa, Homo sapiens
RITKMPCFR

SEQ ID NO: 203 - protein, 9 aa, Homo sapiens
KFDFSYPGI

SEQ ID NO: 204 - protein, 9 aa, Homo sapiens
FSYPGIATS

SEQ ID NO: 205 - protein, 9 aa, Homo sapiens
YPGIATSSL

SEQ ID NO: 206 - protein, 9 aa, Homo sapiens
FGKPYFIKF

SEQ ID NO: 207 - protein, 9 aa, Homo sapiens
KPYFIKFIF

SEQ ID NO: 208 - protein, 9 aa, Homo sapiens
FIFKSANGI

SEQ ID NO: 209 - protein, 9 aa, Homo sapiens
KSANGICCR

SEQ ID NO: 210 - protein, 9 aa, Homo sapiens
SANGICCRR

SEQ ID NO: 211 - protein, 9 aa, Homo sapiens
RRKKALHCL

SEQ ID NO: 212 - protein, 9 aa, Homo sapiens
STPTPSLHL

SEQ ID NO: 213 - protein, 9 aa, Homo sapiens
KELQGQCFL

SEQ ID NO: 214 - protein, 9 aa, Homo sapiens
HRMWRLWTH

SEQ ID NO: 215 - protein, 9 aa, Homo sapiens
RLWTHEVEH

SEQ ID NO: 216 - protein, 9 aa, Homo sapiens
LWTHEVEHY

SEQ ID NO: 217 - protein, 9 aa, Homo sapiens
HEVEHYCIL

SEQ ID NO: 218 - protein, 9 aa, Homo sapiens
VEHYCILLF

SEQ ID NO: 219 - protein, 9 aa, Homo sapiens
LFPWDIWNL

SEQ ID NO: 220 - protein, 9 aa, Homo sapiens
FPWDIWNLL

SEQ ID NO: 221 - protein, 9 aa, Homo sapiens
DIWNLLESY

SEQ ID NO: 222 - protein, 9 aa, Homo sapiens
ESYSDIKQM

SEQ ID NO: 223 - protein, 9 aa, Homo sapiens
LDTVTFYKL

SEQ ID NO: 224 - protein, 9 aa, Homo sapiens
DTVTFYKLY

SEQ ID NO: 225 - protein, 9 aa, Homo sapiens
YTPSGHSQK

SEQ ID NO: 226 - protein, 9 aa, Homo sapiens
GHSQKQVRL

SEQ ID NO: 227 - protein, 9 aa, Homo sapiens
RLGEVRHPV

SEQ ID NO: 228 - protein, 9 aa, Homo sapiens
GEVRHPVPL

SEQ ID NO: 229 - protein, 9 aa, Homo sapiens
EVRHPVPLR

SEQ ID NO: 230 - protein, 9 aa, Homo sapiens
PWWGAIAAF

SEQ ID NO: 231 - protein, 9 aa, Homo sapiens
FQVQSLPAA

SEQ ID NO: 232 - protein, 9 aa, Homo sapiens
GVAEYSFSY

SEQ ID NO: 233 - protein, 9 aa, Homo sapiens
VAEYSFSYF

SEQ ID NO: 234 - protein, 9 aa, Homo sapiens
AEYSFSYFK

SEQ ID NO: 235 - protein, 9 aa, Homo sapiens
YSFSYFKSL

SEQ ID NO: 236 - protein, 9 aa, Homo sapiens
SFSYFKSLR

SEQ ID NO: 237 - protein, 9 aa, Homo sapiens
SYFKSLRGK

SEQ ID NO: 238 - protein, 9 aa, Homo sapiens
RGKDSGSSF

SEQ ID NO: 239 - protein, 9 aa, Homo sapiens
GEEIVTYDL

SEQ ID NO: 240 - protein, 9 aa, Homo sapiens
GEEIVTYDL

SEQ ID NO: 241 - protein, 9 aa, Homo sapiens
EIVTYDLSL

SEQ ID NO: 242 - protein, 9 aa, Homo sapiens
EIVTYDLSL

SEQ ID NO: 243 - protein, 9 aa, Homo sapiens
VTYDLSLCF

SEQ ID NO: 244 - protein, 9 aa, Homo sapiens
VTYDLSLCF

SEQ ID NO: 245 - protein, 9 aa, Homo sapiens
REMSGGRKP

SEQ ID NO: 246 - protein, 9 aa, Homo sapiens
RWEKAFDCL

SEQ ID NO: 247 - protein, 9 aa, Homo sapiens
WEKAFDCLF

SEQ ID NO: 248 - protein, 9 aa, Homo sapiens
KPLIVFSAV

SEQ ID NO: 249 - protein, 9 aa, Homo sapiens
VFSAVGDTV

SEQ ID NO: 250 - protein, 9 aa, Homo sapiens
AEDEVVCEI

SEQ ID NO: 251 - protein, 9 aa, Homo sapiens
ETDKTSVQV

SEQ ID NO: 252 - protein, 9 aa, Homo sapiens
QVPSPANGV

SEQ ID NO: 253 - protein, 9 aa, Homo sapiens
VPSPANGVI

SEQ ID NO: 254 - protein, 9 aa, Homo sapiens
ETLAAPAGL

SEQ ID NO: 255 - protein, 9 aa, Homo sapiens
DEEEDAPLF

SEQ ID NO: 256 - protein, 9 aa, Homo sapiens
EEEDAPLFL

SEQ ID NO: 257 - protein, 9 aa, Homo sapiens
EEDAPLFLL

SEQ ID NO: 258 - protein, 9 aa, Homo sapiens
DAPLFLLGK

SEQ ID NO: 259 - protein, 9 aa, Homo sapiens
FLLGKKRSL

SEQ ID NO: 260 - protein, 9 aa, Homo sapiens
RSLPNPNQF

SEQ ID NO: 261 - protein, 9 aa, Homo sapiens
LPNPNQFPY

SEQ ID NO: 262 - protein, 9 aa, Homo sapiens
VPSSVTDVI

SEQ ID NO: 263 - protein, 9 aa, Homo sapiens
SSVTDVISL

SEQ ID NO: 264 - protein, 9 aa, Homo sapiens
VTDVISLSL

SEQ ID NO: 265 - protein, 9 aa, Homo sapiens
QSTSSPSGF

SEQ ID NO: 266 - protein, 9 aa, Homo sapiens
STSSPSGFR

SEQ ID NO: 267 - protein, 9 aa, Homo sapiens
HIPFISKSF

SEQ ID NO: 268 - protein, 9 aa, Homo sapiens
IPFISKSFV

SEQ ID NO: 269 - protein, 9 aa, Homo sapiens
PFISKSFVL

SEQ ID NO: 270 - protein, 9 aa, Homo sapiens
ISKSFVLGV

SEQ ID NO: 271 - protein, 9 aa, Homo sapiens
SFVLGVKHR

SEQ ID NO: 272 - protein, 9 aa, Homo sapiens
QRALRAPRI

SEQ ID NO: 273 - protein, 9 aa, Homo sapiens
ALRAPRICF

SEQ ID NO: 274 - protein, 9 aa, Homo sapiens
LRAPRICFF

SEQ ID NO: 275 - protein, 9 aa, Homo sapiens
APRICFFIA

SEQ ID NO: 276 - protein, 9 aa, Homo sapiens
RICFFIARR

SEQ ID NO: 277 - protein, 9 aa, Homo sapiens
FQLPRAQSF

SEQ ID NO: 278 - protein, 9 aa, Homo sapiens
LPRAQSFST

SEQ ID NO: 279 - protein, 9 aa, Homo sapiens
FSTPPAGAV

SEQ ID NO: 280 - protein, 9 aa, Homo sapiens
STPPAGAVW

SEQ ID NO: 281 - protein, 9 aa, Homo sapiens
VWARTPPCL

SEQ ID NO: 282 - protein, 9 aa, Homo sapiens
WARTPPCLL

SEQ ID NO: 283 - protein, 9 aa, Homo sapiens
HSCWVTGTR

SEQ ID NO: 284 - protein, 9 aa, Homo sapiens
HSDSTVSCM

SEQ ID NO: 285 - protein, 9 aa, Homo sapiens
VSCMPLPLR

SEQ ID NO: 286 - protein, 9 aa, Homo sapiens
MPLPLRAST

SEQ ID NO: 287 - protein, 9 aa, Homo sapiens
RASTRRWSR

SEQ ID NO: 288 - protein, 9 aa, Homo sapiens
TRRWSRACL

SEQ ID NO: 289 - protein, 9 aa, Homo sapiens
ETPAWVLCV

SEQ ID NO: 290 - protein, 9 aa, Homo sapiens
TPAWVLCVW

SEQ ID NO: 291 - protein, 9 aa, Homo sapiens
WPSTGWPSM

SEQ ID NO: 292 - protein, 9 aa, Homo sapiens
TGWPSMTCW

SEQ ID NO: 293 - protein, 9 aa, Homo sapiens
WRGLLITAR

SEQ ID NO: 294 - protein, 9 aa, Homo sapiens
HPSCPWPSM

SEQ ID NO: 295 - protein, 9 aa, Homo sapiens
HPGSPSPAA

SEQ ID NO: 296 - protein, 9 aa, Homo sapiens
EEDESWASL

SEQ ID NO: 297 - protein, 9 aa, Homo sapiens
ESWASLADF

SEQ ID NO: 298 - protein, 9 aa, Homo sapiens
TVMMKHVIF

SEQ ID NO: 299 - protein, 9 aa, Homo sapiens
MMKHVIFCR

SEQ ID NO: 300 - protein, 9 aa, Homo sapiens
HVIFCRLHW

SEQ ID NO: 301 - protein, 9 aa, Homo sapiens
VIFCRLHWR

SEQ ID NO: 302 - protein, 9 aa, Homo sapiens
APMCVERLL

SEQ ID NO: 303 - protein, 9 aa, Homo sapiens
VHLNLAAPL

SEQ ID NO: 304 - protein, 9 aa, Homo sapiens
AAPLPHPQV

SEQ ID NO: 305 - protein, 9 aa, Homo sapiens
APLPHPQVA

SEQ ID NO: 306 - protein, 9 aa, Homo sapiens
LPHPQVATA

SEQ ID NO: 307 - protein, 9 aa, Homo sapiens
MEIIQVPDL

SEQ ID NO: 308 - protein, 9 aa, Homo sapiens
EIIQVPDLF

SEQ ID NO: 309 - protein, 9 aa, Homo sapiens
QVPDLFFSR

SEQ ID NO: 310 - protein, 9 aa, Homo sapiens
VPDLFFSRL

SEQ ID NO: 311 - protein, 9 aa, Homo sapiens
RLYTGGEDR

SEQ ID NO: 312 - protein, 9 aa, Homo sapiens
VQIVISSPL

SEQ ID NO: 313 - protein, 9 aa, Homo sapiens
VISSPLLAV

SEQ ID NO: 314 - protein, 9 aa, Homo sapiens
ISSPLLAVR

SEQ ID NO: 315 - protein, 9 aa, Homo sapiens
SPLLAVRGL

SEQ ID NO: 316 - protein, 9 aa, Homo sapiens
LAVRGLEAW

SEQ ID NO: 317 - protein, 9 aa, Homo sapiens
VLEQRFTKR

SEQ ID NO: 318 - protein, 9 aa, Homo sapiens
QRFTKRCPY

SEQ ID NO: 319 - protein, 9 aa, Homo sapiens
RFTKRCPYR

SEQ ID NO: 320 - protein, 9 aa, Homo sapiens
GSSPQPSRR

SEQ ID NO: 321 - protein, 9 aa, Homo sapiens
DFILKPILF

SEQ ID NO: 322 - protein, 9 aa, Homo sapiens
FILKPILFL

SEQ ID NO: 323 - protein, 9 aa, Homo sapiens
KVLKSGQLR

SEQ ID NO: 324 - protein, 9 aa, Homo sapiens
LIIPTGGHL

SEQ ID NO: 325 - protein, 9 aa, Homo sapiens
FTGPGPVSK

SEQ ID NO: 326 - protein, 9 aa, Homo sapiens
GQYLSSNSL

SEQ ID NO: 327 - protein, 9 aa, Homo sapiens
QYLSSNSLL

SEQ ID NO: 328 - protein, 9 aa, Homo sapiens
YLSSNSLLY

SEQ ID NO: 329 - protein, 9 aa, Homo sapiens
LSSNSLLYL

SEQ ID NO: 330 - protein, 9 aa, Homo sapiens
SLLYLGIIK

SEQ ID NO: 331 - protein, 9 aa, Homo sapiens
LLYLGIIKL

SEQ ID NO: 332 - protein, 9 aa, Homo sapiens
LYLGIIKLL

SEQ ID NO: 333 - protein, 9 aa, Homo sapiens
KLLEDDLLI

SEQ ID NO: 334 - protein, 9 aa, Homo sapiens
LLEDDLLIR

SEQ ID NO: 335 - protein, 9 aa, Homo sapiens
LEDDLLIRW

SEQ ID NO: 336 - protein, 9 aa, Homo sapiens
LLIRWQHFL

SEQ ID NO: 337 - protein, 9 aa, Homo sapiens
RWQHFLHTW

SEQ ID NO: 338 - protein, 9 aa, Homo sapiens
QHFLHTWVL

SEQ ID NO: 339 - protein, 9 aa, Homo sapiens
TWVLQSTNL

SEQ ID NO: 340 - protein, 9 aa, Homo sapiens
WVLQSTNLW

SEQ ID NO: 341 - protein, 9 aa, Homo sapiens
LQSTNLWQI

SEQ ID NO: 342 - protein, 9 aa, Homo sapiens
WQIHTGPAL

SEQ ID NO: 343 - protein, 9 aa, Homo sapiens
IHTGPALTL

SEQ ID NO: 344 - protein, 9 aa, Homo sapiens
ALTLPVQSL

SEQ ID NO: 345 - protein, 9 aa, Homo sapiens
LTLPVQSLR

SEQ ID NO: 346 - protein, 9 aa, Homo sapiens
LPVQSLRNL

SEQ ID NO: 347 - protein, 9 aa, Homo sapiens
TPVQPMLAL

SEQ ID NO: 348 - protein, 9 aa, Homo sapiens
IDRHIISSL

SEQ ID NO: 349 - protein, 9 aa, Homo sapiens
RHIISSLRV

SEQ ID NO: 350 - protein, 9 aa, Homo sapiens
AYGEKGYAL

SEQ ID NO: 351 - protein, 9 aa, Homo sapiens
GEKGYALSL

SEQ ID NO: 352 - protein, 9 aa, Homo sapiens
NTKFRSHEF

SEQ ID NO: 353 - protein, 9 aa, Homo sapiens
REKLPTSGL

SEQ ID NO: 354 - protein, 9 aa, Homo sapiens
KLPTSGLKL

SEQ ID NO: 355 - protein, 9 aa, Homo sapiens
LPTSGLKLM

SEQ ID NO: 356 - protein, 9 aa, Homo sapiens
GLKLMGPEV

SEQ ID NO: 357 - protein, 9 aa, Homo sapiens
KLMGPEVQV

SEQ ID NO: 358 - protein, 9 aa, Homo sapiens
LMGPEVQVM

SEQ ID NO: 359 - protein, 9 aa, Homo sapiens
MEDLDLEAV

SEQ ID NO: 360 - protein, 9 aa, Homo sapiens
LDLEAVVVA

SEQ ID NO: 361 - protein, 9 aa, Homo sapiens
LEAVVVAEA

SEQ ID NO: 362 - protein, 9 aa, Homo sapiens
EAVVVAEAV

SEQ ID NO: 363 - protein, 9 aa, Homo sapiens
VVAEAVAEA

SEQ ID NO: 364 - protein, 9 aa, Homo sapiens
AEAVAEATA

SEQ ID NO: 365 - protein, 9 aa, Homo sapiens
AVAEATAGV

SEQ ID NO: 366 - protein, 9 aa, Homo sapiens
AEATAGVAV

SEQ ID NO: 367 - protein, 9 aa, Homo sapiens
GEAEDHHAI

SEQ ID NO: 368 - protein, 9 aa, Homo sapiens
EAEDHHAIL

SEQ ID NO: 369 - protein, 9 aa, Homo sapiens
AEDHHAILP

SEQ ID NO: 370 - protein, 9 aa, Homo sapiens
DHHAILPVI

SEQ ID NO: 371 - protein, 9 aa, Homo sapiens
HHAILPVIA

SEQ ID NO: 372 - protein, 9 aa, Homo sapiens
AILPVIADL

SEQ ID NO: 373 - protein, 9 aa, Homo sapiens
LPVIADLAL

SEQ ID NO: 374 - protein, 9 aa, Homo sapiens
VIADLALTY

SEQ ID NO: 375 - protein, 9 aa, Homo sapiens
IADLALTYL

SEQ ID NO: 376 - protein, 9 aa, Homo sapiens
ADLALVHKM

SEQ ID NO: 377 - protein, 9 aa, Homo sapiens
DLALTYLKR

SEQ ID NO: 378 - protein, 9 aa, Homo sapiens
LALTYLKRW

SEQ ID NO: 379 - protein, 9 aa, Homo sapiens
LTYLKRWIK

SEQ ID NO: 380 - protein, 9 aa, Homo sapiens
ANFDEENAL

SEQ ID NO: 381 - protein, 9 aa, Homo sapiens
NFDEENALL

SEQ ID NO: 382 - protein, 9 aa, Homo sapiens
NALLSNSQL

SEQ ID NO: 383 - protein, 9 aa, Homo sapiens
ALLSNSQLF

SEQ ID NO: 384 - protein, 9 aa, Homo sapiens
TEAVSSQTS

SEQ ID NO: 385 - protein, 9 aa, Homo sapiens
AVSSQTSSL

SEQ ID NO: 386 - protein, 9 aa, Homo sapiens
SSLPPGNAA

SEQ ID NO: 387 - protein, 9 aa, Homo sapiens
SLPPGNAAL

SEQ ID NO: 388 - protein, 9 aa, Homo sapiens
LPEGARGDI

SEQ ID NO: 389 - protein, 9 aa, Homo sapiens
DIANAEPQF

SEQ ID NO: 390 - protein, 9 aa, Homo sapiens
AEPQFQPAA

SEQ ID NO: 391 - protein, 9 aa, Homo sapiens
FQPAAQPGL

SEQ ID NO: 392 - protein, 9 aa, Homo sapiens
QTGDPVMAK

SEQ ID NO: 393 - protein, 9 aa, Homo sapiens
SLNLLKINL

SEQ ID NO: 394 - protein, 9 aa, Homo sapiens
LKINLCLSF

SEQ ID NO: 395 - protein, 9 aa, Homo sapiens
LSFEQAWRF

SEQ ID NO: 396 - protein, 9 aa, Homo sapiens
SFEQAWRFV

SEQ ID NO: 397 - protein, 9 aa, Homo sapiens
FEQAWRFVV

SEQ ID NO: 398 - protein, 9 aa, Homo sapiens
WRFVVPVSL

SEQ ID NO: 399 - protein, 9 aa, Homo sapiens
VPVSLEPCW

SEQ ID NO: 400 - protein, 9 aa, Homo sapiens
LPRRAGLNT

SEQ ID NO: 401 - protein, 9 aa, Homo sapiens
RAGLNTFAL

SEQ ID NO: 402 - protein, 9 aa, Homo sapiens
GLNTFALGL

SEQ ID NO: 403 - protein, 9 aa, Homo sapiens
NTFALGLQK

SEQ ID NO: 404 - protein, 9 aa, Homo sapiens
TIAFKEPSL

SEQ ID NO: 405 - protein, 9 aa, Homo sapiens
KEPSLSPAL

SEQ ID NO: 406 - protein, 9 aa, Homo sapiens
SLSPALPGA

SEQ ID NO: 407 - protein, 9 aa, Homo sapiens
DRQGGGQVL

SEQ ID NO: 408 - protein, 9 aa, Homo sapiens
TLEPGDQAV

SEQ ID NO: 409 - protein, 9 aa, Homo sapiens
LEPGDQAVL

SEQ ID NO: 410 - protein, 9 aa, Homo sapiens
QAVLPPVPL

SEQ ID NO: 411 - protein, 9 aa, Homo sapiens
NPNTPSRLF

SEQ ID NO: 412 - protein, 9 aa, Homo sapiens
MARKIRMNV

SEQ ID NO: 413 - protein, 9 aa, Homo sapiens
KIRMNVPGQ

SEQ ID NO: 414 - protein, 9 aa, Homo sapiens
IRMNVPGQM

SEQ ID NO: 415 - protein, 9 aa, Homo sapiens
RMNVPGQML

SEQ ID NO: 416 - protein, 9 aa, Homo sapiens
MNVPGQMLM

SEQ ID NO: 417 - protein, 9 aa, Homo sapiens
RLPQGVYPV

SEQ ID NO: 418 - protein, 9 aa, Homo sapiens
LPQGVYPVF

SEQ ID NO: 419 - protein, 9 aa, Homo sapiens
GVYPVFASF

SEQ ID NO: 420 - protein, 9 aa, Homo sapiens
VYPVFASFR

SEQ ID NO: 421 - protein, 9 aa, Homo sapiens
YPVFASFRL

SEQ ID NO: 422 - protein, 9 aa, Homo sapiens
QKFNITHSF

SEQ ID NO: 423 - protein, 9 aa, Homo sapiens
RDKGLVETF

SEQ ID NO: 424 - protein, 9 aa, Homo sapiens
KGLVETFYF

SEQ ID NO: 425 - protein, 9 aa, Homo sapiens
GLVETFYFT

SEQ ID NO: 426 - protein, 9 aa, Homo sapiens
VETFYFTEL

SEQ ID NO: 427 - protein, 9 aa, Homo sapiens
ETFYFTELT

SEQ ID NO: 428 - protein, 9 aa, Homo sapiens
LRRSISFMF

SEQ ID NO: 429 - protein, 9 aa, Homo sapiens
RRSISFMFL

SEQ ID NO: 430 - protein, 9 aa, Homo sapiens
SISFMFLSY

SEQ ID NO: 431 - protein, 9 aa, Homo sapiens
KHKLYVSVL

SEQ ID NO: 432 - protein, 9 aa, Homo sapiens
SFMFLSYVR

SEQ ID NO: 433 - protein, 9 aa, Homo sapiens
FLSYVREEA

SEQ ID NO: 434 - protein, 9 aa, Homo sapiens
KLSPIPNPR

SEQ ID NO: 435 - protein, 9 aa, Homo sapiens
SPIPNPRPV

SEQ ID NO: 436 - protein, 9 aa, Homo sapiens
IPNPRPVVF

SEQ ID NO: 437 - protein, 9 aa, Homo sapiens
NPRPVVFGR

SEQ ID NO: 438 - protein, 9 aa, Homo sapiens
VVFGRNHQF

SEQ ID NO: 439 - protein, 9 aa, Homo sapiens
NHQFMQQAV

SEQ ID NO: 440 - protein, 9 aa, Homo sapiens
HQFMQQAVW

SEQ ID NO: 441 - protein, 9 aa, Homo sapiens
QFMQQAVWR

SEQ ID NO: 442 - protein, 9 aa, Homo sapiens
GKYFQVSAL

SEQ ID NO: 443 - protein, 9 aa, Homo sapiens
GKYFQVSAL

SEQ ID NO: 444 - protein, 9 aa, Homo sapiens
KYFQVSALL

SEQ ID NO: 445 - protein, 9 aa, Homo sapiens
KYFQVSALL

SEQ ID NO: 446 - protein, 9 aa, Homo sapiens
YFQVSALLL

SEQ ID NO: 447 - protein, 9 aa, Homo sapiens
YFQVSALLL

SEQ ID NO: 448 - protein, 9 aa, Homo sapiens
VSALLLQLF

SEQ ID NO: 449 - protein, 9 aa, Homo sapiens
VSALLLQLF

SEQ ID NO: 450 - protein, 9 aa, Homo sapiens
ALLLQLFTL

SEQ ID NO: 451 - protein, 9 aa, Homo sapiens
ALLLQLFTL

SEQ ID NO: 452 - protein, 9 aa, Homo sapiens
FTLPYRPGF

SEQ ID NO: 453 - protein, 9 aa, Homo sapiens
FTLPYRPGF

SEQ ID NO: 454 - protein, 9 aa, Homo sapiens
LPYRPGFEV

SEQ ID NO: 455 - protein, 9 aa, Homo sapiens
YRPGFEVSR

SEQ ID NO: 456 - protein, 9 aa, Homo sapiens
RPGFEVSRL

SEQ ID NO: 457 - protein, 9 aa, Homo sapiens
EEHVNRVSL

SEQ ID NO: 458 - protein, 9 aa, Homo sapiens
EHVNRVSLI

SEQ ID NO: 459 - protein, 9 aa, Homo sapiens
SLIGLAGQW

SEQ ID NO: 460 - protein, 9 aa, Homo sapiens
IGLAGQWPR

SEQ ID NO: 461 - protein, 9 aa, Homo sapiens
AGQWPRTSY

SEQ ID NO: 462 - protein, 9 aa, Homo sapiens
RTSYSTRSS

SEQ ID NO: 463 - protein, 9 aa, Homo sapiens
TSYSTRSSK

SEQ ID NO: 464 - protein, 9 aa, Homo sapiens
TRSSKPCSL

SEQ ID NO: 465 - protein, 9 aa, Homo sapiens
CSLTGLPAW

SEQ ID NO: 466 - protein, 9 aa, Homo sapiens
TGLPAWPTK

SEQ ID NO: 467 - protein, 9 aa, Homo sapiens
LPAWPTKGL

SEQ ID NO: 468 - protein, 9 aa, Homo sapiens
AWPTKGLVM

SEQ ID NO: 469 - protein, 9 aa, Homo sapiens
GECGRLWQV

SEQ ID NO: 470 - protein, 9 aa, Homo sapiens
LYLVGFLHL

SEQ ID NO: 471 - protein, 9 aa, Homo sapiens
YLVGFLHLF

SEQ ID NO: 472 - protein, 9 aa, Homo sapiens
FRICLVSAW

SEQ ID NO: 473 - protein, 9 aa, Homo sapiens
LLSIPELQY

SEQ ID NO: 474 - protein, 9 aa, Homo sapiens
LSIPELQYK

SEQ ID NO: 475 - protein, 9 aa, Homo sapiens
SIPELQYKL

SEQ ID NO: 476 - protein, 9 aa, Homo sapiens
QYKLPSHAV

SEQ ID NO: 477 - protein, 9 aa, Homo sapiens
GQAFDQHFI

SEQ ID NO: 478 - protein, 9 aa, Homo sapiens
QAFDQHFIW

SEQ ID NO: 479 - protein, 9 aa, Homo sapiens
ETFRYMIVF

SEQ ID NO: 480 - protein, 9 aa, Homo sapiens
FRYMIVFNY

SEQ ID NO: 481 - protein, 9 aa, Homo sapiens
RYMIVFNYI

SEQ ID NO: 482 - protein, 9 aa, Homo sapiens
IVFNYISSR

SEQ ID NO: 483 - protein, 9 aa, Homo sapiens
SPFMAYGVS

SEQ ID NO: 484 - protein, 9 aa, Homo sapiens
PFMAYGVSF

SEQ ID NO: 485 - protein, 9 aa, Homo sapiens
FMAYGVSFR

SEQ ID NO: 486 - protein, 9 aa, Homo sapiens
MAYGVSFRF

SEQ ID NO: 487 - protein, 9 aa, Homo sapiens
SFRFSKSTL

SEQ ID NO: 488 - protein, 9 aa, Homo sapiens
LTTEYLGPF

SEQ ID NO: 489 - protein, 9 aa, Homo sapiens
TTEYLGPFF

SEQ ID NO: 490 - protein, 9 aa, Homo sapiens
TEYLGPFFL

SEQ ID NO: 491 - protein, 9 aa, Homo sapiens
EYLGPFFLF

SEQ ID NO: 492 - protein, 9 aa, Homo sapiens
GPFFLFPST

SEQ ID NO: 493 - protein, 9 aa, Homo sapiens
PFFLFPSTR

SEQ ID NO: 494 - protein, 9 aa, Homo sapiens
LFSNSQLSL

SEQ ID NO: 495 - protein, 9 aa, Homo sapiens
ERVIPGAAV

SEQ ID NO: 496 - protein, 9 aa, Homo sapiens
IPGAAVCFF

SEQ ID NO: 497 - protein, 9 aa, Homo sapiens
AAVCFFSRK

SEQ ID NO: 498 - protein, 9 aa, Homo sapiens
LRNMRQLKF

SEQ ID NO: 499 - protein, 9 aa, Homo sapiens
NMRQLKFSL

SEQ ID NO: 500 - protein, 9 aa, Homo sapiens
RQLKFSLPL

SEQ ID NO: 501 - protein, 9 aa, Homo sapiens
QLKFSLPLR

SEQ ID NO: 502 - protein, 9 aa, Homo sapiens
LKFSLPLRL

SEQ ID NO: 503 - protein, 9 aa, Homo sapiens
KFSLPLRLL

SEQ ID NO: 504 - protein, 9 aa, Homo sapiens
FSLPLRLLM

SEQ ID NO: 505 - protein, 9 aa, Homo sapiens
SRNECSPFF

SEQ ID NO: 506 - protein, 9 aa, Homo sapiens
ECSPFFPRR

SEQ ID NO: 507 - protein, 9 aa, Homo sapiens
FFPRRPQQL

SEQ ID NO: 508 - protein, 9 aa, Homo sapiens
QHPPAPLEA

SEQ ID NO: 509 - protein, 9 aa, Homo sapiens
HPPAPLEAA

SEQ ID NO: 510 - protein, 9 aa, Homo sapiens
QGMGYIPLV

SEQ ID NO: 511 - protein, 9 aa, Homo sapiens
GMGYIPLVL

SEQ ID NO: 512 - protein, 9 aa, Homo sapiens
GYIPLVLCY

SEQ ID NO: 513 - protein, 9 aa, Homo sapiens
SARPITNPL

SEQ ID NO: 514 - protein, 9 aa, Homo sapiens
RPITNPLNL

SEQ ID NO: 515 - protein, 9 aa, Homo sapiens
HFYQQGTCL

SEQ ID NO: 516 - protein, 9 aa, Homo sapiens
FYQQGTCLL

SEQ ID NO: 517 - protein, 9 aa, Homo sapiens
YQQGTCLLF

SEQ ID NO: 518 - protein, 9 aa, Homo sapiens
MRRDQSLKF

SEQ ID NO: 519 - protein, 9 aa, Homo sapiens
RRDQSLKFF

SEQ ID NO: 520 - protein, 9 aa, Homo sapiens
DQSLKFFSI

SEQ ID NO: 521 - protein, 9 aa, Homo sapiens
SLKFFSIDF

SEQ ID NO: 522 - protein, 9 aa, Homo sapiens
SIHHGVVEL

SEQ ID NO: 523 - protein, 9 aa, Homo sapiens
LNHSPHVQF

SEQ ID NO: 524 - protein, 9 aa, Homo sapiens
NHSPHVQFF

SEQ ID NO: 525 - protein, 9 aa, Homo sapiens
HSPHVQFFF

SEQ ID NO: 526 - protein, 9 aa, Homo sapiens
SPHVQFFFL

SEQ ID NO: 527 - protein, 9 aa, Homo sapiens
HVQFFFLGK

SEQ ID NO: 528 - protein, 9 aa, Homo sapiens
FFFLGKSSV

SEQ ID NO: 529 - protein, 9 aa, Homo sapiens
TSVTTSPQF

SEQ ID NO: 530 - protein, 9 aa, Homo sapiens
VTTSPQFLF

SEQ ID NO: 531 - protein, 9 aa, Homo sapiens
TTSPQFLFY

SEQ ID NO: 532 - protein, 9 aa, Homo sapiens
MVLDFSRMK

SEQ ID NO: 533 - protein, 9 aa, Homo sapiens
VLDFSRMKR

SEQ ID NO: 534 - protein, 9 aa, Homo sapiens
LDFSRMKRL

SEQ ID NO: 535 - protein, 9 aa, Homo sapiens
SRMKRLGTA

SEQ ID NO: 536 - protein, 9 aa, Homo sapiens
RMKRLGTAL

SEQ ID NO: 537 - protein, 9 aa, Homo sapiens
KRLGTALSA

SEQ ID NO: 538 - protein, 9 aa, Homo sapiens
TALSASRPI

SEQ ID NO: 539 - protein, 9 aa, Homo sapiens
ALSASRPIR

SEQ ID NO: 540 - protein, 9 aa, Homo sapiens
SASRPIRSF

SEQ ID NO: 541 - protein, 9 aa, Homo sapiens
ASRPIRSFP

SEQ ID NO: 542 - protein, 9 aa, Homo sapiens
SLSNLHSHL

SEQ ID NO: 543 - protein, 9 aa, Homo sapiens
LHSHLAHSW

SEQ ID NO: 544 - protein, 9 aa, Homo sapiens
SWIDQPGRW

SEQ ID NO: 545 - protein, 9 aa, Homo sapiens
IDQPGRWSW

SEQ ID NO: 546 - protein, 9 aa, Homo sapiens
QPGRWSWAW

SEQ ID NO: 547 - protein, 9 aa, Homo sapiens
GRWSWAWNT

SEQ ID NO: 548 - protein, 9 aa, Homo sapiens
WAWNTDPRL

SEQ ID NO: 549 - protein, 9 aa, Homo sapiens
VSSDSFWEL

SEQ ID NO: 550 - protein, 9 aa, Homo sapiens
SSDSFWELV

SEQ ID NO: 551 - protein, 9 aa, Homo sapiens
SPRSGTTSG

SEQ ID NO: 552 - protein, 9 aa, Homo sapiens
RPVAQSVGT

SEQ ID NO: 553 - protein, 9 aa, Homo sapiens
VAQSVGTVF

SEQ ID NO: 554 - protein, 9 aa, Homo sapiens
GTVFPQPRL

SEQ ID NO: 555 - protein, 9 aa, Homo sapiens
FPQPRLPGV

SEQ ID NO: 556 - protein, 9 aa, Homo sapiens
RLPGVLPRA

SEQ ID NO: 557 - protein, 9 aa, Homo sapiens
GVLPRAPGV

SEQ ID NO: 558 - protein, 9 aa, Homo sapiens
LPRAPGVRL

SEQ ID NO: 559 - protein, 9 aa, Homo sapiens
VRLPHGGHL

SEQ ID NO: 560 - protein, 9 aa, Homo sapiens
FVVGAMTVA

SEQ ID NO: 561 - protein, 9 aa, Homo sapiens
VVGAMTVAF

SEQ ID NO: 562 - protein, 9 aa, Homo sapiens
GAMTVAFFF

SEQ ID NO: 563 - protein, 9 aa, Homo sapiens
MTVAFFFFR

SEQ ID NO: 564 - protein, 9 aa, Homo sapiens
TVAFFFFRY

SEQ ID NO: 565 - protein, 9 aa, Homo sapiens
AFFFFRYGL

SEQ ID NO: 566 - protein, 9 aa, Homo sapiens
QEDPHRMVF

SEQ ID NO: 567 - protein, 9 aa, Homo sapiens
MVFTGSQSW

SEQ ID NO: 568 - protein, 9 aa, Homo sapiens
FTGSQSWPR

SEQ ID NO: 569 - protein, 9 aa, Homo sapiens
AQREQAISY

SEQ ID NO: 570 - protein, 9 aa, Homo sapiens
QREQAISYF

SEQ ID NO: 571 - protein, 9 aa, Homo sapiens
REQAISYFY

SEQ ID NO: 572 - protein, 9 aa, Homo sapiens
ISYFYPTGL

SEQ ID NO: 573 - protein, 9 aa, Homo sapiens
SYFYPTGLL

SEQ ID NO: 574 - protein, 9 aa, Homo sapiens
YFYPTGLLR

SEQ ID NO: 575 - protein, 9 aa, Homo sapiens
KRLSKHLKR

SEQ ID NO: 576 - protein, 9 aa, Homo sapiens
KRLQWLLLR

SEQ ID NO: 577 - protein, 9 aa, Homo sapiens
WLLLRHLQR

SEQ ID NO: 578 - protein, 9 aa, Homo sapiens
LRHLQRQHL

SEQ ID NO: 579 - protein, 9 aa, Homo sapiens
HLQRQHLSK

SEQ ID NO: 580 - protein, 9 aa, Homo sapiens
QRQHLSKRL

SEQ ID NO: 581 - protein, 9 aa, Homo sapiens
HLEAQGTRF

SEQ ID NO: 582 - protein, 9 aa, Homo sapiens
LEAQGTRFF

SEQ ID NO: 583 - protein, 9 aa, Homo sapiens
TRFFLSLSF

SEQ ID NO: 584 - protein, 9 aa, Homo sapiens
RFFLSLSFK

SEQ ID NO: 585 - protein, 9 aa, Homo sapiens
SLSFKVCQR

SEQ ID NO: 586 - protein, 9 aa, Homo sapiens
VHGVSGPVF

SEQ ID NO: 587 - protein, 9 aa, Homo sapiens
GPVFCSLQW

SEQ ID NO: 588 - protein, 9 aa, Homo sapiens
LQWLQPVTW

SEQ ID NO: 589 - protein, 9 aa, Homo sapiens
QWLQPVTWR

SEQ ID NO: 590 - protein, 9 aa, Homo sapiens
WLQPVTWRV

SEQ ID NO: 591 - protein, 9 aa, Homo sapiens
WRVQPCMSW

SEQ ID NO: 592 - protein, 9 aa, Homo sapiens
AMWRNCFFY

SEQ ID NO: 593 - protein, 9 aa, Homo sapiens
MWRNCFFYR

SEQ ID NO: 594 - protein, 9 aa, Homo sapiens
LVARRHYSF

SEQ ID NO: 595 - protein, 9 aa, Homo sapiens
VARRHYSFW

SEQ ID NO: 596 - protein, 9 aa, Homo sapiens
RRHYSFWPI

SEQ ID NO: 597 - protein, 9 aa, Homo sapiens
RHYSFWPIF

SEQ ID NO: 598 - protein, 9 aa, Homo sapiens
FMALDDPVI

SEQ ID NO: 599 - protein, 9 aa, Homo sapiens
MALDDPVIF

SEQ ID NO: 600 - protein, 9 aa, Homo sapiens
ALDDPVIFL

SEQ ID NO: 601 - protein, 9 aa, Homo sapiens
LDDPVIFLL

SEQ ID NO: 602 - protein, 9 aa, Homo sapiens
LCNQKLQAL

SEQ ID NO: 603 - protein, 9 aa, Homo sapiens
NQKLQALAF

SEQ ID NO: 604 - protein, 9 aa, Homo sapiens
IELVQQDNL

SEQ ID NO: 605 - protein, 9 aa, Homo sapiens
LVQQDNLSY

SEQ ID NO: 606 - protein, 9 aa, Homo sapiens
VQQDNLSYR

SEQ ID NO: 607 - protein, 9 aa, Homo sapiens
QQDNLSYRL

SEQ ID NO: 608 - protein, 9 aa, Homo sapiens
QDNLSYRLL

SEQ ID NO: 609 - protein, 9 aa, Homo sapiens
LSYRLLKSF

SEQ ID NO: 610 - protein, 9 aa, Homo sapiens
YRLLKSFSK

SEQ ID NO: 611 - protein, 9 aa, Homo sapiens
RLLKSFSKW

SEQ ID NO: 612 - protein, 9 aa, Homo sapiens
KSFSKWSLV

SEQ ID NO: 613 - protein, 9 aa, Homo sapiens
FSKWSLVQL

SEQ ID NO: 614 - protein, 9 aa, Homo sapiens
LVQLHGIIR

SEQ ID NO: 615 - protein, 9 aa, Homo sapiens
VQLHGIIRK

SEQ ID NO: 616 - protein, 9 aa, Homo sapiens
MSANEDQEF

SEQ ID NO: 617 - protein, 9 aa, Homo sapiens
SANEDQEFF

SEQ ID NO: 618 - protein, 9 aa, Homo sapiens
QEFFISDGT

SEQ ID NO: 619 - protein, 9 aa, Homo sapiens
EFFISDGTR

SEQ ID NO: 620 - protein, 9 aa, Homo sapiens
FISDGTRSI

SEQ ID NO: 621 - protein, 9 aa, Homo sapiens
SDGTRSITL

SEQ ID NO: 622 - protein, 9 aa, Homo sapiens
GTRSITLYL

SEQ ID NO: 623 - protein, 9 aa, Homo sapiens
KEIEAKIKF

SEQ ID NO: 624 - protein, 9 aa, Homo sapiens
IEAKIKFSV

SEQ ID NO: 625 - protein, 9 aa, Homo sapiens
EAKIKFSVL

SEQ ID NO: 626 - protein, 9 aa, Homo sapiens
KIKFSVLPI

SEQ ID NO: 627 - protein, 9 aa, Homo sapiens
FSVLPIPGA

SEQ ID NO: 628 - protein, 9 aa, Homo sapiens
LPIPGADKY

SEQ ID NO: 629 - protein, 9 aa, Homo sapiens
GLKLGSGPY

SEQ ID NO: 630 - protein, 9 aa, Homo sapiens
LKLGSGPYF

SEQ ID NO: 631 - protein, 9 aa, Homo sapiens
KLGSGPYFF

SEQ ID NO: 632 - protein, 9 aa, Homo sapiens
YFFFSNQSL

SEQ ID NO: 633 - protein, 9 aa, Homo sapiens
FFFSNQSLR

SEQ ID NO: 634 - protein, 9 aa, Homo sapiens
FFSNQSLRW

SEQ ID NO: 635 - protein, 9 aa, Homo sapiens
SLRWEISSF

SEQ ID NO: 636 - protein, 9 aa, Homo sapiens
WEISSFNTT

SEQ ID NO: 637 - protein, 9 aa, Homo sapiens
EISSFNTTF

SEQ ID NO: 638 - protein, 9 aa, Homo sapiens
ISSFNTTFW

SEQ ID NO: 639 - protein, 9 aa, Homo sapiens
NTTFWQNVR

SEQ ID NO: 640 - protein, 9 aa, Homo sapiens
RENPVVPIV

SEQ ID NO: 641 - protein, 9 aa, Homo sapiens
NPVVPIVAL

SEQ ID NO: 642 - protein, 9 aa, Homo sapiens
IVALSPPQV

SEQ ID NO: 643 - protein, 9 aa, Homo sapiens
ALSPPQVAW

SEQ ID NO: 644 - protein, 9 aa, Homo sapiens
WPRRPPSPT

SEQ ID NO: 645 - protein, 9 aa, Homo sapiens
RPPSPTAST

SEQ ID NO: 646 - protein, 9 aa, Homo sapiens
SPTASTPST

SEQ ID NO: 647 - protein, 9 aa, Homo sapiens
YVYIPSKTH

SEQ ID NO: 648 - protein, 9 aa, Homo sapiens
VYIPSKTHF

SEQ ID NO: 649 - protein, 9 aa, Homo sapiens
YIPSKTHFF

SEQ ID NO: 650 - protein, 9 aa, Homo sapiens
KTHFFPLCR

SEQ ID NO: 651 - protein, 9 aa, Homo sapiens
HFFPLCRTW

SEQ ID NO: 652 - protein, 9 aa, Homo sapiens
AEEAKNILV

SEQ ID NO: 653 - protein, 9 aa, Homo sapiens
EEAKNILVM

SEQ ID NO: 654 - protein, 9 aa, Homo sapiens
EAKNILVMF

SEQ ID NO: 655 - protein, 9 aa, Homo sapiens
AKNILVMFF

SEQ ID NO: 656 - protein, 9 aa, Homo sapiens
LVMFFLHSR

SEQ ID NO: 657 - protein, 9 aa, Homo sapiens
HSRSGNSEV

SEQ ID NO: 658 - protein, 9 aa, Homo sapiens
SRSGNSEVL

SEQ ID NO: 659 - protein, 9 aa, Homo sapiens
RECFTMDVV

SEQ ID NO: 660 - protein, 9 aa, Homo sapiens
TMDVVIPGL

SEQ ID NO: 661 - protein, 9 aa, Homo sapiens
MDVVIPGLL

SEQ ID NO: 662 - protein, 9 aa, Homo sapiens
DEIITTTVH

SEQ ID NO: 663 - protein, 9 aa, Homo sapiens
EDWGLQTFL

SEQ ID NO: 664 - protein, 9 aa, Homo sapiens
QTFLADSTL

SEQ ID NO: 665 - protein, 9 aa, Homo sapiens
TFLADSTLY

SEQ ID NO: 666 - protein, 9 aa, Homo sapiens
LEQYASKTF

SEQ ID NO: 667 - protein, 9 aa, Homo sapiens
ASKTFPPGS

SEQ ID NO: 668 - protein, 9 aa, Homo sapiens
GVAMEVTKF

SEQ ID NO: 669 - protein, 9 aa, Homo sapiens
MEVTKFCFF

SEQ ID NO: 670 - protein, 9 aa, Homo sapiens
FFLFLDTIR

SEQ ID NO: 671 - protein, 9 aa, Homo sapiens
FLFLDTIRT

SEQ ID NO: 672 - protein, 9 aa, Homo sapiens
LFLDTIRTV

SEQ ID NO: 673 - protein, 9 aa, Homo sapiens
FLDTIRTVT

SEQ ID NO: 674 - protein, 9 aa, Homo sapiens
TRTHQGANL

SEQ ID NO: 675 - protein, 9 aa, Homo sapiens
YTKSRGTQF

SEQ ID NO: 676 - protein, 9 aa, Homo sapiens
KSRGTQFLI

SEQ ID NO: 677 - protein, 9 aa, Homo sapiens
SRGTQFLIL

SEQ ID NO: 678 - protein, 9 aa, Homo sapiens
TQFLILLSW

SEQ ID NO: 679 - protein, 9 aa, Homo sapiens
LLSWQGLAV

SEQ ID NO: 680 - protein, 9 aa, Homo sapiens
SWQGLAVNR

SEQ ID NO: 681 - protein, 9 aa, Homo sapiens
GLPVQSPQV

SEQ ID NO: 682 - protein, 9 aa, Homo sapiens
LPVQSPQVL

SEQ ID NO: 683 - protein, 9 aa, Homo sapiens
VQSPQVLLL

SEQ ID NO: 684 - protein, 9 aa, Homo sapiens
GEGGKRESL

SEQ ID NO: 685 - protein, 9 aa, Homo sapiens
LFVLEKDFF

SEQ ID NO: 686 - protein, 9 aa, Homo sapiens
FVLEKDFFY

SEQ ID NO: 687 - protein, 9 aa, Homo sapiens
LEKDFFYSS

SEQ ID NO: 688 - protein, 9 aa, Homo sapiens
KDFFYSSIY

SEQ ID NO: 689 - protein, 9 aa, Homo sapiens
FFYSSIYQL

SEQ ID NO: 690 - protein, 9 aa, Homo sapiens
SSIYQLDMW

SEQ ID NO: 691 - protein, 9 aa, Homo sapiens
ISLLVYKLF

SEQ ID NO: 692 - protein, 9 aa, Homo sapiens
GGGPGGLFY

SEQ ID NO: 693 - protein, 9 aa, Homo sapiens
GLFYNRWQF

SEQ ID NO: 694 - protein, 9 aa, Homo sapiens
LFYNRWQFW

SEQ ID NO: 695 - protein, 9 aa, Homo sapiens
FYNRWQFWR

SEQ ID NO: 696 - protein, 9 aa, Homo sapiens
VREFLSRKL

SEQ ID NO: 697 - protein, 9 aa, Homo sapiens
REFLSRKLS

SEQ ID NO: 698 - protein, 9 aa, Homo sapiens
FLSRKLSSL

SEQ ID NO: 699 - protein, 9 aa, Homo sapiens
LSRKLSSLR

SEQ ID NO: 700 - protein, 9 aa, Homo sapiens
TEICLPLGF

SEQ ID NO: 701 - protein, 9 aa, Homo sapiens
LPLGFHCKA

SEQ ID NO: 702 - protein, 9 aa, Homo sapiens
HMVHQDSQW

SEQ ID NO: 703 - protein, 9 aa, Homo sapiens
SSTVVSLLF

SEQ ID NO: 704 - protein, 9 aa, Homo sapiens
STVVSLLFF

SEQ ID NO: 705 - protein, 9 aa, Homo sapiens
SLLFFSLFR

SEQ ID NO: 706 - protein, 9 aa, Homo sapiens
HLMSPFLSF

SEQ ID NO: 707 - protein, 9 aa, Homo sapiens
SPFLSFPGC

SEQ ID NO: 708 - protein, 9 aa, Homo sapiens
TGKQQVTVL

SEQ ID NO: 709 - protein, 9 aa, Homo sapiens
GKQQVTVLL

SEQ ID NO: 710 - protein, 9 aa, Homo sapiens
KQQVTVLLR

SEQ ID NO: 711 - protein, 9 aa, Homo sapiens
SLMTKELSV

SEQ ID NO: 712 - protein, 9 aa, Homo sapiens
KELSVLDLF

SEQ ID NO: 713 - protein, 9 aa, Homo sapiens
ELSVLDLFR

SEQ ID NO: 714 - protein, 9 aa, Homo sapiens
SVLDLFRSL

SEQ ID NO: 715 - protein, 9 aa, Homo sapiens
VLDLFRSLL

SEQ ID NO: 716 - protein, 9 aa, Homo sapiens
RSLLAVHQL

SEQ ID NO: 717 - protein, 9 aa, Homo sapiens
QLDQPHGGL

SEQ ID NO: 718 - protein, 9 aa, Homo sapiens
RPSRCCSAL

SEQ ID NO: 719 - protein, 9 aa, Homo sapiens
FEFMDDAKI

SEQ ID NO: 720 - protein, 9 aa, Homo sapiens
FMDDAKILF

SEQ ID NO: 721 - protein, 9 aa, Homo sapiens
GAIGPYSFL

SEQ ID NO: 722 - protein, 9 aa, Homo sapiens
AIGPYSFLF

SEQ ID NO: 723 - protein, 9 aa, Homo sapiens
GPYSFLFIA

SEQ ID NO: 724 - protein, 9 aa, Homo sapiens
YSFLFIAVK

SEQ ID NO: 725 - protein, 9 aa, Homo sapiens
SFLFIAVKL

SEQ ID NO: 726 - protein, 9 aa, Homo sapiens
FLFIAVKLY

SEQ ID NO: 727 - protein, 9 aa, Homo sapiens
CRLSFAQFF

SEQ ID NO: 728 - protein, 9 aa, Homo sapiens
RLSFAQFFV

SEQ ID NO: 729 - protein, 9 aa, Homo sapiens
LSFAQFFVR

SEQ ID NO: 730 - protein, 9 aa, Homo sapiens
FVRHKCQTL

SEQ ID NO: 731 - protein, 9 aa, Homo sapiens
TLRPSSSTL

SEQ ID NO: 732 - protein, 9 aa, Homo sapiens
RPSSSTLRA

SEQ ID NO: 733 - protein, 9 aa, Homo sapiens
STLRASILR

SEQ ID NO: 734 - protein, 9 aa, Homo sapiens
TLRASILRL

SEQ ID NO: 735 - protein, 9 aa, Homo sapiens
RASILRLHF

SEQ ID NO: 736 - protein, 9 aa, Homo sapiens
ASILRLHFL

SEQ ID NO: 737 - protein, 9 aa, Homo sapiens
AALENLNKV

SEQ ID NO: 738 - protein, 9 aa, Homo sapiens
ALENLNKVF

SEQ ID NO: 739 - protein, 9 aa, Homo sapiens
LENLNKVFL

SEQ ID NO: 740 - protein, 9 aa, Homo sapiens
NKVFLPLGL

SEQ ID NO: 741 - protein, 9 aa, Homo sapiens
TALTHNQDF

SEQ ID NO: 742 - protein, 9 aa, Homo sapiens
LTHNQDFSI

SEQ ID NO: 743 - protein, 9 aa, Homo sapiens
THNQDFSIY

SEQ ID NO: 744 - protein, 9 aa, Homo sapiens
HNQDFSIYR

SEQ ID NO: 745 - protein, 9 aa, Homo sapiens
NQDFSIYRL

SEQ ID NO: 746 - protein, 9 aa, Homo sapiens
ASQARSPAF

SEQ ID NO: 747 - protein, 9 aa, Homo sapiens
QARSPAFPK

SEQ ID NO: 748 - protein, 9 aa, Homo sapiens
AFPKPVRPL

SEQ ID NO: 749 - protein, 9 aa, Homo sapiens
FPKPVRPLP

SEQ ID NO: 750 - protein, 9 aa, Homo sapiens
LPAPITRIT

SEQ ID NO: 751 - protein, 9 aa, Homo sapiens
GQSDSSQPL

SEQ ID NO: 752 - protein, 9 aa, Homo sapiens
QSDSSQPLL

SEQ ID NO: 753 - protein, 9 aa, Homo sapiens
LTTGRPQGW

SEQ ID NO: 754 - protein, 9 aa, Homo sapiens
RPQGWQDQA

SEQ ID NO: 755 - protein, 9 aa, Homo sapiens
ITIPIHSAA

SEQ ID NO: 756 - protein, 9 aa, Homo sapiens
TIPIHSAAL

SEQ ID NO: 757 - protein, 9 aa, Homo sapiens
HSGDPGPAW

SEQ ID NO: 758 - protein, 9 aa, Homo sapiens
CPPLPLTTL

SEQ ID NO: 759 - protein, 9 aa, Homo sapiens
PPLPLTTLI

SEQ ID NO: 760 - protein, 9 aa, Homo sapiens
YGDSTARSW

SEQ ID NO: 761 - protein, 9 aa, Homo sapiens
STARSWPSR

SEQ ID NO: 762 - protein, 9 aa, Homo sapiens
GLGDGYRLA

SEQ ID NO: 763 - protein, 9 aa, Homo sapiens
HPSLLPYVF

SEQ ID NO: 764 - protein, 9 aa, Homo sapiens
SLLPYVFVI

SEQ ID NO: 765 - protein, 9 aa, Homo sapiens
LPYVFVIVF

SEQ ID NO: 766 - protein, 9 aa, Homo sapiens
IVFTADQCF

SEQ ID NO: 767 - protein, 9 aa, Homo sapiens
LQWAQLSAL

SEQ ID NO: 768 - protein, 9 aa, Homo sapiens
KVSEARKTL

SEQ ID NO: 769 - protein, 9 aa, Homo sapiens
SEARKTLSF

SEQ ID NO: 770 - protein, 9 aa, Homo sapiens
KTLSFENRK

SEQ ID NO: 771 - protein, 9 aa, Homo sapiens
FENRKEPEA

SEQ ID NO: 772 - protein, 9 aa, Homo sapiens
QPSHVAGPL

SEQ ID NO: 773 - protein, 9 aa, Homo sapiens
SHVAGPLSL

SEQ ID NO: 774 - protein, 9 aa, Homo sapiens
HVAGPLSLQ

SEQ ID NO: 775 - protein, 9 aa, Homo sapiens
SLQEGCTVV

SEQ ID NO: 776 - protein, 9 aa, Homo sapiens
WAPGPDYQR

SEQ ID NO: 777 - protein, 9 aa, Homo sapiens
GPDYQRHSL

SEQ ID NO: 778 - protein, 9 aa, Homo sapiens
DYQRHSLGR

SEQ ID NO: 779 - protein, 9 aa, Homo sapiens
HSLGRSHQW

SEQ ID NO: 780 - protein, 9 aa, Homo sapiens
SGNSPSRTR

SEQ ID NO: 781 - protein, 9 aa, Homo sapiens
RTRRTSPHQ

SEQ ID NO: 782 - protein, 9 aa, Homo sapiens
RRTSPHQGR

SEQ ID NO: 783 - protein, 9 aa, Homo sapiens
SPHQGRSSF

SEQ ID NO: 784 - protein, 9 aa, Homo sapiens
QGRSSFSQR

SEQ ID NO: 785 - protein, 9 aa, Homo sapiens
SSFSQRCPR

SEQ ID NO: 786 - protein, 9 aa, Homo sapiens
SFSQRCPRR

SEQ ID NO: 787 - protein, 9 aa, Homo sapiens
FSQRCPRRW

SEQ ID NO: 788 - protein, 9 aa, Homo sapiens
RRWSEPESA

SEQ ID NO: 789 - protein, 9 aa, Homo sapiens
SAEPGRRGW

SEQ ID NO: 790 - protein, 9 aa, Homo sapiens
GRRGWPPVL

SEQ ID NO: 791 - protein, 9 aa, Homo sapiens
PVLPNSSSW

SEQ ID NO: 792 - protein, 9 aa, Homo sapiens
LPNSSSWTR

SEQ ID NO: 793 - protein, 9 aa, Homo sapiens
SSWTRSVSR

SEQ ID NO: 794 - protein, 9 aa, Homo sapiens
QVRPGSRQR

SEQ ID NO: 795 - protein, 9 aa, Homo sapiens
RRKCPGLQV

SEQ ID NO: 796 - protein, 9 aa, Homo sapiens
RKCPGLQVL

SEQ ID NO: 797 - protein, 9 aa, Homo sapiens
QVLRRHLPR

SEQ ID NO: 798 - protein, 9 aa, Homo sapiens
VLRRHLPRK

SEQ ID NO: 799 - protein, 9 aa, Homo sapiens
RHLPRKTAL

SEQ ID NO: 800 - protein, 9 aa, Homo sapiens
HLPRKTALL

SEQ ID NO: 801 - protein, 9 aa, Homo sapiens
LPRKTALLS

SEQ ID NO: 802 - protein, 9 aa, Homo sapiens
KTALLSTKA

SEQ ID NO: 803 - protein, 9 aa, Homo sapiens
STKAPQNSL

SEQ ID NO: 804 - protein, 9 aa, Homo sapiens
KAPQNSLPK

SEQ ID NO: 805 - protein, 9 aa, Homo sapiens
QQWHRATAV

SEQ ID NO: 806 - protein, 9 aa, Homo sapiens
TAVRKRPER

SEQ ID NO: 807 - protein, 9 aa, Homo sapiens
AVRKRPERL

SEQ ID NO: 808 - protein, 9 aa, Homo sapiens
KRPERLGPL

SEQ ID NO: 809 - protein, 9 aa, Homo sapiens
GPLHRSSSI

SEQ ID NO: 810 - protein, 9 aa, Homo sapiens
RSSSIRSPF

SEQ ID NO: 811 - protein, 9 aa, Homo sapiens
SSSIRSPFL

SEQ ID NO: 812 - protein, 9 aa, Homo sapiens
SPFLPDSSA

SEQ ID NO: 813 - protein, 9 aa, Homo sapiens
SASSSNNSR

SEQ ID NO: 814 - protein, 9 aa, Homo sapiens
TPRRPTPSA

SEQ ID NO: 815 - protein, 9 aa, Homo sapiens
RRPTPSAPF

SEQ ID NO: 816 - protein, 9 aa, Homo sapiens
RPTPSAPFT

SEQ ID NO: 817 - protein, 9 aa, Homo sapiens
TPSAPFTHT

SEQ ID NO: 818 - protein, 9 aa, Homo sapiens
APFTHTTPR

SEQ ID NO: 819 - protein, 9 aa, Homo sapiens
FTHTTPRCW

SEQ ID NO: 820 - protein, 9 aa, Homo sapiens
ALDRQTECF

SEQ ID NO: 821 - protein, 9 aa, Homo sapiens
RQTECFSSY

SEQ ID NO: 822 - protein, 9 aa, Homo sapiens
TECFSSYSA

SEQ ID NO: 823 - protein, 9 aa, Homo sapiens
KTVTAVPSV

SEQ ID NO: 824 - protein, 9 aa, Homo sapiens
TVTAVPSVL

SEQ ID NO: 825 - protein, 9 aa, Homo sapiens
VPSVLHSGL

SEQ ID NO: 826 - protein, 9 aa, Homo sapiens
ALDNVDAPL

SEQ ID NO: 827 - protein, 9 aa, Homo sapiens
DNVDAPLAL

SEQ ID NO: 828 - protein, 9 aa, Homo sapiens
SPQACTWTA

SEQ ID NO: 829 - protein, 9 aa, Homo sapiens
VSTTGSHCW

SEQ ID NO: 830 - protein, 9 aa, Homo sapiens
QAHWAPKAM

SEQ ID NO: 831 - protein, 9 aa, Homo sapiens
HWAPKAMCR

SEQ ID NO: 832 - protein, 9 aa, Homo sapiens
WAPKAMCRW

SEQ ID NO: 833 - protein, 9 aa, Homo sapiens
YTAQQFRNV

SEQ ID NO: 834 - protein, 9 aa, Homo sapiens
AQQFRNVSL

SEQ ID NO: 835 - protein, 9 aa, Homo sapiens
QQFRNVSLL

SEQ ID NO: 836 - protein, 9 aa, Homo sapiens
FRNVSLLQR

SEQ ID NO: 837 - protein, 9 aa, Homo sapiens
SLLQRPHSW

SEQ ID NO: 838 - protein, 9 aa, Homo sapiens
LLQRPHSWR

SEQ ID NO: 839 - protein, 9 aa, Homo sapiens
RPHSWRTPA

SEQ ID NO: 840 - protein, 9 aa, Homo sapiens
WRTPASCPL

SEQ ID NO: 841 - protein, 9 aa, Homo sapiens
RTPASCPLR

SEQ ID NO: 842 - protein, 9 aa, Homo sapiens
TPASCPLRM

SEQ ID NO: 843 - protein, 9 aa, Homo sapiens
APRPQATPA

SEQ ID NO: 844 - protein, 9 aa, Homo sapiens
FFRFDEAPR

SEQ ID NO: 845 - protein, 9 aa, Homo sapiens
FRFDEAPRR

SEQ ID NO: 846 - protein, 9 aa, Homo sapiens
RAAQPRGAK

SEQ ID NO: 847 - protein, 9 aa, Homo sapiens
QPRGAKTKA

SEQ ID NO: 848 - protein, 9 aa, Homo sapiens
KTKATGAQA

SEQ ID NO: 849 - protein, 40 aa, Homo sapiens
NRTTHLCSLILLGMIAQRSFIRGPTQCPLTWCPTRMAWMW

SEQ ID NO: 850 - protein, 23 aa, Homo sapiens
QSLENLINFLPIGEGRSMNLHGI

SEQ ID NO: 851 - protein, 18 aa, Homo sapiens
RVLGSCPPRRFLPPLSAR

SEQ ID NO: 852 - protein, 17 aa, Homo sapiens
AVANRMDQFTFQTDKQR

SEQ ID NO: 853 - protein, 20 aa, Homo sapiens
VMAYGSLDFYIFLDRCLYCY

SEQ ID NO: 854 - protein, 14 aa, Homo sapiens
VIFAALPILFQVPT

SEQ ID NO: 855 - protein, 11 aa, Homo sapiens
HRLPFWLKSVL

SEQ ID NO: 856 - protein, 14 aa, Homo sapiens
SRKNCGTWLMVLER

SEQ ID NO: 857 - protein, 9 aa, Homo sapiens
KKYKVEYVL

SEQ ID NO: 858 - protein, 35 aa, Homo sapiens
DSILSCTARTLKMGTSSTAFSEPTRTRWKCILPSY

SEQ ID NO: 859 - protein, 13 aa, Homo sapiens
AYKDFLWCFPFSL

SEQ ID NO: 860 - protein, 22 aa, Homo sapiens
LELFLSRALRALHVLWNGFQLH

SEQ ID NO: 861 - protein, 13 aa, Homo sapiens
ERARDIQPLCPCR

SEQ ID NO: 862 - protein, 17 aa, Homo sapiens
IDQKFVQQVSFKAPGAF

SEQ ID NO: 863 - protein, 9 aa, Homo sapiens
DMESSPTHR

SEQ ID NO: 864 - protein, 28 aa, Homo sapiens
ELRSKSGHRLFPISPETLHFPVSRHLGS

SEQ ID NO: 865 - protein, 16 aa, Homo sapiens
ARHTERTALLPGLPAA

SEQ ID NO: 866 - protein, 17 aa, Homo sapiens
ASPRRPAGWRCENGRLL

SEQ ID NO: 867 - protein, 14 aa, Homo sapiens
MKKLPITYLSLLSI

SEQ ID NO: 868 - protein, 13 aa, Homo sapiens
KQGGLPLPLLCGA

SEQ ID NO: 869 - protein, 15 aa, Homo sapiens
RAPGVPDSLPGAGAR

SEQ ID NO: 870 - protein, 9 aa, Homo sapiens
RVCCPTSPK

SEQ ID NO: 871 - protein, 18 aa, Homo sapiens
VLFLTLNEVFLILQVHGT

SEQ ID NO: 872 - protein, 9 aa, Homo sapiens
YPDDGMGSS

SEQ ID NO: 873 - protein, 18 aa, Homo sapiens
RSSLTAHSMREIHGIAGT

SEQ ID NO: 874 - protein, 9 aa, Homo sapiens
RALINSVQL

SEQ ID NO: 875 - protein, 11 aa, Homo sapiens
DRLAQSVFLSR

SEQ ID NO: 876 - protein, 9 aa, Homo sapiens
QTCSQPAAR

SEQ ID NO: 877 - protein, 13 aa, Homo sapiens
HKILTANSLSSVF

SEQ ID NO: 878 - protein, 21 aa, Homo sapiens
SHGHGRIALYSCRQAPEAPRR

SEQ ID NO: 879 - protein, 12 aa, Homo sapiens
QEILERQPTLFF

SEQ ID NO: 880 - protein, 15 aa, Homo sapiens
YPMDSPEPGGAGSTL

SEQ ID NO: 881 - protein, 13 aa, Homo sapiens
LEFEDFDVFIFFR

SEQ ID NO: 882 - protein, 17 aa, Homo sapiens
AAAMTRTQLLSTGLLQW

SEQ ID NO: 883 - protein, 19 aa, Homo sapiens
DTTAIDVQYLFLPPGGAPV

SEQ ID NO: 884 - protein, 25 aa, Homo sapiens
EQRLQQEDFLRVQQLSAAGPLRPQL

SEQ ID NO: 885 - protein, 14 aa, Homo sapiens
DGHQPLEQYPEAIL

SEQ ID NO: 886 - protein, 15 aa, Homo sapiens
KSSPSMAPLPTPRSC

SEQ ID NO: 887 - protein, 13 aa, Homo sapiens
TSRPLSLSSFWAR

SEQ ID NO: 888 - protein, 18 aa, Homo sapiens
SDGSLSDLWAPAGPSIWW

SEQ ID NO: 889 - protein, 9 aa, Homo sapiens
MEAMLLYLV

SEQ ID NO: 890 - protein, 17 aa, Homo sapiens
KENNKLEDLRFSSEENM

SEQ ID NO: 891 - protein, 9 aa, Homo sapiens
SAHCPGSLR

SEQ ID NO: 892 - protein, 14 aa, Homo sapiens
TACVPHRTRGSNSI

SEQ ID NO: 893 - protein, 10 aa, Homo sapiens
EERFALVLVL

SEQ ID NO: 894 - protein, 11 aa, Homo sapiens
ASRQGPAPAAV

SEQ ID NO: 895 - protein, 23 aa, Homo sapiens
NVLNKSNEVFPLILASQMYVPGK

SEQ ID NO: 896 - protein, 23 aa, Homo sapiens
VSTPSLSSLTFLPLYVIPARSRA

SEQ ID NO: 897 - protein, 35 aa, Homo sapiens
IPLPLIKSSLSPLSELSVPAAPGPSFLPFSSGPPP

SEQ ID NO: 898 - protein, 29 aa, Homo sapiens
REHCFSDFLAGSLRKQMKVLRITKMPCFR

SEQ ID NO: 899 - protein, 14 aa, Homo sapiens
KFDFSYPGIATSSL

SEQ ID NO: 900 - protein, 28 aa, Homo sapiens
FGKPYFIKFIFKSANGICCRRKKALHCL

SEQ ID NO: 901 - protein, 9 aa, Homo sapiens
STPTPSLHL

SEQ ID NO: 902 - protein, 9 aa, Homo sapiens
KELQGQCFL

SEQ ID NO: 903 - protein, 36 aa, Homo sapiens
HRMWRLWTHEVEHYCILLFPWDIWNLLESYSDIKQM

SEQ ID NO: 904 - protein, 32 aa, Homo sapiens
LDTVTFYKLYTPSGHSQKQVRLGEVRHPVPLR

SEQ ID NO: 905 - protein, 9 aa, Homo sapiens
PWWGAIAAF

SEQ ID NO: 906 - protein, 9 aa, Homo sapiens
FQVQSLPAA

SEQ ID NO: 907 - protein, 22 aa, Homo sapiens
GVAEYSFSYFKSLRGKDSGSSF

SEQ ID NO: 908 - protein, 13 aa, Homo sapiens
GEEIVTYDLSLCF

SEQ ID NO: 909 - protein, 20 aa, Homo sapiens
REMSGGRKPLIVFSAVGDTV

SEQ ID NO: 910 - protein, 9 aa, Homo sapiens
AEDEVVCEI

SEQ ID NO: 911 - protein, 17 aa, Homo sapiens
ETDKTSVQVPSPANGVI

SEQ ID NO: 912 - protein, 10 aa, Homo sapiens
RWEKAFDCLF

SEQ ID NO: 913 - protein, 9 aa, Homo sapiens
ETLAAPAGL

SEQ ID NO: 914 - protein, 25 aa, Homo sapiens
DEEEDAPLFLLGKKRSLPNPNQFPY

SEQ ID NO: 915 - protein, 13 aa, Homo sapiens
VPSSVTDVISLSL

SEQ ID NO: 916 - protein, 10 aa, Homo sapiens
QSTSSPSGFR

SEQ ID NO: 917 - protein, 16 aa, Homo sapiens
HIPFISKSFVLGVKHR

SEQ ID NO: 918 - protein, 16 aa, Homo sapiens
QRALRAPRICFFIARR

SEQ ID NO: 919 - protein, 26 aa, Homo sapiens
FQLPRAQSFSTPPAGAVWARTPPCLL

SEQ ID NO: 920 - protein, 9 aa, Homo sapiens
HSCWVTGTR

SEQ ID NO: 921 - protein, 25 aa, Homo sapiens
HSDSTVSCMPLPLRASTRRWSRACL

SEQ ID NO: 922 - protein, 29 aa, Homo sapiens
ETPAWVLCVWPSTGWPSMTCWRGLLITAR

SEQ ID NO: 923 - protein, 9 aa, Homo sapiens
HPSCPWPSM

SEQ ID NO: 924 - protein, 9 aa, Homo sapiens
HPGSPSPAA

SEQ ID NO: 925 - protein, 12 aa, Homo sapiens
EEDESWASLADF

SEQ ID NO: 926 - protein, 15 aa, Homo sapiens
TVMMKHVIFCRLHWR

SEQ ID NO: 927 - protein, 9 aa, Homo sapiens
APMCVERLL

SEQ ID NO: 928 - protein, 17 aa, Homo sapiens
VHLNLAAPLPHPQVATA

SEQ ID NO: 929 - protein, 21 aa, Homo sapiens
MEIIQVPDLFFSRLYTGGEDR

SEQ ID NO: 930 - protein, 18 aa, Homo sapiens
VQIVISSPLLAVRGLEAW

SEQ ID NO: 931 - protein, 13 aa, Homo sapiens
VLEQRFTKRCPYR

SEQ ID NO: 932 - protein, 9 aa, Homo sapiens
GSSPQPSRR

SEQ ID NO: 933 - protein, 10 aa, Homo sapiens
DFILKPILFL

SEQ ID NO: 934 - protein, 9 aa, Homo sapiens
KVLKSGQLR

SEQ ID NO: 935 - protein, 9 aa, Homo sapiens
LIIPTGGHL

SEQ ID NO: 936 - protein, 9 aa, Homo sapiens
FTGPGPVSK

SEQ ID NO: 937 - protein, 59 aa, Homo sapiens
GQYLSSNSLLYLGIIKLLEDDLLIRWQHFLHTWVLQSTNLWQIHTGPALTLPVQSLRNL

SEQ ID NO: 938 - protein, 9 aa, Homo sapiens
TPVQPMLAL

SEQ ID NO: 939 - protein, 11 aa, Homo sapiens
IDRHIISSLRV

SEQ ID NO: 940 - protein, 11 aa, Homo sapiens
AYGEKGYALSL

SEQ ID NO: 941 - protein, 22 aa, Homo sapiens
GEAEDHHAILPVIADLALVHKM

SEQ ID NO: 942 - protein, 26 aa, Homo sapiens
GEAEDHHAILPVIADLALTYLKRWIK

SEQ ID NO: 943 - protein, 9 aa, Homo sapiens
NTKFRSHEF

SEQ ID NO: 944 - protein, 42 aa, Homo sapiens
REKLPTSGLKLMGPEVQVMEDLDLEAVVVAEAVAEATAGVAV

SEQ ID NO: 945 - protein, 16 aa, Homo sapiens
ANFDEENALLSNSQLF

SEQ ID NO: 946 - protein, 18 aa, Homo sapiens
TEAVSSQTSSLPPGNAAL

SEQ ID NO: 947 - protein, 24 aa, Homo sapiens
LPEGARGDIANAEPQFQPAAQPGL

SEQ ID NO: 948 - protein, 9 aa, Homo sapiens
QTGDPVMAK

SEQ ID NO: 949 - protein, 29 aa, Homo sapiens
SLNLLKINLCLSFEQAWRFVVPVSLEPCW

SEQ ID NO: 950 - protein, 16 aa, Homo sapiens
LPRRAGLNTFALGLQK

SEQ ID NO: 951 - protein, 16 aa, Homo sapiens
TIAFKEPSLSPALPGA

SEQ ID NO: 952 - protein, 9 aa, Homo sapiens
DRQGGGQVL

SEQ ID NO: 953 - protein, 15 aa, Homo sapiens
TLEPGDQAVLPPVPL

SEQ ID NO: 954 - protein, 9 aa, Homo sapiens
NPNTPSRLF

SEQ ID NO: 955 - protein, 15 aa, Homo sapiens
MARKIRMNVPGQMLM

SEQ ID NO: 956 - protein, 15 aa, Homo sapiens
RLPQGVYPVFASFRL

SEQ ID NO: 957 - protein, 9 aa, Homo sapiens
QKFNITHSF

SEQ ID NO: 958 - protein, 15 aa, Homo sapiens
RDKGLVETFYFTELT

SEQ ID NO: 959 - protein, 17 aa, Homo sapiens
LRRSISFMFLSYVREEA

SEQ ID NO: 960 - protein, 9 aa, Homo sapiens
KHKLYVSVL

SEQ ID NO: 961 - protein, 26 aa, Homo sapiens
KLSPIPNPRPVVFGRNHQFMQQAVWR

SEQ ID NO: 962 - protein, 22 aa, Homo sapiens
GKYFQVSALLLQLFTLPYRPGF

SEQ ID NO: 963 - protein, 27 aa, Homo sapiens
GKYFQVSALLLQLFTLPYRPGFEVSRL

SEQ ID NO: 964 - protein, 44 aa, Homo sapiens
EEHVNRVSLIGLAGQWPRTSYSTRSSKPCSLTGLPAWPTKGLVM

SEQ ID NO: 965 - protein, 9 aa, Homo sapiens
GECGRLWQV

SEQ ID NO: 966 - protein, 10 aa, Homo sapiens
LYLVGFLHLF

SEQ ID NO: 967 - protein, 9 aa, Homo sapiens
FRICLVSAW

SEQ ID NO: 968 - protein, 16 aa, Homo sapiens
LLSIPELQYKLPSHAV

SEQ ID NO: 969 - protein, 10 aa, Homo sapiens
GQAFDQHFIW

SEQ ID NO: 970 - protein, 15 aa, Homo sapiens
ETFRYMIVFNYISSR

SEQ ID NO: 971 - protein, 17 aa, Homo sapiens
SPFMAYGVSFRFSKSTL

SEQ ID NO: 972 - protein, 16 aa, Homo sapiens
LTTEYLGPFFLFPSTR

SEQ ID NO: 973 - protein, 9 aa, Homo sapiens
LFSNSQLSL

SEQ ID NO: 974 - protein, 15 aa, Homo sapiens
ERVIPGAAVCFFSRK

SEQ ID NO: 975 - protein, 17 aa, Homo sapiens
LRNMRQLKFSLPLRLLM

SEQ ID NO: 976 - protein, 16 aa, Homo sapiens
SRNECSPFFPRRPQQL

SEQ ID NO: 977 - protein, 10 aa, Homo sapiens
QHPPAPLEAA

SEQ ID NO: 978 - protein, 12 aa, Homo sapiens
QGMGYIPLVLCY

SEQ ID NO: 979 - protein, 11 aa, Homo sapiens
SARPITNPLNL

SEQ ID NO: 980 - protein, 11 aa, Homo sapiens
HFYQQGTCLLF

SEQ ID NO: 981 - protein, 14 aa, Homo sapiens
MRRDQSLKFFSIDF

SEQ ID NO: 982 - protein, 9 aa, Homo sapiens
SIHHGVVEL

SEQ ID NO: 983 - protein, 17 aa, Homo sapiens
LNHSPHVQFFFLGKSSV

SEQ ID NO: 984 - protein, 12 aa, Homo sapiens
TSVTTSPQFLFY

SEQ ID NO: 985 - protein, 25 aa, Homo sapiens
MVLDFSRMKRLGTALSASRPIRSFP

SEQ ID NO: 986 - protein, 30 aa, Homo sapiens
SLSNLHSHLAHSWIDQPGRWSWAWNTDPRL

SEQ ID NO: 987 - protein, 10 aa, Homo sapiens
VSSDSFWELV

SEQ ID NO: 988 - protein, 9 aa, Homo sapiens
SPRSGTTSG

SEQ ID NO: 989 - protein, 34 aa, Homo sapiens
RPVAQSVGTVFPQPRLPGVLPRAPGVRLPHGGHL

SEQ ID NO: 990 - protein, 17 aa, Homo sapiens
FVVGAMTVAFFFFRYGL

SEQ ID NO: 991 - protein, 17 aa, Homo sapiens
QEDPHRMVFTGSQSWPR

SEQ ID NO: 992 - protein, 27 aa, Homo sapiens
KRLSKHLKRLQWLLLRHLQRQHLSKRL

SEQ ID NO: 993 - protein, 16 aa, Homo sapiens
KRLSKHLKRLQWLLLR

SEQ ID NO: 994 - protein, 17 aa, Homo sapiens
AQREQAISYFYPTGLLR

SEQ ID NO: 995 - protein, 20 aa, Homo sapiens
HLEAQGTRFFLSLSFKVCQR

SEQ ID NO: 996 - protein, 28 aa, Homo sapiens
VHGVSGPVFCSLQWLQPVTWRVQPCMSW

SEQ ID NO: 997 - protein, 10 aa, Homo sapiens
AMWRNCFFYR

SEQ ID NO: 998 - protein, 13 aa, Homo sapiens
LVARRHYSFWPIF

SEQ ID NO: 999 - protein, 22 aa, Homo sapiens
FMALDDPVIFLLCNQKLQALAF

SEQ ID NO: 1000 - protein, 31 aa, Homo sapiens
IELVQQDNLSYRLLKSFSKWSLVQLHGIIRK

SEQ ID NO: 1001 - protein, 22 aa, Homo sapiens
MSANEDQEFFISDGTRSITLYL

SEQ ID NO: 1002 - protein, 20 aa, Homo sapiens
KEIEAKIKFSVLPIPGADKY

SEQ ID NO: 1003 - protein, 33 aa, Homo sapiens
GLKLGSGPYFFFSNQSLRWEISSFNTTFWQNVR

SEQ ID NO: 1004 - protein, 32 aa, Homo sapiens
RENPVVPIVALSPPQVAWPRRPPSPTASTPST

SEQ ID NO: 1005 - protein, 17 aa, Homo sapiens
YVYIPSKTHFFPLCRTW

SEQ ID NO: 1006 - protein, 23 aa, Homo sapiens
AEEAKNILVMFFLHSRSGNSEVL

SEQ ID NO: 1007 - protein, 14 aa, Homo sapiens
RECFTMDVVIPGLL

SEQ ID NO: 1008 - protein, 9 aa, Homo sapiens
DEIITTTVH

SEQ ID NO: 1009 - protein, 15 aa, Homo sapiens
EDWGLQTFLADSTLY

SEQ ID NO: 1010 - protein, 13 aa, Homo sapiens
LEQYASKTFPPGS

SEQ ID NO: 1011 - protein, 30 aa, Homo sapiens
GVAMEVTKFCFFLFLDTIRTVTRTHQGANL

SEQ ID NO: 1012 - protein, 22 aa, Homo sapiens
YTKSRGTQFLILLSWQGLAVNR

SEQ ID NO: 1013 - protein, 12 aa, Homo sapiens
GLPVQSPQVLLL

SEQ ID NO: 1014 - protein, 9 aa, Homo sapiens
GEGGKRESL

SEQ ID NO: 1015 - protein, 19 aa, Homo sapiens
LFVLEKDFFYSSIYQLDMW

SEQ ID NO: 1016 - protein, 9 aa, Homo sapiens
ISLLVYKLF

SEQ ID NO: 1017 - protein, 16 aa, Homo sapiens
GGGPGGLFYNRWQFWR

SEQ ID NO: 1018 - protein, 13 aa, Homo sapiens
VREFLSRKLSSLR

SEQ ID NO: 1019 - protein, 13 aa, Homo sapiens
TEICLPLGFHCKA

SEQ ID NO: 1020 - protein, 9 aa, Homo sapiens
HMVHQDSQW

SEQ ID NO: 1021 - protein, 14 aa, Homo sapiens
SSTVVSLLFFSLFR

SEQ ID NO: 1022 - protein, 12 aa, Homo sapiens
HLMSPFLSFPGC

SEQ ID NO: 1023 - protein, 11 aa, Homo sapiens
TGKQQVTVLLR

SEQ ID NO: 1024 - protein, 29 aa, Homo sapiens
SLMTKELSVLDLFRSLLAVHQLDQPHGGL

SEQ ID NO: 1025 - protein, 9 aa, Homo sapiens
RPSRCCSAL

SEQ ID NO: 1026 - protein, 11 aa, Homo sapiens
FEFMDDAKILF

SEQ ID NO: 1027 - protein, 16 aa, Homo sapiens
GAIGPYSFLFIAVKLY

SEQ ID NO: 1028 - protein, 34 aa, Homo sapiens
CRLSFAQFFVRHKCQTLRPSSSTLRASILRLHFL

SEQ ID NO: 1029 - protein, 15 aa, Homo sapiens
AALENLNKVFLPLGL

SEQ ID NO: 1030 - protein, 14 aa, Homo sapiens
TALTHNQDFSIYRL

SEQ ID NO: 1031 - protein, 24 aa, Homo sapiens
ASQARSPAFPKPVRPLPAPITRIT

SEQ ID NO: 1032 - protein, 22 aa, Homo sapiens
GQSDSSQPLLTTGRPQGWQDQA

SEQ ID NO: 1033 - protein, 10 aa, Homo sapiens
ITIPIHSAAL

SEQ ID NO: 1034 - protein, 9 aa, Homo sapiens
HSGDPGPAW

SEQ ID NO: 1035 - protein, 10 aa, Homo sapiens
CPPLPLTTLI

SEQ ID NO: 1036 - protein, 12 aa, Homo sapiens
YGDSTARSWPSR

SEQ ID NO: 1037 - protein, 9 aa, Homo sapiens
GLGDGYRLA

SEQ ID NO: 1038 - protein, 19 aa, Homo sapiens
HPSLLPYVFVIVFTADQCF

SEQ ID NO: 1039 - protein, 9 aa, Homo sapiens
LQWAQLSAL

SEQ ID NO: 1040 - protein, 19 aa, Homo sapiens
KVSEARKTLSFENRKEPEA

SEQ ID NO: 1041 - protein, 18 aa, Homo sapiens
QPSHVAGPLSLQEGCTVV

SEQ ID NO: 1042 - protein, 18 aa, Homo sapiens
WAPGPDYQRHSLGRSHQW

SEQ ID NO: 1043 - protein, 34 aa, Homo sapiens
SGNSPSRTRRTSPHQGRSSFSQRCPRRWSEPESA

SEQ ID NO: 1044 - protein, 25 aa, Homo sapiens
SAEPGRRGWPPVLPNSSSWTRSVSR

SEQ ID NO: 1045 - protein, 40 aa, Homo sapiens
QVRPGSRQRRKCPGLQVLRRHLPRKTALLSTKAPQNSLPK

SEQ ID NO: 1046 - protein, 42 aa, Homo sapiens
QQWHRATAVRKRPERLGPLHRSSSIRSPFLPDSSASSSNNSR

SEQ ID NO: 1047 - protein, 19 aa, Homo sapiens
TPRRPTPSAPFTHTTPRCW

SEQ ID NO: 1048 - protein, 14 aa, Homo sapiens
ALDRQTECFSSYSA

SEQ ID NO: 1049 - protein, 14 aa, Homo sapiens
KTVTAVPSVLHSGL

SEQ ID NO: 1050 - protein, 11 aa, Homo sapiens
ALDNVDAPLAL

SEQ ID NO: 1051 - protein, 9 aa, Homo sapiens
SPQACTWTA

SEQ ID NO: 1052 - protein, 9 aa, Homo sapiens
VSTTGSHCW

SEQ ID NO: 1053 - protein, 12 aa, Homo sapiens
QAHWAPKAMCRW

SEQ ID NO: 1054 - protein, 28 aa, Homo sapiens
YTAQQFRNVSLLQRPHSWRTPASCPLRM

SEQ ID NO: 1055 - protein, 9 aa, Homo sapiens
APRPQATPA

SEQ ID NO: 1056 - protein, 12 aa, Homo sapiens
RAAQPRGAKTKA

SEQ ID NO: 1057 - protein, 10 aa, Homo sapiens
FFRFDEAPRR

SEQ ID NO: 1058 - protein, 17 aa, Homo sapiens
RAAQPRGAKTKATGAQA

SEQ ID NO: 1059 - protein, 12 aa, Homo sapiens
CAFDNYGQNFVF

SEQ ID NO: 1060 - protein, 14 aa, Homo sapiens
CAVRSSYNTDKLIF

SEQ ID NO: 1061 - protein, 15 aa, Homo sapiens
CAGGMTTDSWGKLQF

SEQ ID NO: 1062 - protein, 15 aa, Homo sapiens
CAFDRGSSNAGKLIF

SEQ ID NO: 1063 - protein, 12 aa, Homo sapiens
CAGLPYGQNFVF

SEQ ID NO: 1064 - protein, 18 aa, Homo sapiens
CVVNIPLYSGAGSYQLTF

SEQ ID NO: 1065 - protein, 12 aa, Homo sapiens
CAAREYGNKLVF

SEQ ID NO: 1066 - protein, 20 aa, Homo sapiens
CIVGGTALENYGGSQGNLIF

SEQ ID NO: 1067 - protein, 17 aa, Homo sapiens
CAMREGTLRGSQGNLIF

SEQ ID NO: 1068 - protein, 14 aa, Homo sapiens
CAMSLYSSASKIIF

SEQ ID NO: 1069 - protein, 14 aa, Homo sapiens
CAMSGVDSSYKLIF

SEQ ID NO: 1070 - protein, 15 aa, Homo sapiens
CAASRYGNNFNKFYF

SEQ ID NO: 1071 - protein, 12 aa, Homo sapiens
CAASAPGNQFYF

SEQ ID NO: 1072 - protein, 15 aa, Homo sapiens
CAASTSYSGNTPLVF

SEQ ID NO: 1073 - protein, 13 aa, Homo sapiens
CAVRGSNDYKLSF

SEQ ID NO: 1074 - protein, 16 aa, Homo sapiens
CATDKGSGGSNYKLTF

SEQ ID NO: 1075 - protein, 11 aa, Homo sapiens
CAFWSAYKLSF

SEQ ID NO: 1076 - protein, 13 aa, Homo sapiens
CAGRDNYGQNFVF

SEQ ID NO: 1077 - protein, 12 aa, Homo sapiens
CAVRNTGNQFYF

SEQ ID NO: 1078 - protein, 9 aa, Homo sapiens
CATDDDMRF

SEQ ID NO: 1079 - protein, 13 aa, Homo sapiens
CAAPGNYGQNFVF

SEQ ID NO: 1080 - protein, 12 aa, Homo sapiens
CAGIVRDDKIIF

SEQ ID NO: 1081 - protein, 11 aa, Homo sapiens
CAALGGDKIIF

SEQ ID NO: 1082 - protein, 15 aa, Homo sapiens
CAMIEWDTGRRALIF

SEQ ID NO: 1083 - protein, 14 aa, Homo sapiens
CAFFEYDNNARLMF

SEQ ID NO: 1084 - protein, 13 aa, Homo sapiens
CAFLQGGSEKLVF

SEQ ID NO: 1085 - protein, 18 aa, Homo sapiens
CAFMKHEDSGAGSYQLTF

SEQ ID NO: 1086 - protein, 14 aa, Homo sapiens
CALTEVDSSYKLIF

SEQ ID NO: 1087 - protein, 15 aa, Homo sapiens
CAPSLMDTGRRALTF

SEQ ID NO: 1088 - protein, 12 aa, Homo sapiens
CASGLPDTPLVF

SEQ ID NO: 1089 - protein, 15 aa, Homo sapiens
CAMSAPDTGGFKTIF

SEQ ID NO: 1090 - protein, 12 aa, Homo sapiens
CAESEGADGLTF

SEQ ID NO: 1091 - protein, 11 aa, Homo sapiens
CAVRNDYKLSF

SEQ ID NO: 1092 - protein, 14 aa, Homo sapiens
CAVITTDSWGKLQF

SEQ ID NO: 1093 - protein, 10 aa, Homo sapiens
CAVLGNQFYF

SEQ ID NO: 1094 - protein, 13 aa, Homo sapiens
CADPTDSWGKLQF

SEQ ID NO: 1095 - protein, 14 aa, Homo sapiens
CVVIGPFNDYKLSF

SEQ ID NO: 1096 - protein, 17 aa, Homo sapiens
CAFMKHEDTGGNTPLVF

SEQ ID NO: 1097 - protein, 14 aa, Homo sapiens
CAVKGAGANSKLTF

SEQ ID NO: 1098 - protein, 14 aa, Homo sapiens
CAYYPWNTGNQFYF

SEQ ID NO: 1099 - protein, 12 aa, Homo sapiens
CAVSDYGQNFVF

SEQ ID NO: 1100 - protein, 13 aa, Homo sapiens
CVGGGGSNYKLTF

SEQ ID NO: 1101 - protein, 15 aa, Homo sapiens
CAFMKPDSGTYKYIF

SEQ ID NO: 1102 - protein, 10 aa, Homo sapiens
CAVLGNQFYF

SEQ ID NO: 1103 - protein, 15 aa, Homo sapiens
CAYRSPTALNEKLTF

SEQ ID NO: 1104 - protein, 15 aa, Homo sapiens
CAVRAFGYNFNKFYF

SEQ ID NO: 1105 - protein, 12 aa, Homo sapiens
CAVSVRDDKIIF

SEQ ID NO: 1106 - protein, 11 aa, Homo sapiens
CAPQGAQKLVF

SEQ ID NO: 1107 - protein, 12 aa, Homo sapiens
CAYMNNNNDMRF

SEQ ID NO: 1108 - protein, 11 aa, Homo sapiens
CALSEVDRLMF

SEQ ID NO: 1109 - protein, 11 aa, Homo sapiens
CAEMEGTALIF

SEQ ID NO: 1110 - protein, 13 aa, Homo sapiens
CALSDPDMEKLTF

SEQ ID NO: 1111 - protein, 13 aa, Homo sapiens
CVVSFQGTDKLIF

SEQ ID NO: 1112 - protein, 11 aa, Homo sapiens
CAAERDDKIIF

SEQ ID NO: 1113 - protein, 15 aa, Homo sapiens
CAVRDSGAGSYQLTF

SEQ ID NO: 1114 - protein, 10 aa, Homo sapiens
CAYSNYQLIW

SEQ ID NO: 1115 - protein, 13 aa, Homo sapiens
CAVESGNTGKLIF

SEQ ID NO: 1116 - protein, 12 aa, Homo sapiens
CAGYPGYSTLTF

SEQ ID NO: 1117 - protein, 11 aa, Homo sapiens
CAVRINYKLSF

SEQ ID NO: 1118 - protein, 14 aa, Homo sapiens
CAVRDGHRDDKIIF

SEQ ID NO: 1119 - protein, 12 aa, Homo sapiens
CAYNTGGFKTIF

SEQ ID NO: 1120 - protein, 10 aa, Homo sapiens
CAESQGNLIF

SEQ ID NO: 1121 - protein, 14 aa, Homo sapiens
CAYRSPVSGNQFYF

SEQ ID NO: 1122 - protein, 13 aa, Homo sapiens
CAGGSGTASKLTF

SEQ ID NO: 1123 - protein, 14 aa, Homo sapiens
CAGAGEAGAQKLVF

SEQ ID NO: 1124 - protein, 14 aa, Homo sapiens
CATDRDQGAQKLVF

SEQ ID NO: 1125 - protein, 9 aa, Homo sapiens
CTSNYQLIW

SEQ ID NO: 1126 - protein, 11 aa, Homo sapiens
CAVSGGNQFYF

SEQ ID NO: 1127 - protein, 10 aa, Homo sapiens
CAVENYQLIW

SEQ ID NO: 1128 - protein, 14 aa, Homo sapiens
CAMREGGSQGNLIF

SEQ ID NO: 1129 - protein, 11 aa, Homo sapiens
CAMRGYQKVTF

SEQ ID NO: 1130 - protein, 15 aa, Homo sapiens
CAVRDSGAGSYQLTF

SEQ ID NO: 1131 - protein, 15 aa, Homo sapiens
CAEENSGAGSYQLTF

SEQ ID NO: 1132 - protein, 12 aa, Homo sapiens
CAESMSYQKVTF

SEQ ID NO: 1133 - protein, 11 aa, Homo sapiens
CILDNNNDMRF

SEQ ID NO: 1134 - protein, 14 aa, Homo sapiens
CAVNAVDSNYQLIW

SEQ ID NO: 1135 - protein, 15 aa, Homo sapiens
CALSEANEGTYKYIF

SEQ ID NO: 1136 - protein, 12 aa, Homo sapiens
CAASDGGNKLVF

SEQ ID NO: 1137 - protein, 13 aa, Homo sapiens
CAVSPYNNNDMRF

SEQ ID NO: 1138 - protein, 14 aa, Homo sapiens
CAEYAMDSSYKLIF

SEQ ID NO: 1139 - protein, 14 aa, Homo sapiens
CAYRTPLRDDKIIF

SEQ ID NO: 1140 - protein, 17 aa, Homo sapiens
CAADTNYYTGANSKLTF

SEQ ID NO: 1141 - protein, 14 aa, Homo sapiens
CAVKGFFGNEKLTF

SEQ ID NO: 1142 - protein, 12 aa, Homo sapiens
CAVRDNNARLMF

SEQ ID NO: 1143 - protein, 15 aa, Homo sapiens
CAVSDPDTGFQKLVF

SEQ ID NO: 1144 - protein, 12 aa, Homo sapiens
CAVDITWNDMRF

SEQ ID NO: 1145 - protein, 14 aa, Homo sapiens
CAVPLRADLTKLVF

SEQ ID NO: 1146 - protein, 17 aa, Homo sapiens
CALSDRDGGTSYGKLTF

SEQ ID NO: 1147 - protein, 15 aa, Homo sapiens
CALSEATSGTYKYIF

SEQ ID NO: 1148 - protein, 13 aa, Homo sapiens
CAMRAFGNEKLTF

SEQ ID NO: 1149 - protein, 13 aa, Homo sapiens
CASSPMDRDEQYF

SEQ ID NO: 1150 - protein, 16 aa, Homo sapiens
CASSQESVGSNQPQHF

SEQ ID NO: 1151 - protein, 16 aa, Homo sapiens
CASSPGTGVTKDGYTF

SEQ ID NO: 1152 - protein, 11 aa, Homo sapiens
CASEGVHEQFF

SEQ ID NO: 1153 - protein, 14 aa, Homo sapiens
CASSPVGWGNTIYF

SEQ ID NO: 1154 - protein, 16 aa, Homo sapiens
CASSYKAEPIYNEQFF

SEQ ID NO: 1155 - protein, 14 aa, Homo sapiens
CASSQLGETGELFF

SEQ ID NO: 1156 - protein, 12 aa, Homo sapiens
CGGQGYHTEAFF

SEQ ID NO: 1157 - protein, 19 aa, Homo sapiens
CASSLEAPGVISGANVLTF

SEQ ID NO: 1158 - protein, 14 aa, Homo sapiens
CASSLDLRQNEQFF

SEQ ID NO: 1159 - protein, 16 aa, Homo sapiens
CSVDWYGGLTNTEAFF

SEQ ID NO: 1160 - protein, 16 aa, Homo sapiens
CASSAEGSWGQETQYF

SEQ ID NO: 1161 - protein, 15 aa, Homo sapiens
CASSQGGGGNQPQHF

SEQ ID NO: 1162 - protein, 15 aa, Homo sapiens
CASSFLAGGPNEQYF

SEQ ID NO: 1163 - protein, 14 aa, Homo sapiens
CASSTMGDYYEQYF

SEQ ID NO: 1164 - protein, 15 aa, Homo sapiens
CASSPWGAGNQPQHF

SEQ ID NO: 1165 - protein, 13 aa, Homo sapiens
CAINSGFGSPLHF

SEQ ID NO: 1166 - protein, 11 aa, Homo sapiens
CSASQIYEQYF

SEQ ID NO: 1167 - protein, 13 aa, Homo sapiens
CASSFSLQYEQYF

SEQ ID NO: 1168 - protein, 13 aa, Homo sapiens
CASSLWAGNTIYF

SEQ ID NO: 1169 - protein, 16 aa, Homo sapiens
CSVEDLGAGVSNEQFF

SEQ ID NO: 1170 - protein, 12 aa, Homo sapiens
CASSYEDHEQYF

SEQ ID NO: 1171 - protein, 17 aa, Homo sapiens
CASSLFGLAGVEETQYF

SEQ ID NO: 1172 - protein, 20 aa, Homo sapiens
CASSQAISDREVWDQETQYF

SEQ ID NO: 1173 - protein, 14 aa, Homo sapiens
CASSLAGGSYEQYF

SEQ ID NO: 1174 - protein, 15 aa, Homo sapiens
CASSFGGDGYNEQFF

SEQ ID NO: 1175 - protein, 15 aa, Homo sapiens
CASSLRDRGNQPQHF

SEQ ID NO: 1176 - protein, 9 aa, Homo sapiens
CSASEGYTF

SEQ ID NO: 1177 - protein, 15 aa, Homo sapiens
CASSYPTSGYNEQFF

SEQ ID NO: 1178 - protein, 16 aa, Homo sapiens
CASSSGLAGASNEQFF

SEQ ID NO: 1179 - protein, 14 aa, Homo sapiens
CASSLVAGTDGYTF

SEQ ID NO: 1180 - protein, 13 aa, Homo sapiens
CASSLIGLAEQFF

SEQ ID NO: 1181 - protein, 13 aa, Homo sapiens
CSVEKGNNYGYTF

SEQ ID NO: 1182 - protein, 15 aa, Homo sapiens
CASSLSQGIYYEQYF

SEQ ID NO: 1183 - protein, 15 aa, Homo sapiens
CASRSDRVTEHTQYF

SEQ ID NO: 1184 - protein, 12 aa, Homo sapiens
CASSDGSYEQYF

SEQ ID NO: 1185 - protein, 12 aa, Homo sapiens
CASSDSGTEAFF

SEQ ID NO: 1186 - protein, 16 aa, Homo sapiens
CASSLTPPGSYNEQFF

SEQ ID NO: 1187 - protein, 14 aa, Homo sapiens
CASSRDPQPDTQYF

SEQ ID NO: 1188 - protein, 14 aa, Homo sapiens
CASSLVAGTDGYTF

SEQ ID NO: 1189 - protein, 15 aa, Homo sapiens
CASSLWTSGYETQYF

SEQ ID NO: 1190 - protein, 14 aa, Homo sapiens
CASSLASGNYEQYF

SEQ ID NO: 1191 - protein, 14 aa, Homo sapiens
CASSFDRAEYEQYF

SEQ ID NO: 1192 - protein, 16 aa, Homo sapiens
CASSLSAGAFSDTQYF

SEQ ID NO: 1193 - protein, 16 aa, Homo sapiens
CASSLPTQGGLIEQFF

SEQ ID NO: 1194 - protein, 13 aa, Homo sapiens
CASSLTGTDTQYF

SEQ ID NO: 1195 - protein, 12 aa, Homo sapiens
CASSQGAAEAFF

SEQ ID NO: 1196 - protein, 13 aa, Homo sapiens
CASSFGTPYEQYF

SEQ ID NO: 1197 - protein, 16 aa, Homo sapiens
CSATGEAWTGWNEQFF

SEQ ID NO: 1198 - protein, 16 aa, Homo sapiens
CASSMTSGSPYNEQFF

SEQ ID NO: 1199 - protein, 15 aa, Homo sapiens
CATQDSLFMNTEAFF

SEQ ID NO: 1200 - protein, 13 aa, Homo sapiens
CASSFVTSYEQYF

SEQ ID NO: 1201 - protein, 13 aa, Homo sapiens
CASSISSTGELFF

SEQ ID NO: 1202 - protein, 14 aa, Homo sapiens
CASSLGQYNSPLHF

SEQ ID NO: 1203 - protein, 15 aa, Homo sapiens
CASSRVTSGHNEQFF

SEQ ID NO: 1204 - protein, 15 aa, Homo sapiens
CASSLGVRAQKTQYF

SEQ ID NO: 1205 - protein, 15 aa, Homo sapiens
CATGPRGSSYNEQFF

SEQ ID NO: 1206 - protein, 13 aa, Homo sapiens
CASSQSPGGEQFF

SEQ ID NO: 1207 - protein, 12 aa, Homo sapiens
CASTPPRGPQHF

SEQ ID NO: 1208 - protein, 15 aa, Homo sapiens
CASSVAGSYGDTQYF

SEQ ID NO: 1209 - protein, 14 aa, Homo sapiens
CASSPTGLVYEQFF

SEQ ID NO: 1210 - protein, 15 aa, Homo sapiens
CASSQEVGIWQTQYF

SEQ ID NO: 1211 - protein, 13 aa, Homo sapiens
CASSLFRETEAFF

SEQ ID NO: 1212 - protein, 14 aa, Homo sapiens
CASSLTGETEKLFF

SEQ ID NO: 1213 - protein, 14 aa, Homo sapiens
CASSQGQEKETQYF

SEQ ID NO: 1214 - protein, 14 aa, Homo sapiens
CASRFDGSNQPQHF

SEQ ID NO: 1215 - protein, 15 aa, Homo sapiens
CASSHELTRADTQYF

SEQ ID NO: 1216 - protein, 16 aa, Homo sapiens
CASSAPVWEGTGELFF

SEQ ID NO: 1217 - protein, 13 aa, Homo sapiens
CASSLGDSTEAFF

SEQ ID NO: 1218 - protein, 14 aa, Homo sapiens
CASSLEASTDTQYF

SEQ ID NO: 1219 - protein, 16 aa, Homo sapiens
CASSLAPGTTNEKLFF

SEQ ID NO: 1220 - protein, 11 aa, Homo sapiens
CASSGFGKLFF

SEQ ID NO: 1221 - protein, 17 aa, Homo sapiens
CASSNSIGSADTDTQYF

SEQ ID NO: 1222 - protein, 17 aa, Homo sapiens
CASMGGLAGGYADTQYF

SEQ ID NO: 1223 - protein, 14 aa, Homo sapiens
CASRPLAAQETQYF

SEQ ID NO: 1224 - protein, 14 aa, Homo sapiens
CASSEGEGFYGYTF

SEQ ID NO: 1225 - protein, 16 aa, Homo sapiens
CASSDTAGSSYNEQFF

SEQ ID NO: 1226 - protein, 16 aa, Homo sapiens
CASSFGRDLNTGELFF

SEQ ID NO: 1227 - protein, 15 aa, Homo sapiens
CAIDPTGSLNQPQHF

SEQ ID NO: 1228 - protein, 13 aa, Homo sapiens
CASSRQQGVEQYV

SEQ ID NO: 1229 - protein, 10 aa, Homo sapiens
CATQNNEQFF

SEQ ID NO: 1230 - protein, 16 aa, Homo sapiens
CASSLEVGLAPNEQFF

SEQ ID NO: 1231 - protein, 13 aa, Homo sapiens
CASSYSPGYEQYF

SEQ ID NO: 1232 - protein, 14 aa, Homo sapiens
CASSLITGDTEAFF

SEQ ID NO: 1233 - protein, 11 aa, Homo sapiens
CASSPRDEQFF

(19) United States
(12) Patent Application Publication
(10) Pub. No.: US 2023/0212243 A1
(43) Pub. Date: Jul. 6, 2023

(54) Neoantigenic Epitopes Associated with SF3B1 Mutations

(71) Applicants: Institut Curie, Paris (FR); Institut National de la Santé et de la Recherche Médicale, Paris (FR)

(72) Inventors: Olivier Lantz, Paris (FR); Marc-Henri Stern, Paris (FR); Jérémy Bigot, Eaunes (FR); Alexandre Houy, Vitry sur Seine (FR); Ana Ines Lalanne, Paris (FR)

(73) Assignees: Institut Curie, Paris (FR); Institut National de la Santé et de la Recherche Médicale, Paris (FR)

(21) Appl. No.: 17/924,013

(22) PCT Filed: May 12, 2021
(86) PCT No.: PCT/EP2021/062723

§ 371 Date: Nov. 8, 2022

(30) Foreign Application Priority Data
May 12, 2020 (EP) 20305477.0

(51) Int. Cl. C07K 14/47; A61K 39/00

(52) U.S. Cl. CPC C07K 14/4748; A61K 39/0011

(57) ABSTRACT

The present application relates to a tumor specific neoantigenic peptide, wherein said peptide is encoded by a part of an ORF sequence from a transcript associated with a SF3B1 or a SF3B1-like mutation, comprises at least 8 amino acids and binds at least one MHC molecule with an affinity of less than 500 nM; and is not expressed in normal healthy cells. The present application further relates to vaccine or immunogenic composition, antibodies, T cell receptors, polynucleotides, vectors and immune cells derived thereof as well as their use in therapy of cancer.

### FIELD OF THE DISCLOSURE

The present disclosure provides shared neoantigenic peptides resulting from mutations in the splicing factor SF3B1 or resulting from SF3B1-like mutations, nucleic acids, vaccines, antibodies and immune cells that can be used in cancer therapy.

### BACKGROUND

Harnessing the immune system to generate effective responses against tumors is a central goal of cancer immunotherapy.

Part of the effective immune response involves T lymphocytes specific for tumor antigens. T cell activation requires their interaction with antigen-presenting cells (APCs), commonly dendritic cells (DCs), expressing TCR-cognate peptides presented in the context of a major histocompatibility molecule (MHC) and co-stimulation signals. Neoplasms often contain infiltrating T lymphocytes reactive with tumor cells. Subsequently, activated T cells can recognize peptide-MHC complexes presented by all cell types, even malignant cells.

Although not spontaneously effective, once stimulated with various immunomodulators the immune system can destroy large tumors as well as the disseminated disease.

However, the efficiency of immune responses against tumors is severely dampened by various immunosuppressive strategies developed by tumors, e.g., tumor cells express receptors that provide inhibitory signals to infiltrating T cells, or they secrete inhibitory cytokines.

The development of checkpoint blockade therapy has provided means to bypass some of these mechanisms, leading to more efficient killing of cancer cells. The promising results yielded by this approach have opened up new avenues for the development of T cell-based immunotherapy.

Checkpoint inhibitors are, however, effective in a minority of patients and only in limited types of cancer. Indeed, the clinical response to anti-checkpoint treatments is loosely correlated with the number of somatic mutations present in the tumor suggesting that the number of neo-epitopes expressed by the tumor is important to generate an efficient immune response. Unfortunately, most of these neo-epitopes correspond to passenger mutations that are different in each tumor and specific for each patient. In the absence of spontaneous response, inducing an immune response to such epitopes requires personalized vaccines which are costly and logistically complicated to set up. For that reasons, public (shared between individuals) epitopes deriving from germinally encoded antigens aberrantly expressed in tumors with limited expression in normal tissues such as onco-testis antigens are often used in vaccine strategies. However, many of these antigens are also expressed in the thymus potentially leading to deletion of the high avidity T cells. New tumor neoantigens would be of interest and might improve or reduce the cost of cancer therapy, in particular in the case of vaccination and adoptive cell therapy.

Identification of immunogenic neoantigens would be of particular relevance in the case of uveal melanoma (UM), a rare disease (#600 case/year in France) with a dismal prognosis once metastatic, which happens in ˜30% of the cases and for which no therapy is available. Indeed, contrary to skin melanoma, UMs display very few somatic mutations (<20 per exome) and are accordingly resistant to anti-checkpoint immunomodulation.

### SUMMARY

The present disclosure provides a tumor specific neoantigenic peptide, wherein said peptide:
is encoded by a part of an ORF sequence from a transcript associated with SF3B1 mutation or with SF3B1-like mutation present in a SF3B1 or in a SF3B1-like mutant tumor sample;
comprises at least 8 amino acids and binds at least one MHC molecule with an affinity of less than 500 nM; and
is not expressed in normal healthy cells.

In some embodiments, SF3B1-like mutation(s) include mutation(s) of the SUGP1 gene.

More particularly, the present application discloses tumor specific neoantigenic peptide, notably specific for tumor associated with a mutation of SF3B1, selected from SEQ ID NO: 1-1058.

In one embodiment, the tumor neoantigenic peptide is 8 or 9 amino acids long, notably 8 to 11, and binds to at least one MHC class I molecule of said subject.

Typically, the neoantigenic peptides bind MHC class I or class II with a binding affinity Kd of less than about 10⁻⁶, 10⁻⁷, 10⁻⁸ or 10⁻⁹ M (lower numbers indicating higher binding affinity).

Typically, the neoantigenic peptides bind MHC class I with a binding affinity of less than 0.5% percentile rank score predicted by NetMHCpan 4.0.

According to the present disclosure, the SF3B1 mutant tumor can notably be selected from uveal melanoma, hematopathies (in particular hematological malignancies), breast cancers, skin melanoma, renal cell carcinoma, pulmonary adenocarcinoma, hepatocarcinoma, pancreatic carcinoma, endometrial cancers and uveal melanoma, optionally wherein the tumor is SF3B1 mutant associated with hematological malignancies, uveal melanoma and/or pulmonary adenocarcinoma.

In some embodiments said neoantigenic peptides are expressed in at least 30%, 40%, 50%, 60%, 70% or more of subjects from a population of subjects suffering from a SF3B1 mutant tumor and more particularly from a population of subjects suffering from uveal melanoma (UM).

The present disclosure also encompasses:
a population of autologous dendritic cells or antigen presenting cells that have been pulsed with one or more of the neoantigenic peptides as herein defined, or transfected with a polynucleotide encoding one or more of such peptides;
a vaccine or immunogenic composition, notably a sterile vaccine or immunogenic composition, capable of raising a specific T-cell response comprising
   a. one or more neoantigenic peptides as defined in the present disclosure, optionally wherein the neoantigenic peptides are modified, or complexed with HLA complexes;
   b. one or more polynucleotides encoding a neoantigenic peptide as herein defined, optionally wherein the one or more polynucleotides are linked to a heterologous regulatory control nucleotide sequence; or
   c. a population of autologous dendritic cells or antigen presenting cells (notably artificial APC) that have been pulsed or loaded with one or more of the peptides as herein defined,
optionally in combination with a physiologically or pharmacologically acceptable buffer, carrier, excipient, immunostimulant and/or adjuvant.
   an antibody, or an antigen-binding fragment thereof, a T cell receptor (TCR), or a chimeric antigen receptor (CAR) that has been selected for its binding affinity to a neoantigenic peptide as herein defined, or a composition comprising such antibody, antigen-binding fragment thereof, TCR or CAR.
   a polynucleotide encoding a neoantigenic peptide, a vector comprising thereof, an antibody, a CAR or a TCR as herein defined, typically operatively linked to a heterologous regulatory control nucleotide sequence, and a vector encoding such polynucleotide, or a vaccine or immunogenic composition comprising such polynucleotide or vector;
   an immune cell, or a population or immune cells that targets one or more neoantigenic peptides, as herein defined, wherein the population of immune cells preferably targets a plurality of different tumor neoantigenic peptides as herein disclosed, or a composition comprising such immune cells or population of immune cells optionally in combination with a physiologically or pharmacologically acceptable buffer, carrier, excipient, immunostimulant and/or adjuvant.

Typically, the antibody or antigen-binding fragment thereof, TCR or CAR binds a neoantigenic peptide, in association with an MHC molecule (notably a MHC class I molecule), with a Kd affinity of about 10⁻⁶ M or less.

In some embodiments, the T cell receptor can be made soluble and fused to an antibody fragment directed to a T cell antigen, optionally wherein the targeted antigen is CD3 or CD16.

In some embodiments, the antibody can be a multispecific antibody that further targets at least an immune cell antigen, optionally wherein the immune cell is a T cell, a NK cell or a dendritic cell, optionally wherein the targeted antigen is CD3, CD16, CD30 or a TCR. In any of the embodiments relating to an antibody, the antibody can be chimeric, humanized, or human, and may be IgG, e.g. IgG1, IgG2, IgG3, IgG4.

The immune cell can be typically a T cell or a NK cell, a CD4+ and/or CD8+ cell, a TILs/tumor derived CD8 T cells, a central memory CD8+ T cells, a Treg, a MAIT, or a γδ T cell. The cell can also be autologous or allogenic.

The T cell can comprises comprise a recombinant antigen receptor selected from T cell receptor and chimeric antigen receptor as herein defined, wherein the antigen is a tumor neoantigenic receptor as herein disclosed.

The present disclosure also encompasses a method of producing an antibody, TCR or CAR that specifically binds a neoantigenic peptide as herein defined and comprising the step of selecting an antibody, TCR or CAR that binds to a tumor neoantigen peptide of the present disclosure, in association with an MHC or HLA molecule (notably an MHC class I molecule), optionally with a Kd binding affinity of about 10⁻⁶ M or less. Antibodies, TCRs and CARs selected by said method are also part of the present application, and thus any references to antibodies, TCRs or CARs herein also means an antibody, TCR or CAR that has been selected by said method.

A polynucleotide encoding a neoantigenic peptide as herein defined, or encoding an antibody, a CAR or a TCR as herein defined, optionally linked to a heterologous regulatory control sequence are also part of the present application.

As per the present disclosure, the neoantigenic peptide, the population of dendritic cells, the vaccine or immunogenic composition, the polynucleotide or the vector encoding the peptide can be used in cancer vaccination therapy of a subject; or for treating cancer in a subject suffering from cancer or at risk of cancer; or can be used for inhibiting proliferation of cancer cells.

Typically, the peptide(s) bind at least one MHC molecule, notably at least one MHC class I of said subject.

As per the present disclosure, the antibody or the antigen-binding fragment thereof, the multispecific antibody, the TCR, the CAR, the polynucleotide, or the vector encoding such antibody, TCR or CAR, as herein defined can be used in the treatment of cancer in a subject in need thereof, the subject suffering from cancer or at risk of cancer, or can be used for inhibiting proliferation of cancer cells. Still as per the present disclosure, the population of immune cells as herein defined can be used in cell therapy of a subject suffering from cancer or at risk of cancer, or can be used for inhibiting proliferation of cancer cells.

Particularly, the neoantigenic peptide, the population of dendritic cells, the vaccine or immunogenic composition, the polynucleotide or the vector encoding the peptide, the antibody or the antigen-binding fragment thereof, the multispecific antibody, the TCR, the CAR, the polynucleotide, or the vector encoding such antibody, TCR or CAR or the population of immune cells (collectively referenced herein as the “Cancer Therapeutic Products”) are used in the treatment of a subject who is suffering from a SF3B1 mutant tumor. In one embodiment, the subject is suffering from an SF3B1 mutant associated uveal melanoma or is at risk of suffering from an SF3B1 mutant associated uveal melanoma.

Pharmaceutical compositions comprising any of the foregoing, optionally with a sterile pharmaceutically acceptable excipient(s), carrier, and/or buffer are also contemplated as well as methods of using them.

In any of the embodiments described herein, the Cancer Therapeutic Products as above defined can be administered in combination with at least one further therapeutic agent. Such further therapeutic agent can typically be a chemotherapeutic agent, or an immunotherapeutic agent.

For example, according to the present disclosure, any of the Cancer Therapeutic Products can be administered in combination with an anti-immunosuppressive/immunostimulatory agent. For example, the subject is further administered with one or more checkpoint inhibitors typically selected from PD-1 inhibitors, PD-L1 inhibitors, Lag-3 inhibitors, Tim-3 inhibitors, TIGIT inhibitors, BTLA inhibitors, V-domain Ig suppressor of T-cell activation (VISTA) inhibitors and CTLA-4 inhibitors, or IDO inhibitors.

Various embodiments of the methods, neoantigenic peptides and Cancer Therapeutic Products are described in detailed below. Except for alternatives clearly mentioned, combinations of such embodiments are encompassed by the present application.

### DETAILED DISCLOSURE

Splicing factor (SF) mutations represent an important class of driver mutations in human cancers and affect about 50 to 60% of patients with a myeloid neoplasm with myelodysplasia (Yoshida, K. et al. Frequent pathway mutations of splicing machinery in myelodysplasia. Nature 478, 64-69 (2011)).

More particularly, twenty percent of the uveal melanoma (UM) tumors harbor a mutation in the SF3B1 splicing factors generating over 1000 new splice junctions. SF3B1 mutations lead to an upstream shift of the splice acceptor sites leading to inclusion of intronic sequences in the mRNA. The resulting additional amino-acids and the frame-shift, which is often associated potentially generate a large number of public neo-epitopes. The proteins or polypeptides resulting from splicing anomalies may be in low amounts as the abnormal proteins are probably short lived. However, from an immunological perspective, the abnormal polypeptides generated by a premature stop codon are detected during the first-pass translation quality check by the ribosomes. These abnormal polypeptides represent defective ribosomal products (DRiPs) that are preferentially loaded on the MHC-class I molecules. In the absence of premature stop codon, the proteins harboring the amino-acid insertion may be misfolded and are also rapidly degraded and targeted to the MHC class I loading compartment. Both processes efficiently generate potential neo-epitopes

The inventors have shown herein that among metastatic UM patients, only those whose tumor tissues expressed a mutated SF3B1, displayed memory CD8 T cells with specificities for SF3B1-induced neo-epitopes. They also demonstrated that SF3B1mutant UM cell lines were recognized by those specific T cell clones demonstrating that the neo-epitopes are expressed by tumor cells in a way that can be recognized by CD8 T cells. These results provide evidence that SF3B1mutation-induced neo-epitopes are excellent tumor specific therapeutic targets for SF3B1 mutant-associated tumors and notably for uveal melanoma. Indeed, SF3B lmutation-induced neo-epitopes are public (i.e.: shared between individuals) epitopes while deriving from germinally encoded antigens aberrantly expressed in tumors. Said neoantigens are further tumor specific (having thus no or limited expression in normal tissues and in particular in thymus).

The present application therefore proposes neoantigenic epitopes shared among a population of patients suffering from a SF3B1 mutant associated tumor. Considering the poor therapeutic arsenal for treatment for such tumors, notably in the cases of uveal melanoma, and the cost associated with their developments the present application is of great clinical relevance.

### Definitions

According to the present disclosure, the term “normal” refers to the healthy state or the conditions in a healthy subject, tissue, or cell, i.e., non-pathological conditions, wherein “healthy” preferably means non-cancerous.

Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor but some, like leukemia, do not.

Malignant tumor is essentially synonymous with cancer. Malignancy, malignant neoplasm, and malignant tumor are essentially synonymous with cancer.

As used herein, the term “tumor” or “tumor disease” refers to an abnormal growth of cells (called neoplastic cells, tumorigenous cells or tumor cells) preferably forming a swelling or lesion. By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

A benign tumor is a tumor that lacks all three of the malignant properties of a cancer. Thus, by definition, a benign tumor does not grow in an unlimited, aggressive manner, does not invade surrounding tissues, and does not spread to non-adjacent tissues (metastasize).

Neoplasm is an abnormal mass of tissue as a result of neoplasia. Neoplasia (new growth in Greek) is the abnormal proliferation of cells. The growth of the cells exceeds, and is uncoordinated with that of the normal tissues around it. The growth persists in the same excessive manner even after cessation of the stimuli. It usually causes a lump or tumor. Neoplasms may be benign, pre-malignant or malignant.

Splicing factor 3B subunit 1 is a protein (UniProtKB—075533) that in humans is encoded by the SF3B1 gene (Ensembl:ENSG00000115524). This gene encodes subunit 1 of the splicing factor 3b protein complex. Splicing factor 3b, together with splicing factor 3a and a 12S RNA unit, forms the U2 small nuclear ribonucleoproteins complex (U2 snRNP). The splicing factor 3b/3a complex binds pre-mRNA upstream of the intron's branch site in a sequence independent manner and may anchor the U2 snRNP to the pre-mRNA. Splicing factor 3b is also a component of the minor U12-type spliceosome. The carboxy-terminal two-thirds of subunit 1 have 22 non-identical, tandem HEAT repeat domains that form rod-like, helical structures. Alternative splicing results in multiple transcript variants encoding different isoforms.

Relevant mutations of SF3B1 as per the present disclosure notably include mutations in a HEAT (Huntingtin, Elongation factor 3, protein phosphatase 2A, Targets of rapamycin 1) repeat domains (typically in the region corresponding to residues 622-781) and/or in the U2AF2 domain.

In some embodiments, cancer-associated mutations in SF3B1 are missense mutations within the major hotspots targeting the 5-9 heat repeat domains and notably the fifth, sixth and seventh HEAT repeats of the SF3B1 protein. These alterations affect residues that are predicted to be spatially close to one another and therefore might have a similar functional impact. (see Alsafadi, S. et al. Cancer-associated SF3B1 mutations affect alternative splicing by promoting alternative branchpoint usage. Nat. Commun. 7, 10615 (2016) and Quesada V et al., Exome sequencing identifies recurrent mutations of the splicing factor SF3B1 gene in chronic lymphocytic leukemia. Nat Genet. 2011). Example SF3B1 mutations include mutations in positions E622, Y623, R625, N626, H662, T663, K666, K700, V701, K741, G742, D984, more particularly mutations K700, E622, R625, H662, and K666. In some embodiments, mutations of SF3B1 are R625, K700 (notably K700E) and or K666.

RNA splicing involves the removal of intronic sequences from pre-mRNA and the ligation of exons to generate mature mRNA. RNA is carried out by the splicing machinery (spliceosome) composed of five snRNPs and additional proteins10. Introns contain consensus sequences that define the 5′ donor splice site (5′ss), branchpoint (BP) and 3′ acceptor splice site (3′ss), which are initially recognized by the U1 snRNP, SF1 protein and U2AF, respectively. U2AF is a heterodimer composed of U2AF2 (also known as U2AF65) and U2AF1 (also known as U2AF35), which recognize the poly-pyrimidine tract and the well-conserved AG dinucleotide sequence of 3′ss, respectively. After binding to the 3′ss, U2AF facilitates replacement of SF1 by U2 snRNP at the BP. Interaction between U1 and U2 snRNPs then triggers transesterification joining the 5′-end of the intron to the BP, most generally an adenosine located in a loosely defined consensus ˜25 nucleotides upstream of the 3′ss. The 5′ss and 3′ss are then ligated together and the branched intron is discarded (see Alsafadi, S. et al. Cancer-associated SF3B1 mutations affect alternative splicing by promoting alternative branchpoint usage. Nat. Commun. 7, 10615 (2016)). SF3B1 hotspot mutations (SF3B1^{MUT}) result in neomorphic activity causing aberrant splicing and are associated with the expression of hundreds of aberrantly spliced junctions. The most common splicing abnormality observed in SF3B1^{MUT} cells was the usage of an aberrant (or cryptic) 3′ ss (also called AG′), typically located 10 to 30 bases upstream of normal 3′ss. It has been proposed that (i) mutant SF3B1 preferentially recognizes alternative BPs upstream of the canonical sites and (ii) the alternative 3′ss used in a SF3B1^{MUT} context are less dependent on U2AF. SF3B1 hotspot mutations are typically neither gain nor loss of change-of-function mutations (see Alsafadi et al., Nat. Comm. 2016).

“Mutations that phenocopy SF3B1 mutations” (SF3B1^{MUT}) also named hereafter “SF3B1-like mutations” (SF3B1-like^{MUT}) are mutations that have the same functional consequences as SF3B1 mutations, notably major hot spots mutations of SF3B1 (SF3B1^{MHS}) as above described. Typically, SF3B1-like mutations involve usage of a cryptic 3′ slice site. In particular SF3B1-like mutations are change-of-function mutations that lead to the recognition of an alternative branchpoint upstream of the canonical BP, consequent cryptic 3′ slice usage, and an aberrant junction in a subset of mRNA defined by sequence requirements (See Alsafadi et al., Nat Comm. 2016; Darman R B et al., Cell Rep 2015 and Gozzani O et al., Mol Cell Biol 1998).

Typical examples of SF3B1-like mutations include mutations of protein(s) from the spliceosome complex, such as the SUGP1 protein.SUGP1 (SURP and G-patch domain-containing protein 1), also known as splicing factor 4 (SF4) is a protein (UniProtKB—Q81WZ8) that is encoded by the SUGP1 gene (Ensembl: ENSG00000105705) and is a spliceosomal protein which is involved in mRNA splicing. SUGP1 is recruiting the U2 small nuclear ribonucleoprotein (snRNP) in the spliceosome via the interaction of the SF3B1 HEAT domain with SUGP1. In turn SUGP1 assists in localizing U2 SNRP to the canonical branch point —BP) and 3′ splice site through direct interaction with both SF1 and U2AF2. A recent study has also shown that SF3B1 mutations disrupt SF3B1 interaction with SUGP1 in the spliceosome and that in turn SUGP1 mutations disrupting interaction with SF3B1 (in particular mutations in the G patch domain of SUGP1) reproduces mutant SF3B1 splicing defect. (Zhang et al, “Disease-causing mutations in SF3B1 alter splicing by disrupting interaction with SUGP1”, 2019 Molecular Cell 76, 1-4). Relevant SUGP1 mutations as per present definition include mutations at positions L515, G519, R625, R636 and G26, notably missense mutations L515P, G519V, R625T and P636L and the stop gain mutation G26 (see notably Liu Z, Zhang J, Sun Y, Perea-Chamblee T E, Manley J L, Rabadan R. Pan-cancer analysis identifies mutations in SUGP1 that recapitulate mutant SF3B1 splicing dysregulation [published online ahead of print, 2020 Apr. 24]. Proc Natl Acad Sci USA. 2020; 201922622. doi:10.1073/pnas.1922622117 and Alsafadi S, Dayot S, Tarin M, Houy A, Bellanger D, Cornelia M, Wassef M, Waterfall J J, Lehnert E, Roman-Roman S, Stern M H, Popova T. Genetic alterations of SUGP1 mimic mutant-SF3B1 splice pattern in lung adenocarcinoma and other cancers. Oncogene. 2021 January; 40(1):85-96. doi: 10.1038/s41388-020-01507-5).

“SF3B1 mutant associated tumors” or “SF3B1 mutant tumors” are tumors associated with mutations of SF3B1 and/or with SF3B1-like mutations, in particular mutations associated with mutation(s) of SF3B1 and/or SUGP1 as described above. By “associated with” it is herein intended that the tumor expresses the one or more SF3B1 mutant(s) and/or the one or more SF3B1-like mutant(s). In some embodiments said one or more mutations are tumor specific (i.e.: mutation(s) that are found to a level below 5%, notably below 1%, in particular that are not found in normal tissues samples).

“SF3B1 mutant associated tumors” or “SF3B1 mutant tumors” notably include notably melanoma, mucosal melanoma, mesothelioma, skin or cutaneous melanoma, uveal melanoma, orbital melanoma, hematological malignancies such as acute myeloid leukemia, chronic lymphocytic leukemia, chronic B-cell leukemia, myeloid leukemia, myeloproliferative neoplasm, myelodysplastic myeloproliferative cancer, chronic myelomonocytic leukemia or PDGFRB-associated chronic eosinophilic leukemia, renal cell carcinoma, adenoid cystic carcinoma, bladder urothelial carcinoma, liver cancer (notably hepatocellular carcinoma), lung cancer, pulmonary adenocarcinoma, pancreatic adenocarcinoma, breast cancer, and progesterone negative breast cancer. In some embodiments, the SF3B1 mutant associated tumor is uveal melanoma (UM), pulmonary adenocarcinoma, mesothelioma, liver hepatocellular carcinoma, pancreatic adenocarcinoma, breast cancer (notably unselected breast tumors and luminal breast tumors) or cutaneous melanoma, preferably, the tumor is selected from hematological malignancies, uveal melanoma and/or pulmonary adenocarcinoma.

“Growth of a tumor” or “tumor growth” according to the present disclosure relates to the tendency of a tumor to increase its size and/or to the tendency of tumor cells to proliferate.

For purposes of the present disclosure, the terms “cancer” and “cancer disease” are used interchangeably with the term “tumor” or “tumor disease”.

Cancers are classified by the type of cell that resembles the tumor and, therefore, the tissue presumed to be the origin of the tumor. These are the histology and the location, respectively.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, according to the present disclosure, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

By “treat” is meant to administer a compound or composition as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject. In particular, the term “treatment of a disease” includes curing, shortening the duration, ameliorating, preventing, slowing down or inhibiting progression or worsening, or preventing or delaying the onset of a disease or the symptoms thereof.

By “being at risk” is meant a subject, i.e. a patient, that is identified as having a higher than normal chance of developing a disease, in particular cancer, compared to the general population. In addition, a subject who has had, or who currently has, a disease, in particular cancer, is a subject who has an increased risk for developing a disease, as such a subject may continue to develop a disease. Subjects who currently have, or who have had, a cancer also have an increased risk for cancer metastases.

The therapeutically active agents or product, vaccines and compositions described herein may be administered via any conventional route, including by injection or infusion.

The agents described herein are administered in effective amounts. An “effective amount” refers to the amount which achieves a desired reaction or a desired effect alone, together with further doses, or together with further therapeutic agents. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. An effective amount of an agent described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on several of such parameters. In the case that a reaction in a patient is insufficient with an initial dose, higher doses (or effectively higher doses achieved by a different, more localized route of administration) may be used.

The pharmaceutical compositions as herein described are preferably sterile and contain an effective amount of the therapeutically active substance to generate the desired reaction or the desired effect.

The pharmaceutical compositions as herein described are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g. CpG oligonucleotides, cytokines, chemokines, saponin, GM-CSF and/or RNA and, where appropriate, other therapeutically active compounds. When used in medicine, the salts should be pharmaceutically compatible.

A “representative genome” (also known as reference genome or assembly) is a digital nucleic acid sequence data base, assembled by scientists as a representative example of species set of genes. As they are often assembled from the sequencing of DNA from a number of donors, reference genomes do not accurately represent the set of genes of any single individual (animal or person). Instead a reference provides a haploid mosaic of different DNA sequences from each donor.

A “messenger RNA (mRNA)” is a single-stranded RNA molecule that corresponds to the genetic sequence of a gene and is read by the ribosome in the process of producing a protein. mRNA is created during the process of transcription, where the enzyme RNA polymerase converts genes into primary transcript mRNA (also known as pre-mRNA). This pre-mRNA usually still contains introns, regions that will not go on to code for the final amino acid sequence. These are removed in the process of RNA splicing, leaving only exons, regions that will encode the protein. This exon sequence constitutes mature mRNA. Mature mRNA is then read by the ribosome, and, utilizing amino acids carried by transfer RNA (tRNA), the ribosome creates the peptide sequence a process called translation.

A “transcript” as herein intended is a messenger RNA (or mRNA) or a part of a mRNA which is expressed by an organism, notably in a particular tissue or even in a particular tissue. Expression of a transcript varies depending on many factors. In particular, expression of a transcript may be modified in a cancer cell as compared to a normal healthy cell.

A “transcriptome” as herein intended is the full range of messenger RNA, or mRNA, molecules expressed by an organism. The term “transcriptome” can also be used to describe the array of mRNA transcripts produced in a particular cell or tissue type. In contrast with the genome, which is characterized by its stability, the transcriptome actively changes. In fact, an organism's transcriptome varies depending on many factors, including stage of development and environmental conditions. Typically also, the transcriptome is modified in a cancer cell as compared to a corresponding normal healthy cell. Typically, the transcriptome as herein intended is the human transcriptome.

A reading frame is a way of dividing the sequence of nucleotides in a nucleic acid (DNA or RNA) molecule into a set of consecutive, non-overlapping triplets.

An open reading frame (ORF) is the part of a reading frame that has the ability to be translated into a peptide. An ORF is a continuous stretch of codons that contain a start codon (for example AUG) after the transcription starting site (TSS) and a stop codon (for example UAA, UAG or UGA). An ATG codon within the ORF (not necessarily the first) may indicate where translation starts. The transcription termination site is located after the ORF, beyond the translation stop codon. In eukaryotic genes with multiple exons, ORFs span intron/exon regions, which may be spliced together after transcription of the ORF to yield the final mRNA for protein translation.

A “canonical ORF” as herein intended is a protein coding sequence with specified reading frame within a mRNA sequence, which is described or annotated in databases such as for example Ensembl genome/transcriptome/proteome database collection (typically HG19). Typically, a canonical ORF is the annotated (in reference databases) ORF of a given exon in normal healthy cells.

A “shifted, aberrant or non-canonical ORF” as herein intended is a protein coding sequence with specified reading frame within a mRNA sequence which is shifted compared to the usual reading frame of exons in corresponding normal healthy cells. Thus, in most case non-canonical ORFs are therefore not described (i.e. unannotated) in genome databases such as for example in Ensembl genome/transcriptome/proteome database. In some embodiments however, some non-canonical (or shifted) mRNA sequences may represent minor mRNA that are expressed in normal healthy cells to a level below 5%, notably below 2%, below 1%, below 0.5%, below 0.2%, or below 0.1% of the total cell mRNA. Shifted ORF can be associated with a specific mutation and can thus be assessed by RNA seq analysis of the mutant cell as compared to a corresponding normal wild-type cell.

An exon is any part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

Thus, the untranslated sequences in 3′end and in 5′ end (3′UTR and 5′UTR) present in mature RNA after splicing are exonic sequences, but are non-coding sequences because these sequences are located upstream of the start codon for the translation (5′UTR) or downstream of the stop codon ending the translation (3′UTR).

The term “peptide or polypeptide,” is used interchangeably with “neoantigenic peptide or polypeptide” in the present specification to designate a series of residues, typically L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

“Tumor neoantigenic peptides” as per the present application are peptides that once presented by specific MHC alleles can be recognized by T cells and may induce T cell reactivity.

Typically, tumor neoantigenic peptides are entirely absent from the normal genome (in particular from the human genome). Such peptides are recognized as different from self and are presented by antigen-presenting cells (APC), such as dendritic cells (DC) and tumor cells themselves. Cross-presentation plays an important role as the APC is able to translocate exogenous antigens from the phagosome into the cytosol for proteolytic cleavage into the major histocompatibility complex I (MHC I) epitopes by the proteasome. Targeting such highly specific neoantigens (or neoantigenic peptides) enables immune cell to distinguish cancerous cells from normal cell avoiding the risk for autoimmunity. Typically, neoantigenic peptides-specific T cells possess functional avidity that may reach the avidity strength of anti-viral T cells (see Lennerz V et al., Cancer immunotherapy based on mutation-specific CD4+ T cells in human melanoma. Nat Med 2015; 21:81-5).

Typically, a tumor neoantigenic peptide may represent an abnormal or aberrant peptide that arises from consequence of epigenetic, transcriptional, translational, and post-translational alterations of tumors cells. In the present disclosure, the alterations are splicing alterations induced by a SF3B1 and/or a SF3B1-like mutation(s) in cancer cells. Typically said neoantigenic peptides are specifically expressed in tumor cells with said mutation(s). Thus, in some embodiments, the tumor neoantigenic peptides are splice variants which (i) are not expressed in the corresponding normal wild-type cell (and thus which typically have no corresponding genome/transcriptome database annotation, notably no Ensembl transcript (ENST), identifiers (IDs)), or (ii) that only represent minor species in normal wild-type cells (see above). By minor species, it is herein intended that the neoantigenic peptide is expressed at a level below 5%, notably below 2%, 1%, 0.5%, 0.2%, 0.1%, 0.05%, 0.002% of total peptide or protein expression in a cell. Thus, the tumor neoantigen peptides are typically tumor specific neoantigenic peptides (i.e.: peptides that are found to a level below 5%, notably below 1%, in particular peptides that are not found or not detected in normal tissues samples).

Typically, a subject of the present application is a mammal and notably a human. Thus typically, the representative, or reference genome or transcriptome is the human genome or transcriptome.

Unless specifically stated or obvious from context, as used herein, the term “about” is to be understood as within a range of normal tolerance in the art, for example within 2 standard deviations of the mean. About can be understood as within 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Neoantigenic Peptides, Polynucleotides and Vectors

The present disclosure relates to a tumor neoantigenic peptide, wherein said peptide:
is encoded by a part of an ORF sequence from a transcript which is associated with a SF3B1 or a SB3F1-like mutation and which is present in a SF3B1 mutant tumor sample; and
comprises at least 8, or at least 9 amino acids and binds at least one MHC molecule with an affinity of less than 500 nM; and

Typically, the peptide is a tumor specific neoantigenic peptide (it is expressed by tumor cells, notably tumor cells expressing SF3B1 mutation(s) and/or SF3B1-like mutation(s)) and preferably it is not expressed (i.e., typically detectably expressed) in normal healthy cells.

The inventors have shown that SF3B1 mutations (and typically SF3B1-like mutations) lead to aberrant open reading frames transcribed specifically in tumor cells. The inclusion of intronic sequences in mature transcripts leads to frameshifts in the following exons or less often to an in-frame insertion of few codons, eventually encoding potential neo-epitopes.

In some embodiments, according to the invention, the neoantigenic peptide comprises at least an intro-derived amino acid sequence (i.e., an amino acid sequence that is encoded by an intronic sequence) and an exon-derived amino acid sequence (i.e., an amino acid sequence that is encoded by an exonic sequence).
In some embodiments, the peptide can be encoded by a canonical ORF. In other words, the peptide is encoded by an ORF which is the ORF encoding the corresponding exon (the inclusion of intronic sequence(s) in the transcripts leads to an in-frame insertion) in normal cells (i.e., non-tumor cells, in particular in cells having no SF3B1 or SF3B1-like mutation(s) as herein described).
In other embodiments, the peptide can be encoded by a non-canonical, or shifted, ORF. In other words, the intronic sequence induces a shift of the ORF as compared to the canonical ORF of the exon.

In some embodiment, the peptide comprises only an exon-derived sequence. Typically, in such embodiments, the peptide is encoded by a non-canonical shifted ORF (due to the inclusion of intronic sequence(s) in 5′ of the exonic sequence).

Thus, in some embodiments, according to the invention:
when the neoantigenic peptide is encoded by a canonical ORF, it can typically include an exon-derived sequence and an intron-derived sequence, or be purely derived from an intronic sequence;
when the neoantigenic peptide is encoded by a non-canonical ORF, it can typically include an exon-derived sequence and an intro-derived sequence, be purely derived from an intronic sequence, or be purely derived from an exonic sequence.

By “a transcript which is associated with a SF3B1 or a SF3B1-like mutation”, it is herein intended a transcript, which expression in a cell is modified by a mutation of the SF3B1 splicing factor and/or by a SF3B1-like mutation as defined above (notably a SUGP1 mutation as defined previously). Typically, the expression in a cell of such transcript is increased. More specifically the expression of such transcript is induced by a SF3B1 or a SF3B1-like mutation (i.e.: such transcript is not expressed, not detectably expressed, or expressed below 5% in a corresponding normal cell) (see Alsafadi S et al., Nature Comm., 7:10615, 2016). Expression of transcript associated with a SF3B1 or a SF3B1-like mutation according to the present disclosure can be for example assessed by transcriptome analysis using RNA seq analysis in model cell expressing said mutation as compared to corresponding wild-type cell. Such an analysis is notably described in detail in Alsafadi S. et al., Nat Comm. 2016. The authors performed a transcriptome analysis of an uveal melanoma (UM) cohort using RNA-Seq technique. Their results showed that differential analysis of splice junctions between the SF3B1^{MUT} and SF3B1^{WT} tumours using DESeq2 (Love, Michael I et al. “Moderated estimation of fold change and dispersion for RNA-seq data with DESeq2.” Genome biology vol. 15, 12 (2014): 550. doi:10.1186/s13059-014-0550-8) revealed an overall high level of differences.

The transcript should be present in at least one tumor sample. Typically, the transcript is present in more than 10 SF3B1 mutant tumor samples, notably more than 20, 30, 40, 50, 100 or more than 200 SF3B1 mutant tumor samples, notably from UM tumor samples. Alternatively, a transcript as per the present disclosure is present in at least 30%, 40%, 50%, 60%, 70% or more of SF3B1 tumor samples, notably of UM tumor samples.

Typically, the samples are from a population of subjects. Cancer or tumor samples according to the present disclosure can be isolated from solid tumor or non-solid tumor of any of the tissues or organs as above defined.

A subject as per the present disclosure is typically a mammal, notably a human.

The peptide comprises at least 8 amino acids and is typically no more than 25, notably no more than 20 amino acids. For example the peptide may be 8-9, 8-10, 8-11, 12-25, 13-25, 12-20, or 13-20 amino acids in length and is encoded by a portion of an open reading frame (ORF) from a variant transcript sequence associated with SF3B1 or SF3B1-like mutation. The N-terminus of the peptide of at least 8 amino acids may be encoded by the triplet codon starting at any of nucleotide positions 1, 2, 3, 4, 5, 6, 7, 8, 9, 10, 11, 12, 13, 14, 15, 16, 17, 18, and higher (it being understood that the disclosure contemplates a start position that is any of the integers between 1 and 8000 without having to list every number between 1 and 8000) of the transcript sequence. In particular, the peptide may be 8-9, 8-10, 8-11, 12-25, 13-25, 12-20, or 13-20 amino acids in length.

In some embodiment the neoantigenic peptide can be selected from any one of SEQ ID NO: 1 to 1058. In some embodiment the neoantigenic peptide has at least 8, 9, 10, 11, 12, 13, 14, 15, 16, 17, 18, 19, 20, 21, 22, 23, 24 or 25 amino acids of any one of SEQ ID NO:1 to 1058.

In some embodiments, the neoantigenic peptide has at least 8 or 9 amino acids of any one of SEQ ID NO:1 to 848.

In some embodiments, the neoantigenic peptide has at least 8, 9, 10, 11, 12, 13, 14, 15, 16, 17, 18, 19, 20, 21, 22, 23, 24 or 25 amino acids of any one of SEQ ID NO:849 to 1058.

In some embodiments, a neoantigenic peptide as per the present disclosure may exhibit one or a combination of the following further characteristics:
It binds or specifically binds MHC class I of a subject and is 8 to 11 amino acids, notably 8, 9, 10, or 11 amino acids. Typically the neoantigenic peptide is 8 or 9 amino acids long, and binds to at least one MHC class I molecule of the subject.
It binds at least one HLA/MHC molecule of said subject suffering from a cancer with an affinity, sufficient for the peptide to be presented on the surface of a cell as an antigen. Typically, the neoantigenic peptide has an IC50 of less than 10⁻⁶, 10⁻⁷, 10⁻⁸, or 10⁻⁹ M; typically less than 500 nM, less than 250 nM, less than 200 nM, than 150 nM, less than 100 nM, or less than 50 nM (lower numbers indicating greater binding affinity).
It does not induce a significant autoimmune response and/or invoke immunological tolerance when administered to a subject.

In some embodiments, a tumor neoantigenic peptide as per the present disclosure binds to a MHC molecule present in at least 1%, 5%, 10%, 15%, 20%, 25% or more of subjects. Notably, a tumor neoantigenic peptide as herein disclosed is expressed in at least 1%, 5%, 10%, 15%, 20%, 25% of subjects from a population of subjects suffering from cancer More particularly, a tumor neoantigenic peptide of the present disclosure is capable of eliciting an immune response against a tumor present in at least 1%, 5%, 10%, 15%, 20%, or 25% of the subjects in the population of subjects suffering from cancer.

Such a neoantigenic peptide may be obtained in a method comprising the steps of:
identifying differential (i.e., aberrant) splice junctions by transcriptome analysis of SF3B1 mutant tumor samples from a population of subjects as compared to SF3B1 wild-type tumor samples, and
identifying ORF transcript sequences encoding for peptides having at least 8 or at least 9 amino acids;

wherein said tumor neoantigenic peptide binds to at least one Major Histocompatibility Complex (MHC) molecule of said subjects; and

wherein the transcripts have mismatches as compared with normal human transcriptome.

Identification of differential splice junctions by transcriptome analysis of SF3B1 mutant tumor samples as compared to SF3B1 wild-type tumor samples can be achieved by
mapping mRNA sequences from one or more cancer samples against a reference trancriptome,
comparing splice junctions and
selecting aberrant (e.g., unannotated) splice variants.

Said steps of identifying differential splice junctions may be carried out as described in Alsafadi S. et al., (Nature Communications 2016) which notably describes the modeling of differential junctions in SF3B1 WT as compared to SF3B1 mutant cells based on RNA seq analyses of SF3B1 mutant cells, in particular uveal melanoma cells.

Cancer cells or tumor cells according to the present disclosure can be isolated from solid tumors or non-solid tumors as previously defined. Typically, cancer cells are from uveal melanoma (UM).

RNA sequences may be obtained from solid tumors or non-solid tumors as previously defined. Typically, mRNA sequences are obtained from at least one subject, typically a population of subjects) suffering from SF3B1 mutant tumor. More particularly, mRNA sequences may be from primary or secondary tumors. In some embodiments, mRNA sequences are from metastasis from an SF3B1 mutant tumor notably metastasis from uveal melanoma (for example liver metastasis).

Open reading frame of the variant (or aberrant) transcript sequences can be then predicted using classical bioinformatics tools in the field, such as for example by using ORF finder tools which are well-known in the field (see notably https://www.ncbi.nim.nih.gov/orffinder/). The peptides encoded by a portion of an open reading frame (ORF) from a variant transcript sequence associated with a SF3B1, or a SF3B1-like mutation. They may be of 8-9, 8-10, 8-11, 12-25, 13-25, 12-20, or 13-20 amino acids in length. Typically, the N-terminus of the peptide of at least 8 amino acids can be encoded by the triplet codon starting at any of nucleotide positions 1, 2, 3, 4, 5, 6, 7, 8, 9, 10, 11, 12, 13, 14, 15, 16, 17, 18, and higher (it being understood that the disclosure contemplates a start position that is any of the integers between 1 and 8000 without having to list every number between 1 and 8000) of the transcript sequence. In particular, the peptide may be 8-9, 8-10, 8-11, 12-25, 13-25, 12-20, or 13-20 amino acids in length.

Typically the variant transcripts identified as per the method as above defined have no match on normal transcriptome from said subject(s), in other words, this means that the identified reads or sequences align to the representative genome of the subject (e.g., typically the human genome), but does not align to a reference transcriptome (i.e, from a corresponding normal healthy cell). In some embodiments, reads that align with the representative transcriptome may be discarded.

The method typically further comprises a step of determining, optionally in silico, the binding affinity of the tumor neoantigenic peptide with at least one MHC molecule of the said subject suffering from a cancer.

MHC class I proteins form a functional receptor on most nucleated cells of the body. There are 3 major MHC class I genes in HLA: HLA-A, HLA-B, HLA-C and three minor genes HLA-E, HLA-F and HLA-G. β2-microglobulin binds with major and minor gene subunits to produce a heterodimer. MHC molecules of class I consist of a heavy chain and a light chain and are capable of binding a peptide of about 8 to 11 amino acids, but usually 8 or 9 amino acids, if this peptide has suitable binding motifs, and presenting it to cytotoxic T-lymphocytes. The binding of the peptide is stabilized at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow the required flexibility. The peptide bound by the MHC molecules of class I usually originates from an endogenous protein antigen. As an example, the heavy chain of the MHC molecules of class I is typically an HLA-A, HLA-B or HLA-C monomer, and the light chain is β-2-microglobulin, in humans.

There are 3 major and 2 minor MHC class II proteins encoded by the HLA. The genes of the class II combine to form heterodimeric (αβ) protein receptors that are typically expressed on the surface of antigen-presenting cells. The peptide bound by the MHC molecules of class II usually originates from an extracellular or exogenous protein antigen. As an example, the α-chain and the β-chain are in particular HLA-DR, HLA-DQ and HLA-DP monomers, in humans. MEW class II molecules are capable of binding a peptide of about 8 to 20 amino acids, notably from 10 to 25 or from 13 to 25 if this peptide has suitable binding motifs, and presenting it to T-helper cells. These peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.

When the method is carried out on human samples, the method may comprise a step of determining the patient's class I or class I Major Histocompatibility Complex (MHC, aka human leukocyte antigen (HLA) alleles). In the present application, “MHC molecule” refers to at least one MHC class I molecule or at least one MHC Class II molecule.

A MHC allele database is carried out by analyzing known sequences of MHC I and MHC II and determining allelic variability for each domain. This can be typically determined in silico using appropriate software algorithms well-known in the field. Several tools have been developed to obtain HLA allele information from genome-wide sequencing data (whole-exome, whole-genome, and RNA sequencing data), including without limitation OptiType, Polysolver, PHLAT, HLAreporter, HLAforest, HLAminer, and seq2HLA (see Kiyotani K et al., Immunopharmacogenomics towards personalized cancer immunotherapy targeting neoantigens; Cancer Science 2018; 109:542-549). For example, the seq2hla tool (see Boegel S, Lower M, Schafer M, et al. HLA typing from RNA-Seq sequence reads. Genome Med. 2012; 4:102), which is well designed to perform the method as herein disclosed is an in silico method written in python and R, which takes standard RNA-Seq sequence reads in fastq format as input, uses a bowtie index (Langmead B, et al., Ultrafast and memory-efficient alignment of short DNA sequences to the human genome. Genome Biol. 2009, 10: R25-10.1186/gb-2009-10-3-r25) comprising all HLA alleles and outputs the most likely HLA class I and class II genotypes (in 4 digit resolution), a p-value for each call, and the expression of each class.

The affinity of all possible peptides encoded by each sequence for each MHC allele from the patient (or mouse) can be determined in silico using computational methods to predict peptide binding-affinity to HLA molecules. Indeed, accurate prediction approaches are based on artificial neural networks with predicted IC₅₀. Various software predictors may be used according to the present disclosure (see for example predictors described in Zhao W, Sher X (2018) “Systematically benchmarking peptide-MHC binding predictors: From synthetic to naturally processed epitopes”. PLoS Comput Biol 14(11): e1006457). Selection and use of such predictors are well in the field of the skilled person. In some embodiments several predictors may be used in combination.

For example, NetMHCpan software which has been modified from NetMHC to predict peptides binding to alleles for which no ligands have been reported, is well appropriate to implement the method as herein disclosed (Lundegaard C et al., NetMHC-3.0: accurate web accessible predictions of human, mouse and monkey MHC class I affinities for peptides of length 8-11; Nucleic Acids Res. 2008; 36:W509-W512; Nielsen M et al. NetMHCpan, a method for quantitative predictions of peptide binding to any HLA-A and -B locus protein of known sequence. PLoS One. 2007; 2:e796, but see also Kiyotani K et al., Immunopharmacogenomics towards personalized cancerimmunotherapy targeting neoantigens; Cancer Science 2018; 109:542-549 and Yarchoan M et al., Nat rev. cancer 2017; 17(4):209-222). NetMHCpan software predicts binding of peptides to any MHC molecule of known sequence using artificial neural networks (ANNs). The method is trained on a combination of more than 180,000 quantitative binding data and MS derived MHC eluted ligands. The binding affinity data covers 172 MHC molecules from human (HLA-A, B, C, E), mouse (H-2), cattle (BoLA), primates (Patr, Mamu, Gogo) and swine (SLA). The MS eluted ligand data covers 55 HLA and mouse alleles.

Thus in some embodiments, affinity of the selected peptide for MHC alleles is determined in silico using for example netMHCpan. in such embodiments, neoantigenic peptides bind preferably MHC class I with a binding affinity of less than 0.5% percentile rank score predicted by NetMHCpan 4.0.

Affinity may also be estimated in vitro using MHC tetramer formation assay as described in the results included therein (see example 2, point 2.1 and 2.2.2). Commercial assays for example from ImmunAware® can typically be used by the skilled person (EasYmers® kits from ImmunAware® are notably used according to their training guide). Typically, binding affinity is determined as a percentage of binding to a positive control. Generally, peptides showing a percentage of binding of at least 30%, notably at least 40% or even at least 50% of the positive control are selected. Typically, a neoantigenic peptide as per the present disclosure, and typically obtainable as per the present method, binds at least one HLA/MHC molecule with an affinity sufficient for the peptide to be presented on the surface of a cell as an antigen. Generally, the neoantigenic peptide has an IC50 affinity of less than 10⁻⁴. or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less for at least one HLA/MHC molecule (lower numbers indicating greater binding affinity), typically a molecule of a subject suffering from a cancer.

Further optional steps according to the present method may thus independently include:
a step of exclusion of predicted peptides, or aberrant junction transcripts, which are expressed in healthy cells. An alignment of the aberrant junction transcript sequence against the RNAseq data of healthy cells, typically allows determining the relative amount of aberrant junction transcript sequence(s) present in healthy cells. Typically, aberrant junction transcripts or predicted peptides expressed on healthy cells are discarded.
a step to confirm that a tumor neoantigenic peptide is not expressed in healthy cells of the subject. This step can be carried out using typically the Basic local alignment search tool (BLAST) and performing alignment of the sequence of the neoantigenic peptide against the proteome of healthy cells; Preferably, peptides that align against the proteome of normal healthy cells (for example using BLAST) are discarded.
a step to confirm that the aberrant junction transcript predicted peptide is expressed in cancer cells of the subject. The expression of the selected transcript sequence in cancer cells can be checked typically by RT-PCR in mRNA extracted from cancer cell sample.

A tumor neoantigenic peptide may first be validated by RT transcription analysis of fusion transcripts sequence in tumors cell from a subject. Typically also, immunization with a tumor neoantigenic peptide as per the present disclosure elicits a T cell response

Further assays may be achieved by recognition of naturally processed peptides by CD8 T cell killings (Sykulev, Y, Joo, M., Vturina, I., Tsomides, T J., & Eisen, H. N., Immunity 4, 565-575; 1996). For instance, T cells generated in HLA transgenic mice immunized with the reference neoantigenic peptide may be used.

T-cell based functional assays have been described in the field (see Bobisse S et al., Ann of Transl Med 2016; 4(14):262) can be carried for further validations of neoantigenic peptides of the present disclosure.

For example, peptide-MHC multimer complexes can be constructed for short peptides and their cognate HLA allele and used to interrogate patient's CD8 T cells against neoantinegic peptides (See van Rooij N et al., Tumor exome analysis reveals neoantigen-specific T cell reactivity in an ipilimumab-responsive melanoma. J Clin Oncol 2013; 19:747-52). Short and/or long peptides as well as mRNA can also be used to pulse and transduce antigen-presenting cells (APC) respectively and APC and Patient's T cells can be then co-cultured to induce the stimulation of neoantigenic peptide-specific CD4 and/or CD8 T cells. Said strategies can be typically completed by various functional assays such as IFN-γ ELISpot (see Robbins P F et al., Mining exomic sequencing data to identify mutated antigens recognized by adoptively transferred tumor-reactive T cells. Nat Med 2013; 19:747-52).

The neoantigenic peptide can also be modified by extending or decreasing the compound's amino acid sequence, e.g., by the addition or deletion of amino acids. The peptides can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions are generally made in accordance with the following Table 1 when it is desired to finely modulate the characteristics of the peptide.

TABLE 1

| Original residue | Exemplary substitution |
|---|---|
| Ala | Ser |
| Arg | Lys, His |
| Asn | Gln |
| Asp | Glu |
| Cys | Ser |
| Gln | Asn |
| Glu | Asp |
| Gly | Pro |
| His | Lys; Arg |
| Ile | Leu; Val |
| Leu | Ile; Val |
| Lys | Arg; His |
| Met | Leu; Ile |
| Phe | Tyr; Trp |
| Ser | Thr |
| Thr | Ser |
| Trp | Tyr, Phe |
| Tyr | Trp, Phe |
| Val | Ile, Leu |
| Pro | Gly |

Substantial changes in function (e.g., affinity for MHC molecules or T cell receptors) are made by selecting substitutions that are less conservative than those in above Table, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) hydrophilic residue, e.g. seryl, is substituted for (or by) a hydrophobic residue, e.g. leucyl, isoleucyl, phenylalanyl, valyl or alanyl; (b) a residue having an electropositive side chain, e.g., lysl, arginyl, or histidyl, is substituted for (or by) an electronegative residue, e.g. glutamyl or aspartyl; or (c) a residue having a bulky side chain, e.g. phenylalanine, is substituted for (or by) one not having a side chain, e.g., glycine.

The peptides and polypeptides may also comprise isosteres of two or more residues in the neoantigenic peptide or polypeptides. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

In addition, the neoantigenic peptide may be conjugated to a carrier protein, a ligand, or an antibody. Also, half-life and/or bioavailability of the peptide may be improved by PEGylation, glycosylation, polysialylation, HESylation, recombinant PEG mimetics, Fc fusion, albumin fusion, nanoparticle attachment, nanoparticulate encapsulation, cholesterol fusion, iron fusion, or acylation.

Modifications of peptides and polypeptides with various amino acid mimetics or unnatural amino acids are particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half life of the peptides of the present disclosure is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides and polypeptides may be also modified to provide desired attributes other than improved serum half-life. Indeed, in some embodiments, the peptide as herein defined is included in a polyepitopic fragment comprising the concatenation of at least two identical or different epitopes, at least one of which is an epitope as herein defined.

Multiple neoantigenic peptides (which may be identical or different) described herein can also be linked together, optionally by a spacer thus forming a multi-epitope polypeptide.

In some embodiments a multi-epitope polypeptide according to the present invention comprises one or more neoantigenic peptides as herein described. For example, such a vector can include 1 to 30, notably 5 to 25, notably 5 to 20, notably 15 to 15 polynucleotides encoding a neoantigenic peptide as herein described.

In some embodiments, the multi-epitope polypeptide comprises:
at least one neoantigenic peptide which is derived from (i.e., encoded by) a canonical ORF (in such case as above defined the neoantigenic peptide typically includes an exon-derived sequence and an intro-derived sequence or is purely derived from an intronic sequence); and/or
at least one neoantigenic peptide which is derived from (i.e., encoded by) a non-canonical, or shifted, ORF (in such case as above defined the neoantigenic peptide can include an exon-derived sequence and an intro-derived sequence, be purely derived from an exonic sequence or be purely derived from an intronic sequence).

In some embodiments, the multi-epitope polypeptide typically includes at least one neoantigenic peptide which is derived from (i.e., encoded by) a non-canonical, or shifted, ORF.

Also a polyepitopic fragment may comprise the concatenation of one or more epitopes, as herein defined (which may be identical or different), and of at least one epitope that is capable of inducing a T helper cell response.

For instance, the ability of the peptides to induce CTL activity can be enhanced by linkage to a sequence which contains at least one epitope that is capable of inducing a T helper cell response.

Particularly preferred immunogenic peptides/T helper conjugates are linked by a spacer molecule.

The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the peptide may be linked to the T helper peptide without a spacer. The neoantigenic peptide may be linked to the T helper peptide either directly or via a spacer either at the amino or carboxy terminus of the peptide. The amino terminus of either the neoantigenic peptide or the T helper peptide may be acylated. Exemplary T helper peptides include tetanus toxoid 830-843, influenza 307-319, malaria circumsporozoite 382-398 and 378-389.

Peptides, or polyepitopic fragments, as herein defined may also be in the form of a lipopeptide. Said lipopeptide is in particular obtained by addition of a lipid to an α-amino function or to a reactive function of the side chain of an amino acid of said peptide or polyepitopic fragment; it may comprise one or more chains derived from C4-C20 fatty acids, optionally branched or unsaturated (palmitic acid, oleic acid, linoleic acid, linolenic acid, 2-amino-hexadecanoic acid, pimelautide, trimexautide) or a derivative of a steroid. The preferred lipid portion is in particular represented by an Na-acetyllysine NE (palmitoyl) group, also called Ac-K (Pam).

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

A peptide according to the present disclosure can be in the form or a peptide/HLA complex. In particular, a peptide of the present disclosure can be complexed with HLA molecules, notably HLA I molecules so as to form HLA I/peptide complexes, in particular multimeric complexes such as tetramers.

In a further aspect, the present disclosure provides a nucleic acid (e.g. polynucleotide) encoding a neoantigenic peptide as herein disclosed. The nucleic acid may be selected from DNA, cDNA, PNA, CNA, RNA, either single- and/or double-stranded, or native or stabilized forms of nucleic acids, such as for example nucleic acids with a phosphorothiate backbone, or combinations thereof and it may or may not contain introns so long as it codes for the peptide. Typically, the sequence of said nucleic acid is that of the cDNA encoding said peptide or polypeptide or said fusion protein. In some embodiments, the vector comprises one or more polynucleotide sequences encoding HLA/neoantigenic complex as previously defined.

Said sequences as above defined may advantageously be modified in such a way that the codon usage is optimum in the host in which it is expressed. Only peptides that contain naturally occurring amino acid residues joined by naturally occurring peptide bonds are encodable by a polynucleotide. In some embodiments, the polynucleotide may be linked to a heterologous regulatory control sequence (e.g., heterologous transcriptional and/or translational regulatory control nucleotide sequences as well-known in the field).

A still further aspect of the disclosure provides a recombinant vector comprising a nucleic acid (or polynucleotide) as above defined. According to the present disclosure, the term “vector” is intended to mean a nucleic acid molecule capable of transporting another nucleic acid to which it is linked. One type of vector which can be used in the present invention includes, in a non-limiting manner, a linear or circular DNA or RNA molecule consisting of chromosomal, non-chromosomal, synthetic or semi-synthetic nucleic acids, such as in particular a viral vector, a plasmid or an RNA vector.

Numerous vectors into which a nucleic acid molecule of interest can be inserted in order to introduce it into and maintain it in a eukaryotic or prokaryotic host cell are known in themselves; the choice of an appropriate vector depends on the use envisioned for this vector (for example, replication of the sequence of interest, expression of this sequence, maintaining of this sequence in extrachromosomal form, or else integration into the chromosomal material of the host), and also on the nature of the host cell. For example, naked nucleic acids (DNA or RNA) or viral vectors such as adenoviruses, retroviruses, lentiviruses and AAVs, into which the sequence of interest has been previously inserted may be used; said sequence (isolated or inserted into a plasmid vector) can also be combined with a substance which allows it to cross the host cell membrane, such as a transporter, for instance a nanotransporter or a preparation of liposomes, or of cationic polymers, or else makes it possible to introduce it into said host cell using physical methods such as electroporation or microinjection. In addition, these methods can advantageously be combined, for example using electroporation combined with liposomes.

Preferably, said vector is an expression vector comprising all the elements required for the expression of a neoantigenic peptide as herein disclosed. For example, said vector comprises an expression cassette including at least one polynucleotide as defined above, under the control of appropriate heterologous regulatory sequences for transcription and optionally for translation (promoter, enhancer, intron, start codon (ATG), stop codon, polyadenylation signal, splice site) recognized by the desired host. The polynucleotide encoding the tumor neoantigenic peptide may be linked to such heterologous regulatory control nucleotide sequences or may be non-adjacent yet operably linked to such heterologous regulatory control nucleotide sequences. The vector is then introduced into the host through standard techniques. Guidance can be found for example in Sambrook et al. (1989) Molecular Cloning, A Laboratory Manual, Cold Spring Harbor Laboratory, Cold Spring Harbor, N.Y.

In some embodiments a vector according to the present invention comprises one or more polynucleotides encoding a neoantigenic peptide as herein described. For example, such a vector can include 1 to 30, notably 5 to 25, notably 5 to 20, notably 15 to 15 polynucleotides encoding a neoantigenic peptide as herein described. Typically, each polynucleotide encodes a different neoantigenic peptide.

In some embodiments, the vector comprises:
at least one polynucleotide encoding a neoantigenic peptide which is derived from (i.e., encoded by) a canonical ORF (in such case as above defined the neoantigenic peptide typically includes an exon-derived sequence and an intro-derived sequence or is purely derived from an intronic sequence); and/or
at least one polynucleotide encoding a peptide which is derived from (i.e., encoded by) a non-canonical, or shifted, ORF (in such case as above defined the neoantigenic peptide can include an exon-derived sequence and an intro-derived sequence, be purely derived from an exonic sequence or be purely derived from an intronic sequence).

In some embodiments, the vector typically includes at least one polynucleotide encoding a peptide which is derived from (i.e., encoded by) a non-canonical, or shifted, ORF.

A subject of the present disclosure is also a modified prokaryotic or eukaryotic host cell comprising a peptide, a polynucleotide or a vector as defined above, it being possible for the cell to be stably or transiently modified. The cell is in particular an antigen-presenting cell such as a dendritic cell (see also below).

Antigen Presenting Cells (APCs)

The present disclosure also encompasses a population of antigen presenting cells that have been pulsed with one or more of the neoantigenic peptides as previously defined and/or obtainable in a method as previously described. In some embodiments, the APCs have been pulsed with one or more peptides, wherein at least one of such neoantigenic peptides is encoded by a canonical ORF (i.e., the peptide is encoded by an ORF which is the ORF encoding the corresponding exon in normal cells) and/or at least one is encoded by a non-canonical, or shifted, ORF (i.e., the intronic sequence induces a shift of the ORF as compared to the canonical ORF of the exon).

Preferably, the antigen presenting cells are dendritic cell (DCs) or artificial antigen presenting cells (aAPCs) (see Neal, Lillian R et al. “The Basics of Artificial Antigen Presenting Cells in T Cell-Based Cancer Immunotherapies.” Journal of immunology research and therapy vol. 2, 1 (2017): 68-79). Dendritic cells (DC) are professional antigen-presenting cells (APC) that have an extraordinary capacity to stimulate naive T-cells and initiate primary immune responses to pathogens. Indeed, the main role of mature DCs are to sense antigens and produce mediators that activate other immune cells, particularly T cells. DCs are potent stimulators for lymphocyte activation as they express MHC molecules that trigger TCRs (signal 1) and co-stimulatory molecules (signal 2) on T cells. Additionally, DCs also secrete cytokines that support T cell expansion. T cells require presented antigen in the form of a processed peptide to recognize foreign pathogens or tumor. Presentation of peptide epitopes derived from pathogen/tumor proteins is achieved through MHC molecules. MHC class I (MHC-I) and MHC class II (MHC-II) molecules present processed peptides to CD8+ T cells and CD4+ T cells, respectively. Importantly, DCs home to inflammatory sites containing abundant T cell populations to foster an immune response. Thus, DCs can be a crucial component of any immunotherapeutic approach, as they are intimately involved with the activation of the adaptive immune response. In the context of vaccines, DC therapy can enhance T cell immune responses to a desired target in healthy volunteers or patients with infectious disease or cancer. In one embodiment, APCS are artificial APC, which are genetically modified to express the desired T-cell co-stimulatory molecules, human HLA alleles and/or cytokines. Such artificial antigen presenting cells (aAPC) are able to provide the requirements for adequate T-cell engagement, co-stimulation, as well as sustained release of cytokines that allow for controlled T-cell expansion. These cells are not subject to the constraints of time and limited availability and can be stored in small aliquots for subsequent use in generating T-cell lines from different donors, thus representing an off the shelf reagent for immunotherapy applications. Expression of potent co-stimulatory signals on these aAPC endows this system with higher efficiency lending to increased efficacy of adoptive immunotherapy. Furthermore, aAPC can be engineered to express genes directing release of specific cytokines to facilitate the preferential expansion of desirable T-cell subsets for adoptive transfer; such as long lived memory T-cells (see for review Hasan A H et al., Artificial Antigen Presenting Cells: An Off the Shelf Approach for Generation of Desirable T-Cell Populations for Broad Application of Adoptive Immunotherapy; Adv Genet Eng. 2015; 4(3): 130, Kim J V, Latouche J B, Rivière I, Sadelain M. The ABCs of artificial antigen presentation. Nat Biotechnol. 2004; 22:403-410 or Wang C, Sun W, Ye Y, Bomba H N, Gu Z. Bioengineering of Artificial Antigen Presenting Cells and Lymphoid Organs. Theranostics 2017; 7(14):3504-3516.).

Typically, the dendritic cells are autologous dendritic cells that are pulsed with a neoantigenic peptide as herein disclosed. The peptide may be any suitable peptide that gives rise to an appropriate T-cell response. The antigen-presenting cell (or stimulator cell) typically has an MHC class I or II molecule on its surface, and in one embodiment is substantially incapable of itself loading the MHC class I or II molecule with the selected antigen. The MHC class I or II molecule may readily be loaded with the selected antigen in vitro.

As an alternative the antigen presenting cell may comprise an expression construct encoding one or more tumor neoantigenic peptides as herein disclosed. Typically the APC can comprise at least one neoantigenic peptide which is encoded by a canonical ORF (i.e., the peptide is encoded by an ORF which is the ORF encoding the corresponding exon in normal cells) and/or at least one neoantigenic peptide which is encoded by a non-canonical, or shifted, ORF (i.e., the intronic sequence induces a shift of the ORF as compared to the canonical ORF of the exon).

The polynucleotide may be any suitable polynucleotide as previously defined and it is preferred that it is capable of transducing the dendritic cell, thus resulting in the presentation of a peptide and induction of immunity.

Thus the present disclosure encompasses a population of APCs than can be pulsed or loaded with the neoantigenic peptide as herein disclosed, genetically modified (via DNA or RNA transfer) to express at least one neoantigenic peptide as herein disclosed, or that comprise an expression construct encoding a tumor neoantigenic peptide of the present disclosure. Typically, the population of APCs is pulsed or loaded, modified to express, or comprises at least one, at least 5, at least 10, at least 15, or at least 20 or more different neoantigenic peptides or expression construct(s) encoding it. In some embodiments at least one of such neoantigenic peptides is encoded by a canonical ORF (i.e. the peptide is encoded by an ORF which is the ORF encoding the corresponding exon in normal cells) and/or at least one neoantigenic peptide is encoded by a non-canonical, or shifted, ORF (i.e., the intronic sequence induces a shift of the ORF as compared to the canonical ORF of the exon). The present disclosure also encompasses compositions comprising APCs as herein disclosed. APCs can be suspended in any known physiologically compatible pharmaceutical carrier, such as cell culture medium, physiological saline, phosphate-buffered saline, cell culture medium, or the like, to form a physiologically acceptable, aqueous pharmaceutical composition. Parenteral vehicles include sodium chloride solution, Ringer's dextrose, dextrose and sodium chloride, lactated Ringer's. Other substances may be added as desired such as antimicrobials. As used herein, a “carrier” refers to any substance suitable as a vehicle for delivering an APC to a suitable in vitro or in vivo site of action. As such, carriers can act as an excipient for formulation of a therapeutic or experimental reagent containing an APC. Preferred carriers are capable of maintaining an APC in a form that is capable of interacting with a T cell. Examples of such carriers include, but are not limited to water, phosphate buffered saline, saline, Ringer's solution, dextrose solution, serum-containing solutions, Hank's solution and other aqueous physiologically balanced solutions or cell culture medium. Aqueous carriers can also contain suitable auxiliary substances required to approximate the physiological conditions of the recipient, for example, enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer.

Vaccine Compositions

The present disclosure further encompasses a vaccine or immunogenic composition capable of raising a specific T-cell response comprising:
one or more neoantigenic peptides as herein defined (in the form of isolated peptides or multi-epitope polypeptides) which have optionally been modified as previously described (e.g. lipopeptide, fusion protein),
one or more nucleic acid (notably RNA) encoding one or more neoantigenic peptides as herein defined;
one or more vector as herein defined, advantageously expression vectors comprising at least one expression cassette as defined above and/or
a population of antigen presenting cells (such as autologous dendritic cells or artificial APC) as described above.

Typically, the vaccine composition comprises a pharmaceutically acceptable carrier or vehicle, a carrier substance and/or one or more adjuvants.

The pharmaceutically acceptable carriers, the carrier substances and the adjuvants are those conventionally used. Additionally, stabilizer, diluent, excipient and/or any other materials well known to those skilled in the art may be used. Such materials should be non-toxic and should not interfere with the efficacy of the active ingredient.

The carrier, or vehicle, is preferably an aqueous carrier but the precise nature of the carrier or other material will depend on the route of administration. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration.

The carrier substances are advantageously selected from the group consisting of: unilamellar or multilamellar liposomes, ISCOMs, virosomes, viral pseudoparticles, saponin micelles, solid microspheres which are saccharide (poly(lactide-co-glycolide)) or gold-bearing in nature, and nanoparticles.

The adjuvants typically increase or expand the immune response of a host to an antigenic compound. Example adjuvants include emulsifiers, muramyl dipeptides, avridine, aqueous adjuvants such as aluminum hydroxide, chitosan-based adjuvants, saponins, oils, Amphigen, LPS, bacterial cell wall extracts, bacterial DNA, CpG sequences, synthetic oligonucleotides, cytokines, squalene and combinations thereof. Emulsifier include, for example, potassium, sodium and ammonium salts of lauric and oleic acid, calcium, magnesium and aluminum salts of fatty acids, organic sulfonates such as sodium lauryl sulfate, cetyltrimethylammonium bromide, glycerylesters, polyoxyethylene glycol esters and ethers, and sorbitan fatty acid esters and their polyoxyethylene, acacia, gelatin, lecithin and/or cholesterol. Adjuvants that comprise an oil component include mineral oil, a vegetable oil, or an animal oil. Other adjuvants include Freund's Complete Adjuvant (FCA) or Freund's Incomplete Adjuvant (FIA). Cytokines useful as additional immunostimulatory agents include interferon alpha, interleukin-2 (IL-2), and granulocyte macrophage-colony stimulating factor (GM-CSF), or combinations thereof.

The compositions may further contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. See, for example, Butterfield, BMJ. 2015 22; 350 for a discussion of cancer vaccines.

In one embodiment of the present disclosure the different neoantigenic peptides, encoding polynucleotides, vectors, or APCs are selected so that one vaccine or immunogenic composition comprises peptides, encoding polynucleotides, vectors, or APCs capable of associating with different MHC molecules, such as different MHC class I molecules. Preferably, such neoantigenic peptides are capable of associating with the most frequently occurring MHC class I molecules, e.g. different fragments capable of associating with at least 2 preferred, more preferably at least 3 preferred, even more preferably at least 4 preferred MHC class I molecules. In some embodiments, the compositions comprise peptides, encoding polynucleotides, vectors, or APCs capable of associating with one or more MHC class II molecules. The MHC is optionally HLA-A, -B, -C, -DP, -DQ, or -DR.

The vaccine or immunogenic composition is capable of raising a specific cytotoxic T-cells response and/or a specific helper T-cell response.

Thus in a particular embodiment, the present disclosure also relates to a neoantigenic peptide as described above, wherein the neoantigenic peptide has a tumor specific neoepitope and is included in a vaccine or immunogenic composition in the form of a peptide, a polynucleotide encoding thereof, a vector or a population of APCs as previously defined. A vaccine composition is to be understood as meaning a composition for generating immunity for the prophylaxis and/or treatment of diseases. Accordingly, vaccines are medicines which comprise or generate antigens and are intended to be used in humans or animals for generating specific defense and protective substance by vaccination. An “immunogenic composition” is to be understood as meaning a composition that comprises or generates antigen(s) and is capable of eliciting an antigen-specific humoral or cellular immune response, e.g. T-cell response.

Pharmaceutical compositions (i.e., the vaccine or immunogenic composition) as herein described may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.”

Amounts effective for this use will depend on, e.g., the peptide composition, the manner of administration, the stage and severity of the disease being treated, the weight, age, sex and general state of health of the patient, and the judgment of the prescribing physician. For example, it may generally range for the initial immunization (that is for therapeutic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition by measuring specific CTL activity in the patient's blood.

It must be kept in mind that the peptide and compositions of the present invention may generally be employed in serious disease states, that is, life-threatening or potentially life-threatening situations, especially when the cancer has metastasized. In such cases, in view of the minimization of extraneous substances and the relative nontoxic nature of the peptide, it is possible and may be felt desirable by the treating physician to administer substantial excesses of these peptide compositions.

For therapeutic use, administration should begin at the detection or surgical removal of tumors. This is followed by boosting doses until at least symptoms are substantially abated and for a period thereafter.

The vaccine or immunogenic compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor.

Peptide Vaccines:

In some embodiments, a suitable vaccine or immunogenic composition will preferably contain between 1 and 20 neoantigenic peptides, more preferably 2, 3, 4, 5, 6, 7, 8, 9, 10, 11, 12, 13, 14, 15, 16, 17, 18, 19, 20, 21, 22, 23, 24, 25 different neoantigenic peptides, further preferred 6, 7, 8, 9, 10 11, 12, 13, or 14 different neoantigenic peptides, and most preferably 12, 13 or 14 different neoantigenic peptides. In some embodiments, at least one of the neoantigenic peptides is encoded by a canonical ORF (i.e., the peptide is encoded by an ORF which is the ORF encoding the corresponding exon in normal cells) and/or at least one is encoded by a non-canonical, or shifted, ORF (i.e., the intronic sequence induces a shift of the ORF as compared to the canonical ORF of the exon).

The neoantigenic peptide(s) may be linked to a carrier protein. Where the composition contains two or more neoantigenic peptides, the two or more (e.g. 2-25) peptides may be linearly linked by a spacer molecule as described above, e.g. a spacer comprising 2-6 nonpolar or neutral amino acids.

In a preferred embodiment, the neoantigenic peptide according to the disclosure is at least 8 or 9 residues long, notably 8 or 9 residues long, or from 13 to 25 residues long. When the peptide is less than 20 residues, in order to have a peptide better suited for in vivo immunization, said neoantigenic peptide, is optionally flanked by additional amino acids to obtain an immunization peptide of more amino acids, usually more than 20.

The concentration of peptides as herein described in the vaccine or immunogenic formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

Various particulate systems can be used for efficacious delivery of peptide vaccines. These delivery systems may also serve as adjuvants.

Typical adjuvants have been described above and include notably aluminum salts and emulsions. Emulsions are likely the most common adjuvant used in peptide cancer vaccines. They are based on a common mechanism of action of formation of a depot at the injection site that is capable of attracting the immune cells. Emulsions (see also previously) can be single (o/w, w/o) or multiple (w/o/w) (see also for review Li, Weidang et al. “Peptide Vaccine: Progress and Challenges.” Vaccines 2014; 2(3):515-536).

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

Liposomes target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871; 4,501,728; 4,837,028; and 5,019,369.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

A similar group of colloids to liposomes for the delivery of antigens include are virosomes, transfersomes, archeosomes, niosomes and cochleates. Niosomes are made of non-ionic surfactants and may be more stable than conventional liposome. Virosomes are composed of assembled viral membrane protein which render them enhanced binding to APCs and promote cytosolic delivery. Structurally, virosomes comprise 70% of naturally occurring phospholipids and 30% envelop phospholipids originating from the influenza virus. Virosomal delivery of antigens to APCS is known to enhance MHC class I and MHC class II presentation and induce both B- and T-cell responses. Virosomes are excellent adjuvant systems and are biodegradable, non-toxic, and do not induce antibodies against themselves ((see also for review Li, Weidang et al. “Peptide Vaccine: Progress and Challenges.” Vaccines 2014; 2(3):515-536).

Immunostimulatory complexes (ISCOMs) are particulate antigen delivery systems composed of antigen, cholesterol, phospholipid and saponin and around 40 nm size. ISCOMATRIX™ is a particulate adjuvant comprising cholesterol, phospholipid and saponin but without antigen. ISCOMs and ISCOMATRIX™ are composed of phospholipids as liposomes but also contain saponin adjuvant Quil A (.Review ISCOMs and ISCOMATRIX Sun H X, Xie Y, Ye Y P Vaccine. 2009 Jul. 16; 27(33):4388-401). ISCOMS can only be loaded with hydrophobic antigens. Strategies to encapsulate hydrophilic antigens into ISCOMS include: coupling of antigens to ISCOMs using amphipathic coupling protein; conjugation of hydrophilic with fatty acids and phospholipids; and, modification of protein by genetic engineering. ISCOMSs are known to induce CTL responses for native as well as modified immunogens and can mediate humoral as well as cell-mediated immune responses. (see A. Homhuan, S. Prakongpan, P. Poomvises, R. A. Maas, D. J. Crommelin, G. F. Kersten, W. Jiskoot, Eur. J. Pharm. Sci. 2004, 22, 459; M. Pearse, D. Drane, Vaccine 2004, 22, 2391; A. Coulter, R. Harris, R. Davis, D. Drane, J. Cox, D. Ryan, P. Sutton, S. Rockman, M. Pearse, Vaccine 2003, 21, 946).

Various polymers can also be used for the delivery of the vaccines. The natural polymers available for the production of nanoparticles include albumin, collagen, starch, chitosan, dextran, whereas the examples of synthetic polymers include polymethylmethacrylate, polyesters, polyanhydrides, and polyamides. Of the synthetic polyesters, polylactides (PLA), polyglycolides or polyglocolic acid (PGA) and their copolymers poly(lactide-co-glycolide) PLGA are US FDA approved for use in humans and have been tested for toxicity and safety in extensive animal studies (Key roles of adjuvants in modern vaccines. Reed S G, Orr M T, Fox C B Nat Med. 2013 December; 19(12):1597-608). The popular choice for biodegradable polymers are aliphatic polyesters such as poly(lactic acid) (PLA), poly(glycolic acid) (PGA), poly(e-caprolactone) (PCL), poly(hydroxybutyrate) (PHB) and their copolymers. Owing to their particulate nature polymeric micro and nanoparticles are known to promote uptake, transport, or presentation of antigen to APCs. They were also found to elicit both cellular and humoral immunity. The biggest advantage offered by polymer based antigen delivery systems is the sustained release (for a period of few weeks to months) of the encapsulated antigen from the polymer matrix. The rate of release of the antigens from the encapsulated polymeric particles can be controlled by the rate of degradation of the polymer matrix which, in turn, is dependent on the composition of the polymer matrix, molecular weight of the polymer and size of the particles.

Other particulate systems used to deliver vaccine antigens include carbon nanotubes, silicon dioxide nanoparticles, dendrimers (Efficient orthogonal bioconjugation of dendrimers for synthesis of bioactive nanoparticles. Gaertner H F et al, Bioconjug Chem. 2011 Jun. 15; 22(6):1103-14), ferritin nanoparticles, peptide nanocarriers, gold nanoparticles (Size-dependent impairment of cognition in mice caused by the injection of gold nanoparticles. Chen Y S et al., Nanotechnology. 2010 Dec. 3; 21(48):485102), liposome-polycation-DNA (LPD) complex (Induction of tumor-specific immunity by multi-epitope rat HER2/neu-derived peptides encapsulated in LPD Nanoparticles, Jalali S A et al, Nanomedicine. 2012 July; 8(5):692-701), oligosaccharide ester derivatives (OEDs) microparticles and combination systems, e.g., liposomes and w/o emulsion (Key roles of adjuvants in modern vaccines. Reed S G et al, Nat Med. 2013 December; 19(12):1597-608).

Peptide delivery may also include protein conjugate delivery systems such as covalent conjugation to carrier protein such as KLH (Keyhole limpet hemocyanin) (D. Miles, K. Papazisis, Clin. Breast Cancer 2003, 3(Suppl 4), S134), tetanus toxoid, the E2 core protein of the pyruvate dehydrogenase complex.

Cellular Vaccines

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and antigen presenting cell (APC) is present.

Correspondingly, it may enhance the immune response if not only the peptide is used for activation of CTLs, but if additionally, APCs with the respective MHC molecule are added. Therefore, in some embodiments the vaccine or immunogenic composition according to the present disclosure alternatively or additionally contains at least one antigen presenting cell, preferably a population of APCs.

The vaccine or immunogenic composition may thus be delivered in the form of a cell, such as an antigen presenting cell, for example as a dendritic cell vaccine. The antigen presenting cells such as a dendritic cell may be pulsed or loaded with a neoantigenic peptide as herein disclosed, may comprise an expression construct or cassette encoding a neoantigenic peptide as herein disclosed, or may be genetically modified (via DNA or RNA transfer) to express one, two or more of the herein disclosed neoantigenic peptides, for example at least 2, 3, 4, 5, 6, 7, 8, 9, or 10 neoantigenic peptides. In some embodiments, at least one of the neoantigenic peptides is encoded by a canonical ORF (i.e., the peptide is encoded by an ORF which is the ORF encoding the corresponding exon in normal cells) and/or at least one is encoded by a non-canonical, or shifted, ORF (i.e., the intronic sequence induces a shift of the ORF as compared to the canonical ORF of the exon).

DNA and RNA Vaccines

Suitable vaccines or immunogenic compositions may also be in the form of DNA or RNA relating to neoantigenic peptides as described herein. For example, DNA or RNA encoding one or more neoantigenic peptides or proteins derived therefrom may be used as the vaccine, for example by direct injection to a subject. For example, DNA or RNA encoding at least 2, 3, 4, 5, 6, 7, 8, 9, 10, 11, 12, 13, 14, 15, 16, 17, 18, 19, 20, 21, 22, 23, 24 or 25 neoantigenic peptides or proteins derived therefrom.

A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. Delivery may be achieved by nanoparticles; gene gun, microneedle array and in situ electroporation (see Hoden R B C & Stern P L, Nat. Rev. Cancer 18, 240-254 (2018) and Jorritsma, S. H. T., Gowans, E. J., Grubor-Bauk, B. & Wijesundara, D. K. Delivery methods to increase cellular uptake and immunogenicity of DNA vaccines. Vaccine 34, 5488-5494 (2016), but see also Impellizeri J A, Ciliberto G, Aurisicchio L. Electro-gene-transfer as a new tool for cancer immunotherapy in animals. Vet Comp Oncol. 2014 December; 12(4):310-8.). The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833; WO 91/06309; and Felgner et al., Proc. Natl. Acad. Sci. USA 84: 7413-7414 (1987).

RNA vaccines (see for recent techniques well-suited to the present disclosure: Diken, M., Kranz, L. M., Kreiter, S. & Sahin, U. mRNA: A versatile molecule for cancer vaccines. Curr. Issues Mol. Biol. 22, 113-128 (2017), Kranz L M et al., Systemic RNA delivery to dendritic cells exploits antiviral defence for cancer immunotherapy. Nature. 2016 Jun. 16; 534(7607):396-401; Sahin U et al., Personalized RNA mutanome vaccines mobilize poly-specific therapeutic immunity against cancer. Nature. 2017 Jul. 13; 547(7662):222-226; Beissert T et al., Improvement of In Vivo Expression of Genes Delivered by Self-Amplifying RNA Using Vaccinia Virus Immune Evasion Proteins. Hum Gene Ther. 2017 December; 28(12):1138-1146; Grabbe S et al., Translating nanoparticulate-personalized cancer vaccines into clinical applications: case study with RNA-lipoplexes for the treatment of melanoma. Nanomedicine (Lond). 2016 October; 11(20):2723-2734. Review.) may also offer advantages, as RNA cannot integrate into the genome and has therefore no oncogenic potential. Also RNA only needs to enter the cytoplasm, contrary to DNA which needs to enter the nucleus. Susceptibility to degradation may be overcome by chemical modifications and incorporation of modified nucleosides. RNA vaccines may thus comprise mRNA and/or RNA replicons (Lundstrom, K. & Replicon, R. N. A. viral vectors as vaccines. Vaccines 4, 39 (2016)). Additional delivery techniques may thus encompass condensation with protamine and encapsulation into liposomes (Lu, D., Benjamin, R., Kim, M., Conry, R. M. & Curiel, D. T. Optimization of methods to achieve mRNA-mediated transfection of tumor cells in vitro and in vivo employing cationic liposome vectors. Cancer Gene Ther. 1, 245-252 (1994)) or nanoparticles (see Wasungu, L. & Hoekstra, D. Cationic lipids, lipoplexes and intracellular delivery of genes. J. Control. Release 116, 255-264 (2006); Little, S. R. et al. Poly-β amino ester containing microparticles enhance the activity of nonviral genetic vaccines. Proc. Natl Acad. Sci. USA 101, 9534-99539 (2004); Phua, K. K. L., Leong, K. W. & Nair, S. K. Transfection efficiency and transgene expression kinetics of mRNA delivered in naked and nanoparticle format. J. Control. Release 166, 227-233 (2013); Su, X., Fricke, J., Kavanagh, D. G. & Irvine, D. J. In vitro and in vivo mRNA delivery using lipid-enveloped pH-responsive polymer nanoparticles. Mol. Pharm. 8, 774-787 (2011); Phua, K. K. L., Nair, S. K. & Leong, K. W. Messenger RNA (mRNA) nanoparticle tumour vaccination. Nanoscale 6, 7715-7729 (2014)). An overview of recent mRNA vaccine delivery systems well-suited to the present disclosure is also proposed in Grunwitz C, Kranz L M. mRNA Cancer Vaccines-Messages that Prevail. Curr Top Microbiol Immunol. 2017; 405:145-164. doi: 10.1007/82_2017_509. Review.

Delivery systems may optionally include cell-penetrating peptides, nanoparticulate encapsulation, virus like particles, liposomes, or any combination thereof. Cell penetrating peptides include TAT peptide, herpes simplex virus VP22, transportan, Antp.

In some embodiments, DNA vaccines can be delivered using nano-carriers (NC). NC are defined as particles of 1-1000 nm in size with an interfacial layer that can be composed of different materials (Nanomedicines for the treatment of hematological malignancies, Deshantri A K et al, J Control Release. 2018 Oct. 10; 287:194-215). So far, NC are used predominantly as delivery systems for drugs, adjuvants or nucleic acid-based vaccines contributing to the emerging field of nano-vaccines (DNA Nanotechnology for Precise Control over Drug Delivery and Gene Therapy, Angell C et al, Small. 2016 Mar. 2; 12(9):1117-32). Both cellular uptake and endosomal release of NC-complexed DNA is enhanced by cell penetrating peptides (CPP) which are either attached directly to DNA (Cell-penetrating peptides (CPPs): From delivery of nucleic acids and antigens to transduction of engineered nucleases for application in transgenesis, Rádis-Baptista G et al, J Biotechnol. 2017 Jun. 20; 2520:15-26) or to the DNA-complexing NC (Peptide chemistry encounters nanomedicine: recent applications and upcoming scenarios in cancer, Falanga A, Galdiero S Future Med Chem. 2018 Aug. 1; 10(16):1877-1880). NC surface and or hydrophobicity may be modified with polyethylene glycol or with moieties like antibodies or natural ligands like carbohydrates to retain CPP activity or to improve interaction with immune cell (Nanogel vaccines targeting dendritic cells: contributions of the surface decoration and vaccine cargo on cell targeting and activation, Thomann-Harwood L J et al, J Control Release. 2013 Mar. 10; 166(2):95-105).

The one or more neoantigenic peptides may also be delivered via a bacterial or viral vector containing DNA or RNA sequences which encode one or more neoantigenic peptides. The DNA or RNA may be delivered as a vector itself or within attenuated bacteria virus or live attenuated virus, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides of the invention, e.g., Salmonella typhivectors and the like, will be apparent to those skilled in the art from the description herein.

An appropriate mean of administering nucleic acids encoding the peptides as herein described involves the use of minigene constructs encoding multiple epitopes (neoantigenic peptides). Said strategy is well-suited for personalized generic cancer treatment and allow to build personalized minigene constructs comprising a selected cocktail of specific public epitopes selected for one patient or specific group of patients.

To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes. The minigene sequence can be converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides can be joined using T4 DNA ligase.

This synthetic minigene, encoding the CTL epitope polypeptide, can be also be cloned into a desired expression vector, such as plasmid DNA vectors.

Standard regulatory sequences well known to those of skill in the art are included in the vector to ensure expression in the target cells. Thus, the DNA or RNA encoding the neoantigenic peptide(s) may typically be operably linked to one or more of:
a promoter that can be used to drive nucleic acid molecule expression. AAV ITR can serve as a promoter and is advantageous for eliminating the need for an additional promoter element. For ubiquitous expression, the following promoters can be used: CMV (notably human cytomegalovirus immediate early promoter (hCMV-IE)), CAG, CBh, PGK, SV40, RSV, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA). Typically, the promoter includes a down-stream cloning site for minigene insertion. For examples of suitable promoters sequences, see notably U.S. Pat. Nos. 5,580,859 and 5,589,466.
Transcriptional transactivators or other enhancer elements, which can also increase transcription activity, e.g. the regulatory R region from the 5′ long terminal repeat (LTR) of human T-cell leukemia virus type 1 (HTLV-1) (which when combined with a CMV promoter has been shown to induce higher cellular immune response).
Translation optimizing sequences e.g. a Kozak sequence flanking the AUG initiator codon (ACCAUGG) within mRNA, and codon optimization.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immunostimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used.

In some embodiments, minigenes can be delivered using Electro-gene-transfer (EGT) (see for example Aurisicchio L et al., A novel minigene scaffold for therapeutic cancer vaccines. Oncoimmunology. 2014; 3 (1):e27529).

DNA vaccines or immunogenic compositions as herein described can be enhanced by co-delivering cytokines that promote cell-mediated immune responses, such as IL-2, IL-12, IL-18, GM-CSF and IFNγ. CXC chemokines such as IL-8, and CC chemokines such as macrophage inflammatory protein (MIP)-1α, MIP-3α, MIP-3β, and RANTES, may increase the potency of the immune response. DNA vaccine immunogenicity can also be enhanced by co-delivering plasmid-encoded cytokine-inducing molecules (e.g. LeIF), co-stimulatory and adhesion molecules, e.g. B7-1 (CD80) and/or B7-2 (CD86). Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Vaccines or immunogenic compositions comprising peptides may be administered in combination with vaccines or immunogenic compositions comprising polynucleotide encoding the peptides. For example, administration of peptide vaccine and DNA vaccine may be alternated in a prime-boost protocol. For example, priming with a peptide immunogenic composition and boosting with a DNA immunogenic composition is contemplated, as is priming with a DNA immunogenic composition and boosting with a peptide immunogenic composition.

The present disclosure also encompasses a method for producing a vaccine composition comprising the steps of:
a) Optionally, identifying at least one neoantigenic peptide according to the method as previously described;
b) producing said at least one neoantigenic peptide, at least one polypeptide encoding neoantigenic peptide(s), or at least a vector comprising said polypeptide(s) as described herein; and
c) optionally adding physiologically acceptable buffer, excipient and/or adjuvant and producing a vaccine with said at least one neoantigenic peptide, polypeptide or vector.

Another aspect of the present disclosure is a method for producing a DC vaccine, wherein said DCs present at least one neoantigenic peptide as herein disclosed.

Antibodies TCRs, CARs and Derivatives Thereof

The present disclosure also relates to an antibody or an antigen-binding fragment thereof that specifically binds a neoantigenic peptide as herein defined.

In some embodiments, the neoantigenic peptide is in association with an MHC or HLA molecule. Typically, said antibody, or antigen-binding fragment thereof binds a neoantigenic peptide as herein defined, alone or optionally in association with an MHC or HLA molecule, optionally with a Kd binding affinity of 10⁻⁷ M or less, 10⁻⁸ M or less, 10⁻⁹ M or less, 10⁻¹⁰ M or less, or 10⁻¹¹ M or less.

To promote the infiltration and recognition of tumor cells by lymphocytes T (LT), another strategy consists in using antibodies capable of recognizing more than one antigenic target simultaneously and more particularly two antigenic targets simultaneously. There are many formats of bispecific antibodies. BiTE (bi-specific T-cell engager) are the first to have been developed. These are proteins of fusion consisting of two scFvs (variable domains heavy VH and light VL chains) from two antibodies linked by a binding peptide: one recognizes the LT marker (CD3+) and the other a tumor antigen. The goal is to favor recruitment and activation of LTs in contact with tumor, thus leading to cell lysis tumor (See for review Patrick A. Baeuerle and Carsten Reinhardt; Bispecific T-Cell Engaging Antibodies for Cancer Therapy; Cancer Res 2009; 69: (12). Jun. 15, 2009; and Galaine et al., Innovations & Thérapeutiques en Oncologie, vol. 3-no 3-7, mal-août 2017).

In a particular embodiment, said antibody is a bi-specific T-cell engager that targets a tumor neoantigenic peptide as herein defined, optionally in association with a MHC or an HLA molecule and which further targets at least an immune cell antigen. Typically, the immune cell is a T cell, a NK cell or a dendritic cell. In this context, the targeted immune cell antigen may be for example CD3, CD16, CD30 or a TCR.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rlgG) fragments, variable heavy chain (VH) regions capable of specifically binding the antigen, single chain antibody fragments, including single chain variable fragments (scFv), and single domain antibodies (e.g., VHH antibodies, sdAb, sdFv, nanobody) fragments. The term encompasses genetically engineered and/or otherwise variants modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody and fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments, the antibody comprises a light chain variable domain and a heavy chain variable domain, e.g. in an scFv format.

Antibodies include variant polypeptide species that have one or more amino acid substitutions, insertions, or deletions in the native amino acid sequence, provided that the antibody retains or substantially retains its specific binding function. Conservative substitutions of amino acids are well known and described above.

The present disclosure further includes a method of producing an antibody, or antigen-binding fragment thereof, comprising a step of selecting antibodies that bind to a tumor neoantigen peptide as herein defined, optionally in association with an MHC or HLA molecule, with a Kd binding affinity of about 10⁻⁶ M or less, 10⁻⁷ M or less, 10⁻⁸ M or less, 10⁻⁹ M or less, 10⁻¹⁰ M or less, or 10⁻¹¹ M or less.

In some embodiments, the antibodies are selected from a library of human antibody sequences. In some embodiments, the antibodies are generated by immunizing an animal with a polypeptide comprising the neoantigenic peptide, optionally in association with an MHC or HLA molecule, followed by the selection step.

Antibodies including chimeric, humanized or human antibodies can be further affinity matured and selected as described above. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci.

Antibodies produced by said method, as well as immune cells expressing such antibodies or fragments thereof are also encompassed by the present disclosure.

The present disclosure also encompasses pharmaceutical compositions comprising one or more antibodies as herein disclosed alone or in combination with at least one other agent, such as a stabilizing compound, which may be administered in any sterile, biocompatible pharmaceutical carrier and optionally formulated with formulated with sterile pharmaceutically acceptable buffer(s), diluent(s), and/or excipient(s). Pharmaceutically acceptable carriers typically enhance or stabilize the composition, and/or can be used to facilitate preparation of the composition. Pharmaceutically acceptable carriers include solvents, dispersion media, coatings, antibacterial and antifungal agents, isotonic and absorption delaying agents, and the like that are physiologically compatible and, in some embodiments, pharmaceutically inert.

Administration of pharmaceutical composition comprising antibodies as herein disclosed can be accomplished orally or parenterally. Methods of parenteral delivery include topical, intra-arterial (directly to the tumor), intramuscular, spinal, subcutaneous, intramedullary, intrathecal, intraventricular, intravenous, intraperitoneal, or intranasal administration.

Thus, in addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Depending on the route of administration, the active compound, i.e., antibody, bispecific and multispecific molecule, may be coated in a material to protect the compound from the action of acids and other natural conditions that may inactivate the compound.

The composition is typically sterile and preferably fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical compositions of the disclosure can be prepared in accordance with methods well known and routinely practiced in the art. See. e.g., Remington: The Science and Practice of Pharmacy, Mack Publishing Co., 20th ed., 2000; and Sustained and Controlled Release Drug Delivery Systems, J R. Robinson, ed., Marcel Dekker, Inc., New York, 1978. Pharmaceutical compositions are preferably manufactured under GMP conditions.

The present disclosure also encompasses a T cell receptor (TCR) that targets a neoantigenic peptide as herein defined in association with an MHC or HLA molecule.

The present disclosure further includes a method of producing a TCR, or an antigen-binding fragment thereof, comprising a step of selecting TCRs that bind to a tumor neoantigen peptide as herein defined, optionally in association with an MHC or HLA molecule, optionally with a Kd binding affinity of about 10⁻⁶ M or less, 10⁻⁷ M or less, 10⁻⁸ M or less, 10⁻⁹ M or less, 10⁻¹⁰ M or less, or 10⁻¹¹ M or less.

Nucleic acid encoding the TCR can be obtained from a variety of sources, such as by polymerase chain reaction (PCR) amplification of naturally occurring TCR DNA sequences, followed by expression of antibody variable regions, followed by the selecting step described above. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

A “T cell receptor” or “TCR” refers to a molecule that contains a variable a and β chains (also known as TCRa and TCRp, respectively) or a variable γ and δ chains (also known as TCRy and TCR5, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains {e.g., a-chain, β-chain) can contain two immunoglobulin domains, a variable domain (e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., a-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD3ζ chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

While T-cell receptors (TCRs) are transmembrane proteins and do not naturally exist in soluble form, antibodies can be secreted as well as membrane bound. Importantly, TCRs have the advantage over antibodies that they in principle can recognize peptides generated from all degraded cellular proteins, both intra- and extracellular, when presented in the context of MHC molecules. Thus TCRs have important therapeutic potential.

The present disclosure also relates to soluble T-cell receptors (sTCRs) that contain the antigen recognition part directed against a tumor neoantigenic peptide as herein disclosed (see notably Walseng E, Wälchli S, Fallang L-E, Yang W, Vefferstad A, Areffard A, et al. (2015) Soluble T-Cell Receptors Produced in Human Cells for Targeted Delivery. PLoS ONE 10(4): e0119559). In a particular embodiment, the soluble TCR can be fused to an antibody fragment directed to a T cell antigen, optionally wherein the targeted antigen is CD3 or CD16 (see for example Boudousquie, Caroline et al. “Polyfunctional response by ImmTAC (IMCgp100) redirected CD8+ and CD4+ T cells.” Immunology vol. 152, 3 (2017): 425-438. doi:10.1111/imm.12779). Examples of TCRs with specific CDR3 in their alpha/beta variable regions according to the present application are notably illustrated in table 3 (see the results section). Thus, the present application encompasses TCRs comprising at least a CDR3 of the alpha variable region and/or of the beta variable region as exemplified in table 3. In some embodiments encompassed TCRs comprised an alpha CDR3 and a beta CDR3 as exemplified in table 3.

The present disclosure also encompasses a chimeric antigen receptor (CAR) which is directed against a tumor neoantigenic peptide as herein disclosed. CARs are fusion proteins comprising an antigen-binding domain, typically derived from an antibody, linked to the signalling domain of the TCR complex. CARs can be used to direct immune cells such T-cells or NK cells against a tumor neoantigenic peptide as previously defined with a suitable antigen-binding domain selected.

The antigen-binding domain of a CAR is typically based on a scFv (single chain variable fragment) derived from an antibody. In addition to an N-terminal, extracellular antibody-binding domain, CARs typically may comprise a hinge domain, which functions as a spacer to extend the antigen-binding domain away from the plasma membrane of the immune effector cell on which it is expressed, a transmembrane (TM) domain, an intracellular signalling domain (e.g. the signalling domain from the zeta chain of the CD3 molecule (CD3) of the TCR complex, or an equivalent) and optionally one or more co-stimulatory domains which may assist in signalling or functionality of the cell expressing the CAR. Signalling domains from co-stimulatory molecules including CD28, OX-40 (CD134), and 4-1BB (CD137) can be added alone (second generation) or in combination (third generation) to enhance survival and increase proliferation of CAR modified T cells. Potential co-stimulatory domains also include ICOS-1, CD27, GITR, DAP10, and CD28.

Thus, the CAR may include
(1) In its extracellular portion, one or more antigen binding molecules, such as one or more antigen-binding fragment, domain, or portion of an antibody, or one or more antibody variable domains, and/or antibody molecules.
(2) In its transmembrane portion, a transmembrane domain derived from human T cell receptor-alpha or -beta chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR. In some embodiments, the transmembrane domain is derived from CD28, CD8 or CD3-zeta.
(3) One or more co-stimulatory domains, such as co-stimulatory domains derived from human CD28, 4-1BB (CD137), ICOS-1, CD27, OX 40 (CD137), DAP10, and GITR (AITR). In some embodiments, the CAR comprises co-stimulating domains of both CD28 and 4-1BB.
(4) In its intracellular signalling domain, an intracellular signalling domain comprising one or more ITAMs, for example, the intracellular signalling domain is CD3-zeta, or a variant thereof lacking one or two ITAMs (e.g. ITAM3 and ITAM2), or the intracellular signalling domain is derived from FcεRIγ.

The CAR can be designed to recognize tumor neoantigenic peptide alone or in association with an HLA or MHC molecule.

Exemplary antigen receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Pat. No. 7,446,190, and those described in International Patent Application Publication No.:

The present disclosure also encompasses polynucleotides encoding antibodies, antigen-binding fragments or derivatives thereof, TCRs and CARs as previously described as well as vector comprising said polynucleotide(s).

Immune Cells

The present disclosure further encompasses immune cells which target one or more tumor neoantigenic peptides as previously described.

As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, the term “T cell” includes cells bearing a T cell receptor (TCR), in particular TCR directed against a tumor neoantigenic peptide as herein disclosed. T-cells according to the present disclosure can be selected from the group consisting of inflammatory T-lymphocytes, cytotoxic T-lymphocytes, regulatory T-lymphocytes, Mucosal-Associated Invariant T cells (MAIT), Yδ T cell, tumour infiltrating lymphocyte (TILs) or helper T-lymphocytes included both type 1 and 2 helper T cells and Th17 helper cells. In another embodiment, said cell can be derived from the group consisting of CD4+ T-lymphocytes and CD8+ T-lymphocytes. Said immune cells may originate from a healthy donor or from a subject suffering from a cancer.

Immune cells can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person, such as FICOLL™ separation. In one embodiment, cells from the circulating blood of a subject are obtained by apheresis. In certain embodiments, T-cells are isolated from PBMCs. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes, for instance by using CD14 DYNABEADS®. In some embodiments, red blood cells may be lysed prior to the density gradient centrifugation.

In another embodiment, said cell can be derived from a healthy donor, from a subject diagnosed with cancer. The cell can be autologous or allogeneic.

In allogeneic immune cell therapy, immune cells are collected from healthy donors, rather than the patient. Typically these are HLA matched to reduce the likelihood of graft vs. host disease. Alternatively, universal ‘off the shelf’ products that may not require HLA matching comprise modifications designed to reduce graft vs. host disease, such as disruption or removal of the TCRαβ receptor. See Graham et al., Cells. 2018 October; 7(10): 155 for a review. Because a single gene encodes the alpha chain (TRAC) rather than the two genes encoding the beta chain, the TRAC locus is a typical target for removing or disrupting TCRαβ receptor expression. Alternatively, inhibitors of TCRαβ signalling may be expressed, e.g. truncated forms of CD3 can act as a TCR inhibitory molecule. Disruption or removal of HLA class I molecules has also been employed. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression. Ren et al. simultaneously knocked out TCRαβ, B2M and the immune-checkpoint PD1. Generally, the immune cells are activated and expanded to be utilized in the adoptive cell therapy. The immune cells as herein disclosed can be expanded in vivo or ex vivo. The immune cells, in particular T-cells can be activated and expanded generally using methods known in the art. Generally the T-cells are expanded by contact with a surface having attached thereto an agent that stimulates a CD3/TCR complex associated signal and a ligand that stimulates a co-stimulatory molecule on the surface of the T cells.

In one embodiment of the present disclosure, the immune cell can be modified to be directed to tumor neoantigenic peptides as previously defined. In a particular embodiment, said immune cell may express a recombinant antigen receptor directed to said neoantigenic peptide its cell surface. By “recombinant” is meant an antigen receptor which is not encoded by the cell in its native state, i.e. it is heterologous, non-endogenous. Expression of the recombinant antigen receptor can thus be seen to introduce new antigen specificity to the immune cell, causing the cell to recognise and bind a previously described peptide. The antigen receptor may be isolated from any useful source. In some embodiments, the cells comprise one or more nucleic acids introduced via genetic engineering that encode one or more antigen receptors, wherein the antigen include at least one tumor neoantigenic peptide as per the present disclosure.

Among the antigen receptors as per the present disclosure are genetically engineered T cell receptors (TCRs) and components thereof, as well as functional non-TCR antigen receptors, such as chimeric antigen receptors (CAR) as previously described.

Methods by which immune cells can be genetically modified to express a recombinant antigen receptor are well known in the art. A nucleic acid molecule encoding the antigen receptor may be introduced into the cell in the form of e.g. a vector, or any other suitable nucleic acid construct. Vectors, and their required components, are well known in the art. Nucleic acid molecules encoding antigen receptors can be generated using any method known in the art, e.g. molecular cloning using PCR. Antigen receptor sequences can be modified using commonly-used methods, such as site-directed mutagenesis.

The present disclosure also relates to a method for providing a T cell population which targets a tumor neoantigenic peptide as herein disclosed.

The T cell population may comprise CD8+ T cells, CD4+ T cells or CD8+ and CD4+ T cells. T cell populations produced in accordance with the present disclosure may be enriched with T cells that are specific to, i.e. target, the tumor neoantigenic peptide of the present disclosure. That is, the T cell population that is produced in accordance with the present disclosure will have an increased number of T cells that target one or more tumor neoantigenic peptide. For example, the T cell population of the disclosure will have an increased number of T cells that target a tumor neoantigenic peptide compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a tumor neoantigenic peptide will be increased.

T cell populations produced in accordance with the present disclosure may be enriched with T cells that are specific to, i.e. target, tumor neoantigenic peptide. That is, the T cell population that is produced in accordance with the present disclosure will have an increased number of T cells that target one or more tumor neoantigenic peptide of the present disclosure. For example, the T cell population of the present disclosure will have an increased number of T cells that target a tumor neoantigenic peptide compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a tumor neoantigenic peptide will be increased.

The T cell population according to the present disclosure may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a tumor neoantigenic peptide as herein disclosed. For example, the T cell population may have about 0.2%-5%, 5%-10%, 10-20%, 20-30%, 30-40%, 40-50%, 50-70% or 70-100% T cells that target a tumor neoantigenic peptide of the present disclosure.

An expanded population of tumor neoantigenic peptide-reactive T cells may have a higher activity than a population of T cells not expanded, for example, using a tumor neoantigenic peptide. Reference to “activity” may represent the response of the T cell population to restimulation with a tumor neoantigenic peptide, e.g. a peptide corresponding to the peptide used for expansion, or a mix of tumor neoantigenic peptide. Suitable methods for assaying the response are known in the art. For example, cytokine production may be measured (e.g. IL2 or IFNy production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

In a preferred embodiment present disclosure provides a plurality or population, i.e. more than one, of T cells wherein the plurality of T cells comprises a T cell which recognizes a clonal tumor neoantigenic peptide and a T cell which recognizes a different clonal tumor neoantigenic peptide. As such, the present disclosure provides a plurality of T cells which recognize different clonal tumor neoantigenic peptide. Different T cells in the plurality or population may alternatively have different TCRs which recognize the same tumor neoantigenic peptide.

In a preferred embodiment the number of clonal tumor neoantigenic peptide recognized by the plurality of T cells is from 2 to 1000. For example, the number of clonal neo-antigens recognized may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, preferably 2 to 100. There may be a plurality of T cells with different TCRs but which recognize the same clonal neo-antigen.

The T cell population may be all or primarily composed of CD8+ T cells, or all or primarily composed of a mixture of CD8+ T cells and CD4+ T cells or all or primarily composed of CD4+ T cells.

In particular embodiments, the T cell population is generated from T cells isolated from a subject with a tumor. For example, the T cell population may be generated from T cells in a sample isolated from a subject with a tumor. The sample may be a tumor sample, a peripheral blood sample or a sample from other tissues of the subject.

In a particular embodiment the T cell population is generated from a sample from the tumor in which the tumor neoantigenic peptide is identified. In other words, the T cell population is isolated from a sample derived from the tumor of a patient to be treated. Such T cells are referred to herein as ‘tumor infiltrating lymphocytes’ (TILs).

T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient.

Cancer Therapeutic Methods

In any of the embodiments, the Cancer Therapeutic Products described herein may be used in methods for inhibiting proliferation of cancer cells. The Cancer Therapeutic Products described herein may also be used in the treatment of cancer, in patients suffering from cancer, or for the prophylactic treatment of cancer, in patients at risk of cancer.

Cancers that can be treated using the therapy described herein include any solid or non-solid tumors associated with SF3B1 or SF3B1-like mutation(s) as previously defined, more particularly with SF3B1 or SUGP1 mutations as defined above. Of particular interest according to the present disclosure is uveal melanoma.

The therapy described herein is also applicable to the treatment of patients in need thereof who have not been previously treated.

A subject as per the present disclosure is typically a patient in need thereof that has been diagnosed with cancer or is at risk of developing cancer. The subject is a mammal, typically a human. In some embodiments, the patient is a metastatic patient notably a metastatic patient suffering from uveal melanoma.

The present disclosure also pertains to a neoantigenic peptide, a population of APCs, a vaccine or immunogenic composition, a polynucleotide encoding a neoantigenic peptide or a vector as previously defined for use in cancer vaccination therapy of a subject or for treating cancer in a subject, wherein the peptide(s) binds at least one MHC molecule of said subject.

The present disclosure also provides a method for treating cancer in a subject comprising administering a vaccine or immunogenic composition as described herein to said subject in a therapeutically effective amount to treat the subject. The method may additionally comprise the step of identifying a subject who has cancer.

The present disclosure also relates to a method of treating cancer comprising producing an antibody or antigen-binding fragment thereof by the method as herein described and administering to a subject with cancer said antibody or antigen-binding fragment thereof, or with an immune cell expressing said antibody or antigen-binding fragment thereof, in a therapeutically effective amount to treat said subject.

The present disclosure also relates to an antibody (including variants and derivatives thereof), a T cell receptor (TCR) (including variants and derivatives thereof), or a CAR (including variants and derivatives thereof) which are directed against a tumor neoantigenic peptide as herein described, optionally in association with an MHC or HLA molecule, for use in cancer therapy of a subject, wherein the tumor neoantigenic peptide binds at least one MHC molecule of said subject.

The present disclosure also relates to an antibody (including variants and derivatives thereof), a T cell receptor (TCR) (including variants and derivatives thereof), or a CAR (including variants and derivatives thereof) which are directed against a tumor neoantigenic peptide as herein described, optionally in association with an MHC or HLA molecule, or an immune cell which targets a neoantigenic peptide, as previously defined, for use in adoptive cell or CAR—T cell therapy in a subject, wherein the tumor neoantigenic peptide binds at least one MHC molecule of said subject.

Typically, the skilled person is able to select an appropriate antigen receptor which binds and recognizes a tumor neoantigenic peptide as previously defined with which to redirect an immune cell to be used for use in cancer cell therapy. In a particular embodiment, the immune cell for use in the method of the present disclosure is a redirected T-cell, e.g. a redirected CD8+ and/or CD4+ T-cell.

In some embodiments, cancer treatment, vaccination therapy and/or adoptive cell cancer therapy as above described are administered in combination with additional cancer therapies. In particular, the T cell compositions according to the present disclosure may be administered in combination with checkpoint blockade therapy, co-stimulatory antibodies, chemotherapy and/or radiotherapy, targeted therapy or monoclonal antibody therapy.

Checkpoint inhibitors include, but are not limited to, PD-1 inhibitors, PD-L1 inhibitors, Lag-3 inhibitors, Tim-3 inhibitors, TIGIT inhibitors, BTLA inhibitors, V-domain Ig suppressor of T-cell activation (VISTA) inhibitors and CTLA-4 inhibitors, IDO inhibitors for example. Co-stimulatory antibodies deliver positive signals through immune-regulatory receptors including but not limited to ICOS, CD137, CD27 OX-40 and GITR. In a preferred embodiment the checkpoint inhibitor is a CTLA-4 inhibitor.

A chemotherapeutic entity as used herein refers to an entity which is destructive to a cell, that is the entity reduces the viability of the cell. The chemotherapeutic entity may be a cytotoxic drug.

A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNa, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,ρ′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

“In combination” may refer to administration of the additional therapy before, at the same time as or after administration of the T cell composition according to the present disclosure.

In addition or as an alternative to the combination with checkpoint blockade, the T cell composition of the present disclosure may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells including but not limited to PD-1, Lag-3, Tim-3, TIGIT, BTLA CTLA-4 and combinations of these. The T cell as discussed here may be modified by any of these methods.

The T cell according to the present disclosure may also be genetically modified to express molecules increasing homing into tumours and or to deliver inflammatory mediators into the tumour microenvironment, including but not limited to cytokines, soluble immune-regulatory receptors and/or ligands.

In a particular embodiment, said tumor neoantigenic peptide is used in cancer vaccination therapy in combination with another immunotherapy such as immune checkpoint therapy, more particularly in combination with antibodies anti-PD1, anti-PDL1, anti-CTLA-4, anti-TIM-3, anti-LAG3, anti-GITR.

### BRIEF DESCRIPTION OF THE DRAWINGS

FIG. 1: SF3B1-like splicing pattern and SUGP1 in the TCGA cohorts a. Screening of the whole set of RNA-seq data in TCGA using the SBT score. The SBT score (the occurrence of 1,443 aberrant splice junctions in fastq RNA-seq) for each sample is plotted (x-axis) against the size of RNA-seq bam file (y-axis). Cases with SF3B1 hotspot and other mutations are indicated. The linear trend and the cutoff lines for the cases further explored are shown (dash and red lines).

b. Principal component analysis of the selected 449 cases (including cases with high SBT scores, cases with mutations in splicing factors SF3B1, SRSF2 and U2AF1, and tissue-matched control tumor cases) characterized by the ratio of aberrant to canonical 3′ss expression in top 400 cryptic 3′ss junctions selected in an unsupervised way (see Methods). Cases with SUGP1 alterations are highlighted (magenta spots). The two first principal components, PC1 and PC2 are plotted on x and y-axes, respectively. The fraction of variance explained by each principal component is indicated.

c. Hierarchical clustering and the heatmap of differentially spliced junctions in the LUAD cohort. Cases are denoted according to their alterations in SF3B1 or SUGP1. For comparison, gene expression level of the junction clustering is shown (differential expression of junctions is not the consequence of differential gene expression).

d. Distances between the cryptic and canonical 3′ss in the LUAD cohort for the top differentially expressed junctions in SF3B1 mutated and SUGP1 altered cases. The position of the canonical 3′ss is set to 0.

FIG. 2: Effect of SUGP1 knockdown and 3 different mutations on splicing in 181 HEK293T cells.

a. Effect of siRNA-mediated knockdown of SUGP1, on the aberrant splice forms of DPH5, DLST and ARMC9 in HEK293T cell line. Relative expression of cryptic 3′ss junction normalized to the canonical 3′ss junction was determined by quantitative RT-PCR, and effect of the different siRNA #1,3,6 and 21 was compared with the control (CTL) (Paired t-test; *, p<0.05; **, p<0.005; ***, p<0.0005). The protein knockdown was confirmed by immunoblotting with anti-SUGP1, using β-actin as a loading control.

b. Effect of siRNA-mediated knockdown of SUGP1, overexpression of wild-type SUGP1 or SF3B1, overexpression of SUGP1^{L515P, R625T or P636L} or SF3B1^{K700E} on the aberrant splice form of DPH5 in HEK293T cell line. Relative expression of cryptic 3′ss junction normalized to the canonical 3′ss junction of DPH5 was determined by quantitative RT-PCR. The results are average of three replicates and are represented as mean±sd, and each condition is compared to the control (Paired t-test;*, p<0.05; **, p<0.005). The protein knockdown or overexpression was confirmed by immunoblotting with anti-Flag and anti-SUGP1 using β-actin as a loading control.

c. 2-D plot of the differential aberrant splice junction expression in transiently SUGP1-depleted HEK293T cells (y-axis) and cells overexpressing SF3B1K700E (x-axis), as measured by RNA-seq.

d. Minigene splice assay of two SF3B1^{MUT}-sensitive 3′ (ENOSF1, TMEM14C) and their cryptic (BP′) and canonical (BP) branchpoint mutants. Gel electrophoresis shows the different splicing processes for minigene ExonTrap constructions in SF3B1^{WT} cell line HEK293T with or without siRNA-mediated knockdown of SUGP1. The lower band corresponds to the usage of the canonical 3′ss. The intermediate band corresponds to the usage of the cryptic 3′ss. The upper band corresponds to the heteroduplex formation from the two products.

FIG. 3: SF3B1^{MHS}-like splice pattern analysis in HAP1^{SUGP1-P636L} isogenic cell line.

a. Hierarchical clustering and heatmap analysis of differential splice junctions in HAP1 and HAP1SUGP1-P636L isogenic cell lines. Three biological replicates for each cell lines (R1-R3) were analyzed by RNA-seq. Below the array tree and the subtype identification row, the heatmap of the differential splice junctions is shown. The corresponding gene level expression heatmap is shown (right panel).

b. Barplot representing the expressed aberrant splicing events in HAP1 and HAP1SUGP1-P636L. A3SS: junctions with alternative 3′ splice site; A5SS: junctions with alternative 5′ splice site; MXE: junctions with alternative 3′SS and 5′SS; RI: intron retention; SE: exon skipping.

c. Distances between the cryptic and canonical 3′ss in HAP1 and HAP1^{SUGP1-P636L} isogenic cell lines. For cryptic 3′ss within the 50 nts preceding the canonical 3′ss, the distance between the cryptic and corresponding canonical 3′ss was plotted as a histogram. The position of the canonical 3′ss is set to 0.

d. siRNA-mediated knockdown of SUGP1^{WT} and SUGP1^{P636L} impact on DPH5 aberrant junction expression in HAP1 isogenic cell lines. Relative expression of cryptic 3′ss junction normalized to the canonical 3′ss junction of DPH5 was determined by quantitative RT-PCR. The results are average of three replicates and are represented as mean±s.d, and each condition is compared to the control (Paired t test; **, p<0.005; ***, p<0.0005).

FIG. 4: SF3B1^{mut} related neo-epitope prediction. (A) Expression of selected alternative spliced mRNA forms in one SF3B1^{mut} and two SF3B1^{wt} UM xenografts estimated by fragment length quantitation. (B) Bioinformatics pipeline to predict candidate neoepitopes. (C) Candidate neoepitopes prediction for the most frequent MHC-I alleles. Rank frequency was determined from a set of 400,000 random peptides (12). Strong binders are defined by a rank <0.5% and are squared for HLA-A*0201.

FIG. 5. Characterization of SF3B1^{mut}-related neo-epitope specific CD8 T cells found in the blood of metastatic uveal melanoma (mUM) patients.

(A) Identification of CD8⁺ T cells recognizing the indicated specificities using HLA-A2:peptide tetramers labelled with two different fluorochomes. Representative staining enabling to divide the Tet⁺CD8⁺ cells into naive (CCR7⁺ECD45RA⁺), central memory (CCR7⁺CD45RA⁻), effector-memory (CCR7⁻CD45RA⁻) and effector-memory (CCR7⁻CD45RA⁺). Non-naïve CD8⁺ T cells specific for Melan-A are only found in UM patients whereas A2:37-specific non-naïve CD8⁺ T cells are only found in SF3B1^{mut} patients. (B) Frequency of the indicated specificities in CD8⁺ cells from the blood of patients with SF3B1^{mut} tumors. (C) Each dot corresponds to the proportion of the CD8⁺ T cells that are not naive in healthy donors (n=4), SF3B1^{mut} (n=8) or SF3B1^{wt} (n=5) patients. Dots on the X axis correspond to a number of Ter⁺ cells below 10.

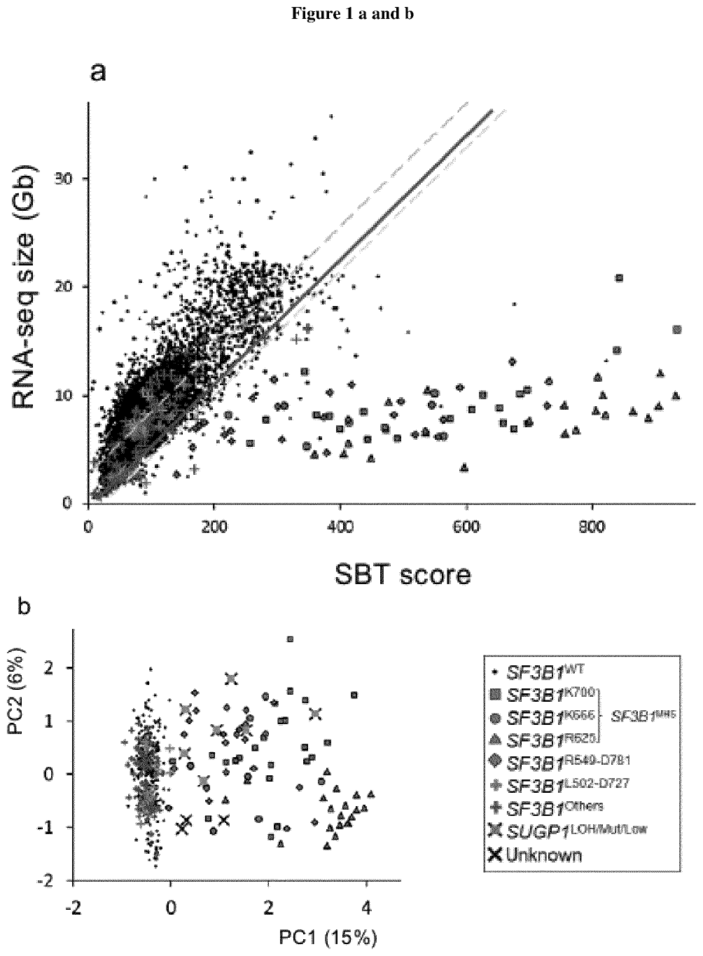

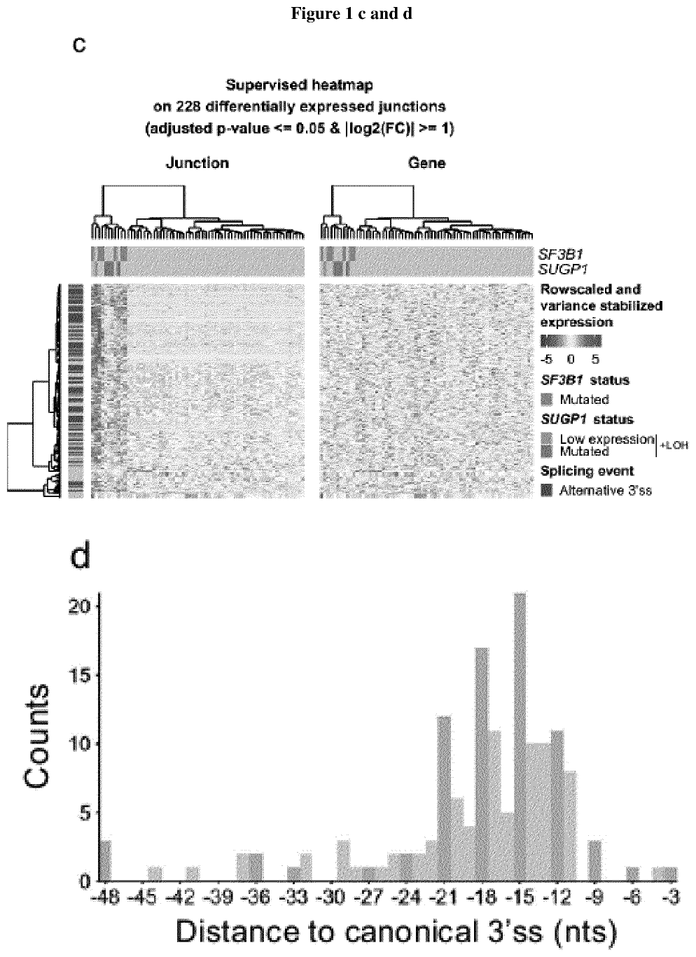

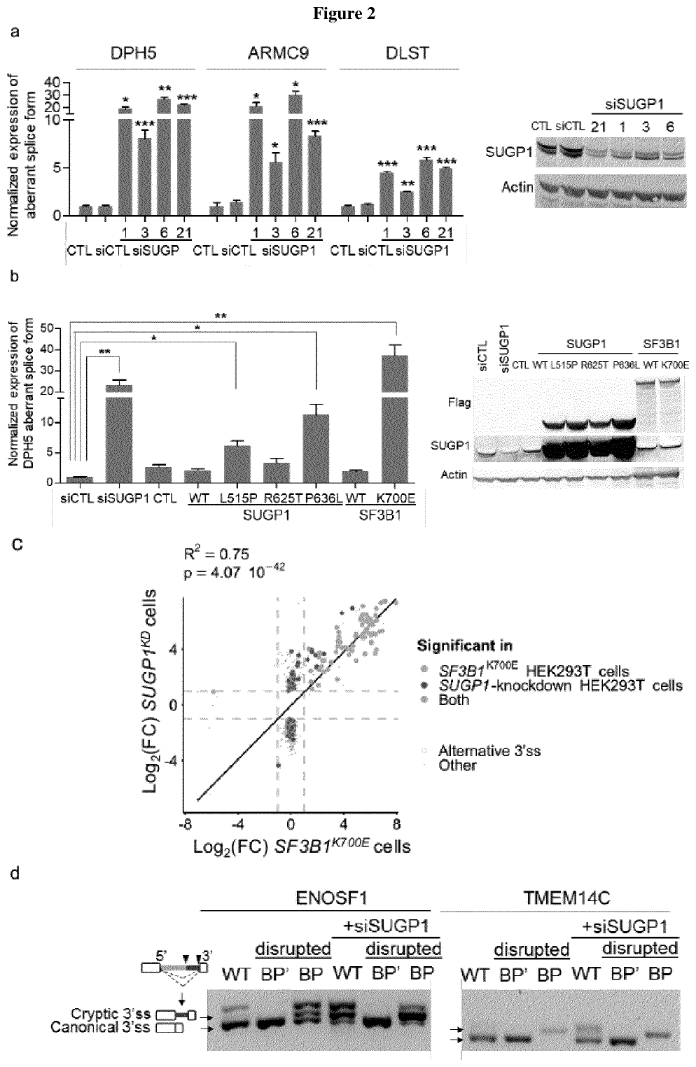

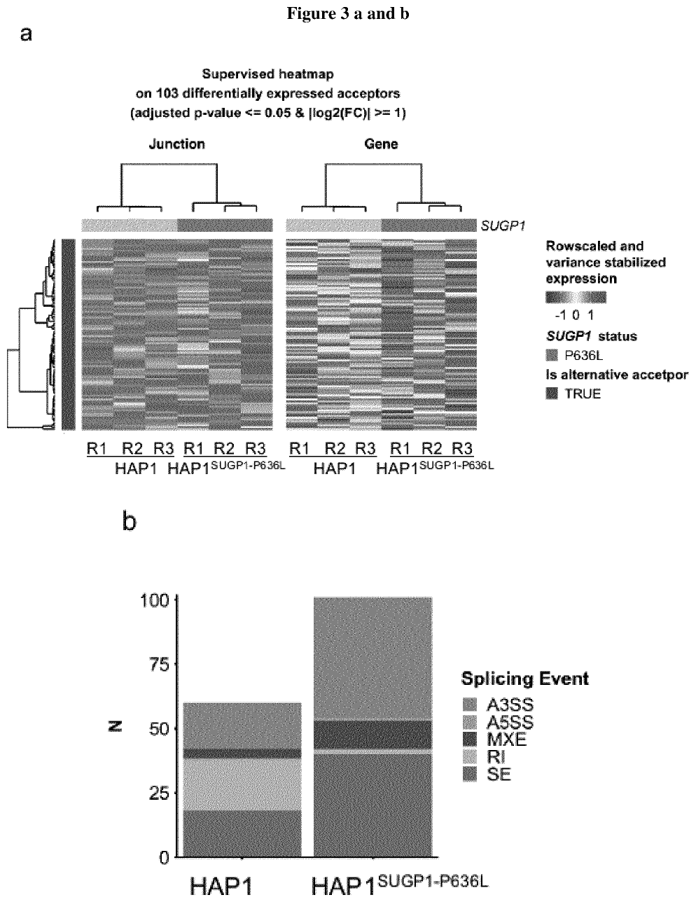

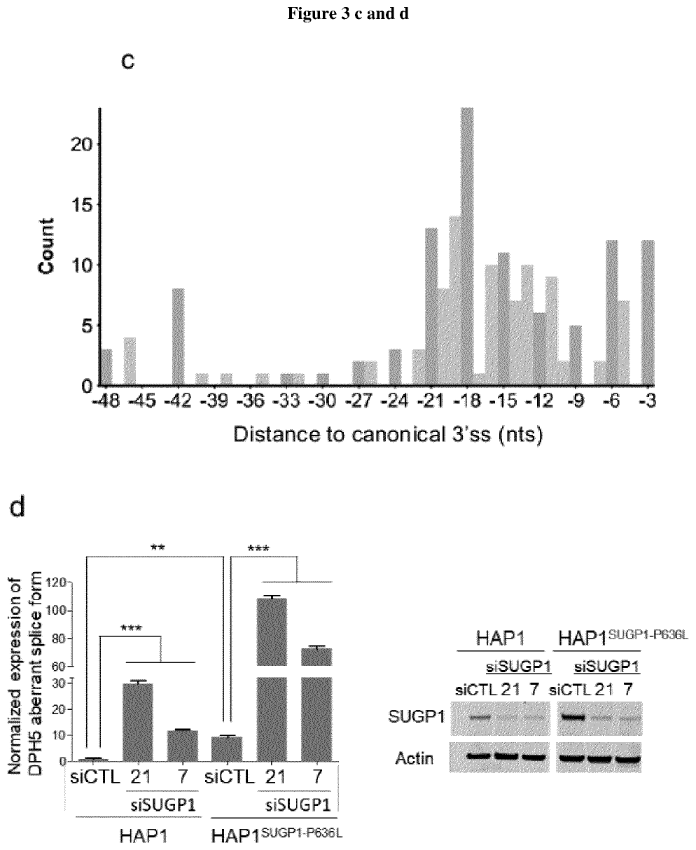

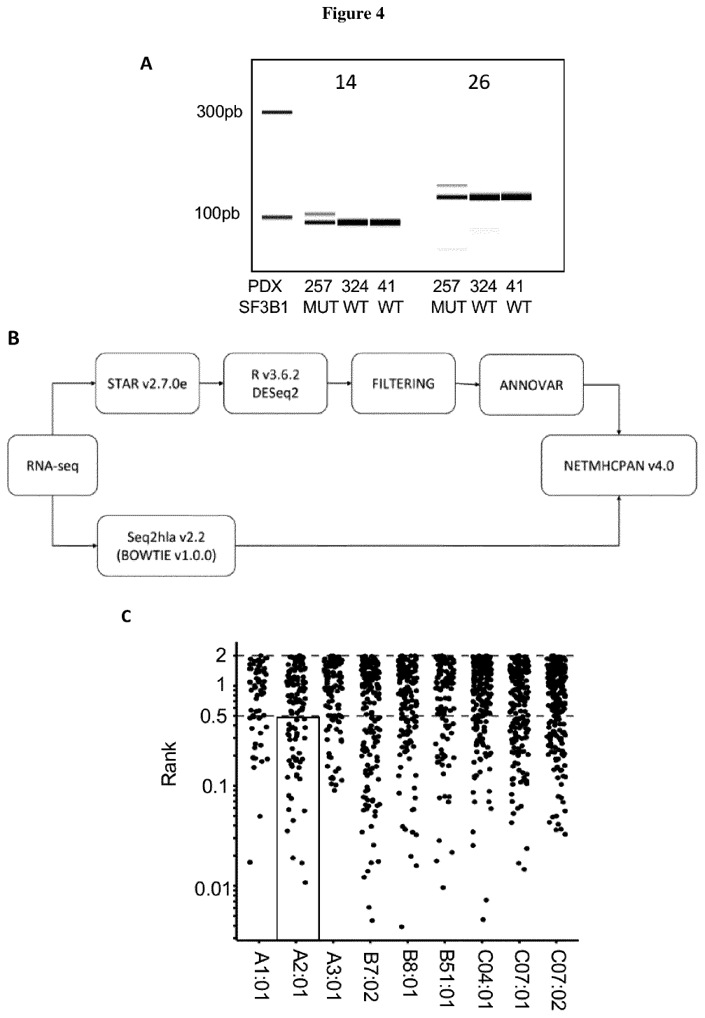

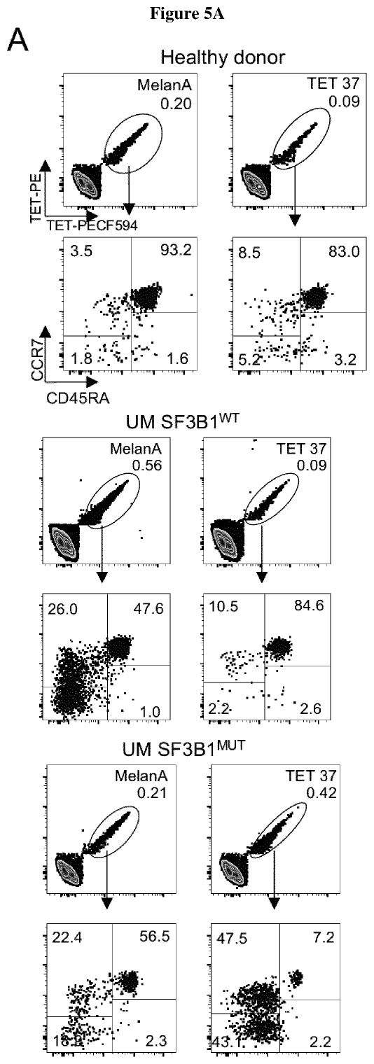

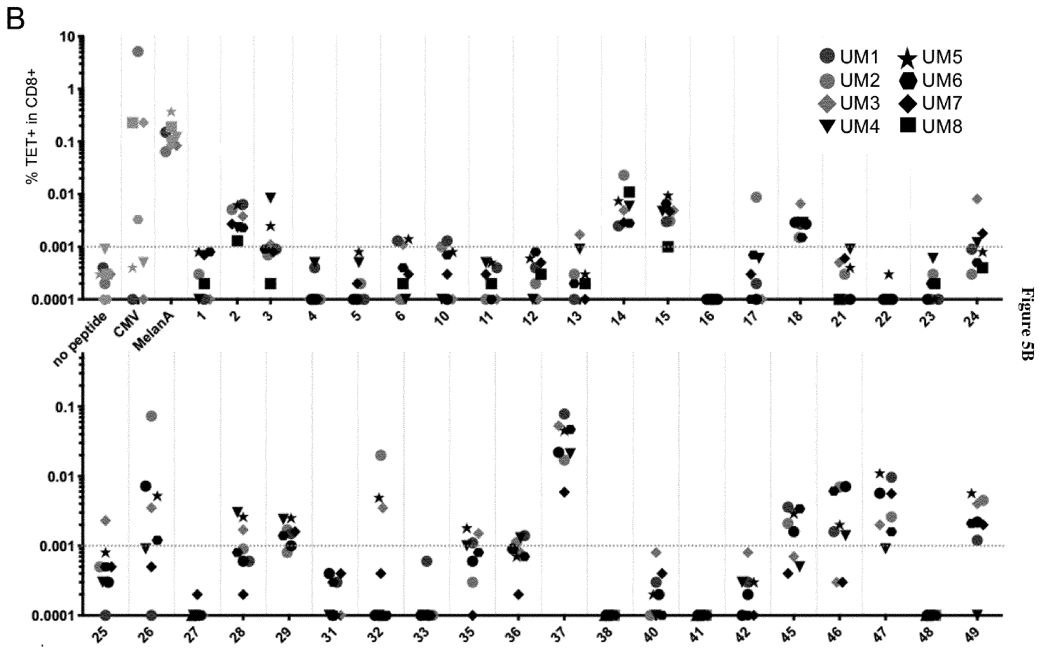

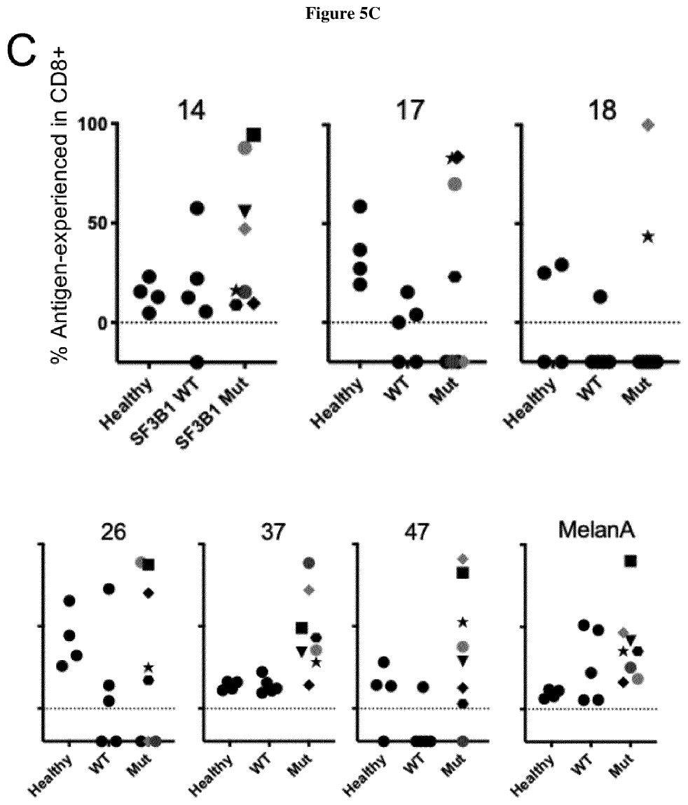

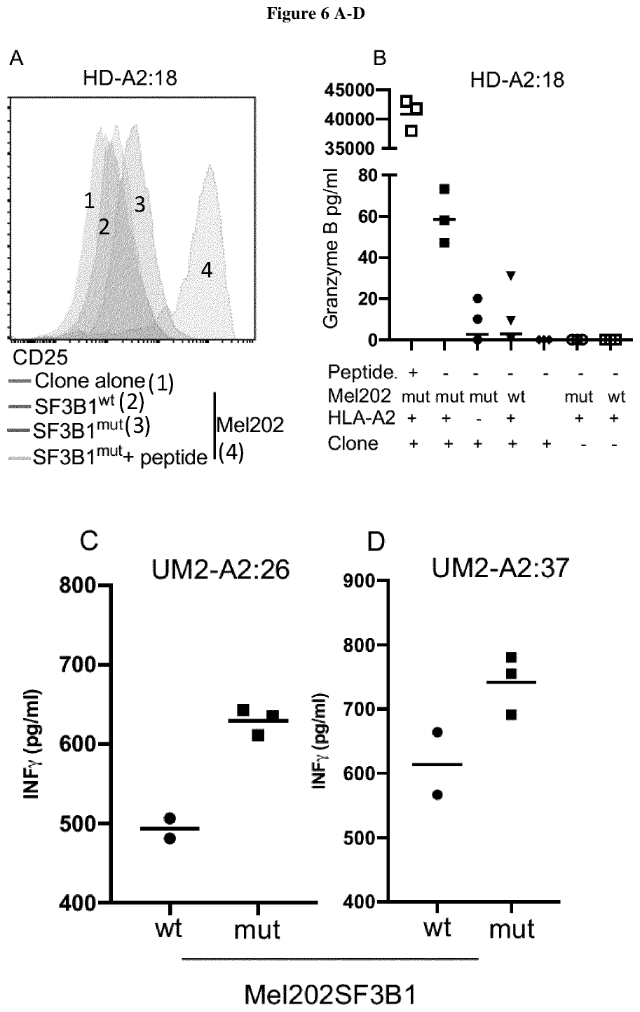

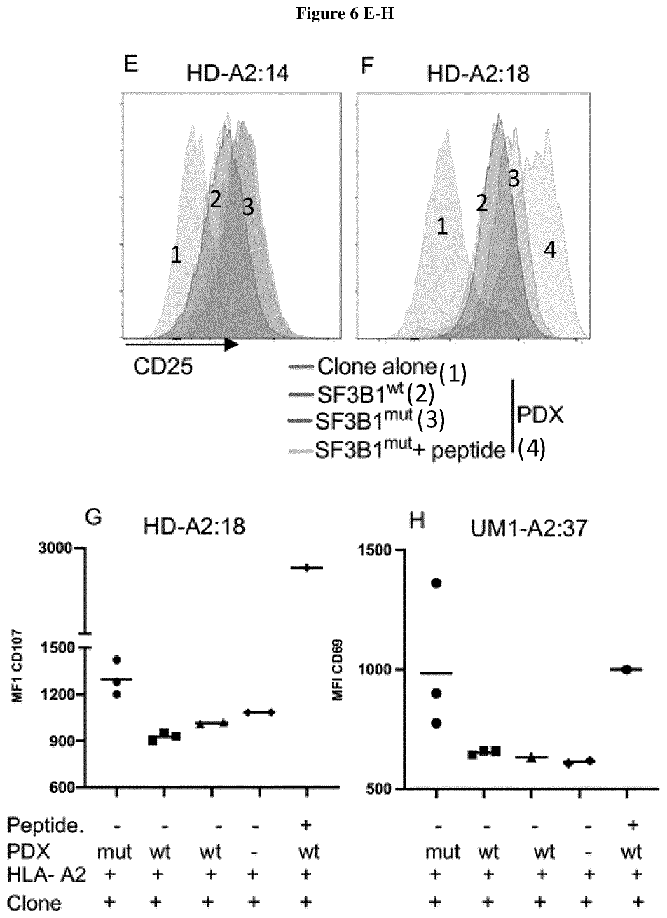

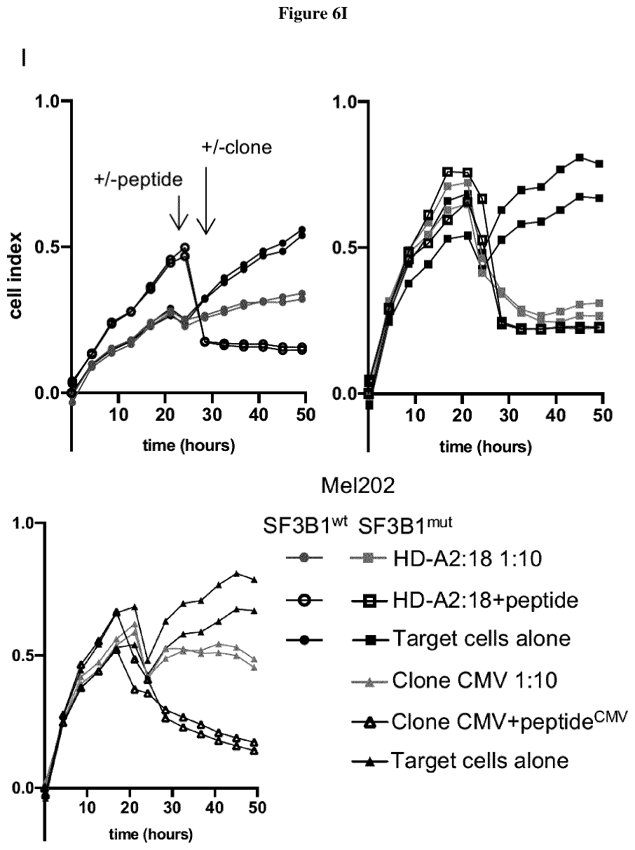

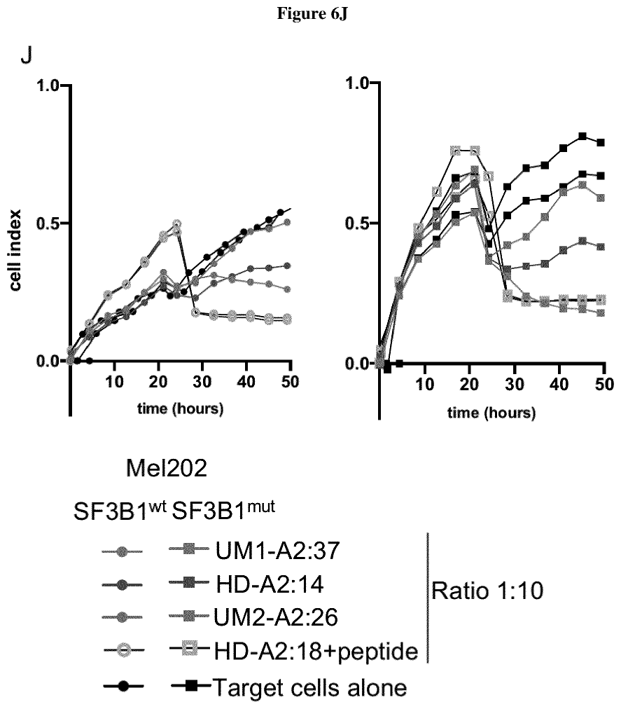

FIG. 6: SF3B1^{mut} tumor cells are specifically recognized and killed by SF3B1^{mut}-related neo-epitope specific T cells. CD8+ T cell clones were co-cultured with Mel202 derived cell lines (A, B, C, D, I, J) or PDX cultures (E, F, G, H). Clone activation upon cell line co-culture was measured by upregulation of activation markers CD25 (A, E, F) and CD69 (H), degranulation marker CD107a (G) expressed as mean fluorescence intensity (MFI), GzmB and INF-γ secretion in supernatants (B, C, D) and specific killing of the target cell lines (I, J). (A, B) Clone HD-A2:18 specific for peptide A2:18 was co-cultured with Mel202-derived cell lines. After 18 hours, clone cells were stained for CD25 expression (A) and the supernatants recovered to measure GzmB content (B). The indicated cell line was incubated beforehand with A2:18 peptide as positive control of clone activation. As negative control, the clone was incubated alone or with HLA-A2⁻Mel202 cells. (C, D) Same strategy than (B) using clones specific for peptides A2:26 (C) and A2:37 (D). (E, F) Clones specific for peptides A2:14 (E), A2:18 (F, G) or A2:37 (H) were co-cultured for 18 hours with 3 HLA-A2+PDX derived from UM patients mutated or not for SF3B1 (FIG. 1B). (I) The ability of SF3BP′″specific T cell clones to detect and kill cells expressing or not their cognate antigen was studied through inhibition of Mel202 growth. The cell lines derived from Mel202 were cultured for 24 hours before clone addition. Clone HD-A2:18 killed only the Mel1202 SF3B1^{wt} cells after A2:18 peptide addition (left panel) while it efficiently killed Mel202 SF3B1^{mut} cell line (middle panel). A clone specific for CMV peptide killed only Mel202 SF3B1^{mut} cells pre-incubated with the CMV peptide (right panel). (J) Clones with other SF3B1^{mut} related specificities (A2:37, A2:14, A:26) did not kill Mel202 SF3B1^{wt} cells (left panel) but killed Mel202 SF3B1^{mut} cells (right panel).

### EXAMPLES

1—Genetic Alterations of SUGP1 Mimic Mutant-SF3B1 Splice Pattern in Lung Adenocarcinoma and Other Cancers

Genes involved in 3 ‘-splice site recognition during mRNA splicing and maturation constitute an emerging class of oncogenes’. SF3B1 is the most frequently mutated splicing factor in cancer and its mutants corrupt branchpoint recognition leading to usage of cryptic 3′-splice sites and subsequent aberrant junctions²⁻⁵. For a comprehensive determination of alterations leading to this splicing pattern, The inventors performed a pan-TCGA splice junction analysis. While cryptic 3′-splice usage was strongly associated with SF3B1 mutations, they also detected 10 SF3B1 wild-type tumors (including 5 lung adenocarcinomas). Genomic profile analysis of these tumors identified somatic mutations combined with loss-of-heterozygosity in the splicing factor SUGP1 in 5 of these cases. Modeling of SUGP1 loss and mutations in cell lines showed that both alterations induced mutant-SF3B1-like aberrant splicing. This study provides the first evidence of the involvement of genetic alterations of SUGP1 as an SF3B1 genocopy in lung adenocarcinoma and other cancers.

During splicing, SF3B1 mediates U2 snRNP recruitment to the branchpoint (BP) by interacting with the intronic pre-mRNA⁶. Cancer-associated SF3B1 Major Hot Spots (SF3B1MHS) are change-of-function mutations targeting codons R625, K666 and K700. SF3B1MHS lead to recognition of an alternative branchpoint (BP′) upstream of the canonical BP, consequent cryptic 3′ splice site (3′ss) usage and an aberrant junction in a subset of mRNA defined by sequence requirements.

For a comprehensive view of pathogenic mutations inducing usage of cryptic 3′ss as observed in a SF3B1-mutant context, the inventors first used a Sequence Bloom Tree (SBT)^{8,9} constructed from RNA-seq data for a total of 11,350 different samples and 33 tumor types from TCGA. They tested occurrence of 1443 aberrant junctions reported in two complementary analyses^{4,5} as consequences of SF3B1MHS. The SBT score, representing the number of these junctions found at least once in raw RNA-seq data (fastq) is a fast and highly sensitive approach for such pre-defined patterns. After adjustment for RNA-seq coverage, the 112 top SBT-score cases were selected following the cutoff determined by the lowest SBT score of a validated SF3B1p.A633V case (table 2, FIG. 1a).

TABLE 2
Tumor cases harbouring the SF3B1-like
splice pattern with no SF3B1 mutation

| Tumor | Sample | Alteration |
|---|---|---|
| LUAD | TCGA-55-7576 | SUGP1_LOH |
| MESO | TCGA-3H-AB3K | SUGP1_p.R625T_LOH |
| LUAD | TCGA-05-4432 | SUGP1_p.L515P_LOH |
| LIHC | TCGA-ZP-A9CV | SUGP1_LOH |
| LUAD | TCGA-78-7542 | SUGP1_p.G519V_LOH |
| LUAD | TCGA-NJ-A4YQ | SUGP1_p.P636L_LOH |
| LUAD | TCGA-91-6829 | SUGP1_p.G26*_LOH |
| LAML | TCGA_AB_2882 | ND (U2AF1_S34Y) |
| UVM | TCGA_WC_A87W | ND |
| SKCM | TCGA_EE_A2A1 | ND |

LUAD: Lung Adenocarcinoma
LIHC: Liver Hepatocellular Carcinoma
MESO: Mesothelioma
LAML: Acute Myeloid Leukemia
*Stop mutation
ND: causal alteration not determined

These high SBT-score cases were thoroughly verified for SF3B1 mutations and further characterized for aberrant splice junctions, together with additional cases including those with mutations in other splice factors as well as control tumor cases without splice factor alteration (449 total cases). Based on the direct analysis of cryptic 3′ splice site usage obtained from RNA-seq data (STAR 2.0.5) in this series, the exhaustive list of the TCGA cases exhibiting SF3B1-like splice aberration was obtained. Principal component analysis of 3′ss usage showed the main source of variation to be SF3B1 mutations. The aberrant splice pattern was validated in 87 cases, including 77 SF3B1-mutated cases (51 SF3B1MHS) (FIG. 1bMethods). Ten tumors showing high levels of the 3′ss pattern but not mutated in SF3B1 (hereafter named SF3B1-like) were detected, including lung adenocarcinomas (LUAD, 5 cases), hepatocellular carcinoma (LIHC, 1 case), mesothelioma (MESO, 1 case), acute myeloid leukemia (LAML, 1 case), skin melanoma (SKCM, 1 case) and uveal melanoma (UVM, 1 case). Mutational analysis of RNA processing genes (GO:0006396) of the 10 SF3B1—like cases revealed mutations in SUGP1 (also known as Splicing Factor 4 or SF4) as the only common event for 5 cases: 4 missense (p.L515P, p.G519V, p.R625T, p.P636L) and 1 stop gain (p.G26*) mutations. Interestingly, these mutations do not target any known interaction domain of SUGP1, including its G-patch domain10. Further analyses using SNP-arrays revealed Loss Of Heterozygosity (LOH) of the SUGP1 locus in all 5 cases and Variant Allele Frequency (VAF) in RNA-seq data was consistent with loss of the wild-type allele. Of note, the p.G26* stop-gain mutation is located at the very beginning of the gene, contrasting with its high 73 level of expression in this sample). This suggests that an alternative initiation of translation bypasses the expected nonsense mediated mRNA decay (NMD). Given that only 8 cases out of the entire TCGA series carried SUGP1 variants with RNA-seq VAF>0.3 and LOH, association between SUGP1 variant+LOH (SUGP/LOH/mut) and SF3B1-like phenotype is highly significant (p<10-8, Fisher's exact test adjusted for multiple testing).

The inventors then further mined the 5 SF3B1-like cases associated with neither SF3B1 nor SUGP1 mutation. Normalized SUGP1 expression levels in 2 cases (1 LUAD and 1 LIHC) were the lowest by orders of magnitude in the corresponding cohorts. Interestingly, they also observed LOH in the SUGP1 locus for these two cases. The remaining three cases (1 each LAML, SKCM and UVM) associated with the SF3B1-like splice pattern were not found altered for SUGP1. Of note, while the LAML case harbored a U2AF/S34Y hotspot mutation, this is not likely to be causal of the SF3B1-like splice pattern as 20 other U2AF1S34Y/F cases showed no evidence of such pattern and U2AF1 mutations are known to drive a different splicing pattern^{1,11}. Taking into account that 5 out of the 10 cases were LUAD, including 4 SUGP1LOH/Mut and 1 case with the lowest SUGP1 expression (SUGP1 Low), they performed a splice junction analysis in the LUAD cohort, comparing 4 SUGP/LOH/Mut, 1 SUGP1 Low, 6 SF3B1MHS and 98 LUAD cases with no splice gene mutation (See Methods). This showed coherent changes in SUGP/LOH/Mut and

SF3B1MHS splice profiles, with the similar profile of aberrant junctions showing increased usage cryptic 3′ ss located 10-25 nts upstream of the canonical 3′ ss (FIG. 1c-d). They then directly assessed the impact of SUGP1 alterations, including LOH and mutations, on splicing. Using HEK293T cells (wild-type for both SUGP1 and SF3B1), they performed siRNA-mediated SUGP1 knockdown and overexpression of the SUGP1 L515P, R625T and P636L mutants. As a readout, they assessed the SF3B1 index, which is the ratio of aberrant to canonical junction expression, in three SF3B/MHS—sensitive junctions: DPH5, DLST and ARMC9, as previously reported4. The knockdown of SUGP1 using 4 different siRNAs consistently and significantly induced the SF3B/MHS-aberrant pattern (p<0.05 to <0.0005, depending on the siRNA and junctions; FIG. 2a). Transiently overexpressed SUGP1 mutants induced either significant but modest effects on splicing (L515P and P636L) or no significant effect (R625T) (FIG. 2b), arguing against strong dominant-negative properties of these mutants. Interestingly, RNA-seq analysis of HEK293T cells transiently depleted for SUGP1 showed aberrant splice events highly correlated with those observed in cells overexpressing SF3B1K700E (Pearson's correlation; r2=0.75, p-value=4 10-42; FIG. 2c). In an SF3B1 mutant context, the U2 complex has a preferential recognition for the cryptic branchpoint BP′. To determine whether SUGP1 loss affects U2 recognition of the BP in a similar manner, they performed a splice-reporter assay with SF3B1MHS-sensitive junctions (ENOSF1 and TMEM14C) containing adenine mutants inactivating either the canonical or cryptic BPs. Their results showed that mutants disrupting the BP′ abolish the splice aberration induced by siRNA-mediated SUGP1 knockdown (FIG. 2d). This finding demonstrates that SUGP1 is critical for correct recognition of the BP by the U2 complex, and that its loss phenocopies SF3B1MHS. To recapitulate the homozygous state of SUGP1 mutants found in tumors, we generated a haploid cell model harboring the SUGP/P636L mutation (HAP1SUGP1-P636L) by CRISPR/cas9 editing. RNA-seq of HAP1SUGP1-P636L compared with the parental HAP1 cell line showed similar splicing aberrations as observed in tumors carrying SUGP1 and SF3B1 mutations (FIG. 3a). HAP1SUGP1-P636L cells displayed splice aberrations consistently with the SF3B/MHS-splice pattern, and mainly characterized by the usage of cryptic 3′ss at 10-25 nts upstream the canonical 3′ss (FIG. 3b-c). The assessment of DPH5 aberrant junction expression confirmed the induction of the SF3B/MHS—splice pattern in HAP1SUGP1-P636L as compared to HAP1(FIG. 3d). Strikingly, siRNA-knockdown of the SUGP/P636L further increased the aberrant splice index, implying that SUGP1 mutations lead to a partial loss of function (hypomorphic mutations) accentuated by the mutant knockdown (FIG. 3d). Splicing is a step-wise process, and assembled splicing complexes have been reported to be inactive unless SUGP1 (SF4) is added¹². They evaluated three potential partners mediating interaction with either SUGP1 or SF3B1: (i) SUGP1 contains an SURP domain that binds to SF1, which initially binds to the branchpoint and recruits the U2 snRNP to the spliceosome13; (ii) SUGP2 is a paralog of SUGP1 harboring similar SURP and G-patch domains; (iii) SPF45 (RBM17) has been reported to be involved in recognition and activation of the cryptic 3′ ss with the help of SF3B1 and SF114. Additionally, SPF45 binds SUGP1, which makes it a potential mediator of the of SUGP1-SF3B1 interaction15. They performed siRNA-mediated knockdown of the three potential splice partners: SF1, SUGP2 and SFP45 and none of these knockdowns induced the SF3B1-like splice pattern, implying an independent mechanism where SF3B1 and SUGP1 share parallel functions. Very recently, the SUGP1-SF3B1 biological pathway was largely elucidated by Zhang and Coll¹⁰, revealing differential and direct interaction between SUGP1 and either the wild-type or the K700E mutant SF3B1. They extend these results here by further demonstrating the direct involvement of SUGP1 mutations in cancer-associated splicing defects, and providing the first evidence of its implication as a recurrent actor in lung adenocarcinoma. Its recurrent genetic alterations in cancer strongly suggest a role in oncogenesis. However, most missense mutations identified in our study are not predicted to have a strong deleterious effect and do not target the major interaction domain found by Zhang et al, arguing for an essential function of SUGP1. Therefore, SUGP1 alterations may be stringently selected in cancer for a subtle reduced activity compatible for survival and required for oncogenesis.
1. Dvinge, H., Kim, E., Abdel-Wahab, O. & Bradley, R. K. RNA splicing factors as oncoproteins and tumour suppressors. Nat Rev Cancer 16, 413-30 (2016).
2. Papaemmanuil, E. et al. Somatic SF3B1 mutation in myelodysplasia with ring sideroblasts. N Engl J Med 365, 1384-95 (2011).
3. Yoshida, K. et al. Frequent pathway mutations of splicing machinery in myelodysplasia. Nature 478, 64-9 (2011).
4. Alsafadi, S. et al. Cancer-associated SF3B1 mutations affect alternative splicing by promoting alternative branchpoint usage. Nat Commun 7, 10615 (2016).
5. Darman, R. B. et al. Cancer-Associated SF3B1 Hotspot Mutations Induce Cryptic 3′ Splice Site Selection through Use of a Different Branch Point. Cell Rep 13, 1033-45 (2015).
6. Gozani, O., Potashkin, J. & Reed, R. A potential role for U2AF-SAP 155 interactions in recruiting U2 snRNP to the branch site. Mol Cell Biol 18, 4752-60 (1998).
7. DeBoever, C. et al. Transcriptome sequencing reveals potential mechanism of cryptic 3′ splice site selection in SF3B1-mutated cancers. PLoS Comput Biol 11, e1004105 (2015).
8. Lau, J. W. et al. The Cancer Genomics Cloud: Collaborative, Reproducible, and Democratized-A New Paradigm in Large-Scale Computational Research. Cancer Res 77, e3-e6 (2017).
9. Dehghannasiri, R. et al. Improved detection of gene fusions by applying statistical methods reveals oncogenic RNA cancer drivers. Proc Natl Acad Sci USA 116, 15524-15533 (2019).
10. Zhang, J. et al. Disease-Causing Mutations in SF3B1 Alter Splicing by Disrupting Interaction with SUGP1. Mol Cell (2019).
11. Ilagan, J. O. et al. U2AF1 mutations alter splice site recognition in hematological malignancies. Genome Res 25, 14-26 (2015).
12. Utans, U. & Kramer, A. Splicing factor SF4 is dispensable for the assembly of a functional splicing complex and participates in the subsequent steps of the splicing reaction. EMBO J 9, 4119-26. (1990).
13. Crisci, A. et al. Mammalian splicing factor SF1 interacts with SURP domains of U2 snRNP-associated proteins. Nucleic Acids Research, gkv952 (2015).
14. Corsini, L. et al. U2AF-homology motif interactions are required for alternative splicing regulation by SPF45. Nature Structural & Molecular Biology 14, 620-629 (2007).
15. Hegele, A. et al. Dynamic Protein-Protein Interaction Wiring of the Human Spliceosome. Molecular Cell 45, 567-580 (2012).

2—Splicing Patterns in SF3B1 Mutated Uveal Melanoma Generate Shared Immunogenic Tumor-Specific Neo-Epitopes

### SUMMARY

Disruption of splicing patterns due to mutations of splicing factors in tumors have been proposed for several years as a source of tumor neo-epitopes, which would be both public (shared between patients) and tumor-specific (not expressed in normal tissues). In this work, we show that mutations of the splicing factor SF3B1 in uveal melanoma (UM) generate immunogenic neo-epitopes. Memory CD8 T cells specific for these neo-epitopes are only found in the 20% of UM patients whose tumor is mutated for SF3B1. Single cell analyses of neo-epitope specific T cells from the blood identified large clonal T-cell expansions with various and distinct effector transcription patterns. Some of these clones were found in the corresponding tumor. Clones of CD8 T cells specific for the neo-antigens specifically recognized and killed SF3B1-mutated tumor cells supporting the use of these germline-encoded neoantigens related to SF3B1 mutations as therapeutic targets.

Methods

Human Samples

Blood and leukaphereses from healthy donors (HD) were provided by the Establishment Français du Sang. Leukaphereses from patients with metastatic uveal melanoma (UM) were obtained from peptide vaccine trials CP-99-03 and IC-2004-01 before treatment. We selected the patients that had more than 30 frozen vials of PBL and for whom liver metastasis RNA was available. Cells were stored in liquid nitrogen until the time of analysis. RNA from the confirmatory diagnostic biopsy of the UM liver metastasis performed at inclusion was used for SF3B1 mutation confirmation and TCR sequencing. SF3B1 was sequenced as previously described (10). In the inventor's institution, all patients were informed that pathological specimens might be used for research purposes.

RNA-Sequencing, Analysis and Neoepitope Prediction

RNA was isolated from fresh tumor samples using a CsCl cushion as described (22) then quantified with Qubit RNA HS assay kit (Thermo Fisher Scientific). RNA-seq libraries were constructed using the TruSeq Stranded mRNA Sample Preparation Kit (Illumina) and sequenced on an Illumina HiSeq 2500 platform using 100-bp paired-end sequencing. An average depth of global sequence coverage of 111 million and a median coverage of 75 million was attained. Differential junctions using alternative acceptors were identified as previously described (10) comparing the 8 SF3B1′ tumors with 5 SF3B1^{wt} tumors. Sequences of aberrant and corresponding normal transcripts were extracted using ANNOVAR and ENSEMBL database (23) NetMHCpan v4, an artificial neural network-based algorithm was used to predict MHC class I affinity for splicing anomalies derived peptides (12). Only nonamer peptides with strong affinity for HLA-A2:01 (rank<0.5% compared to a set of 400,000 random peptides) were retained for this study.

MHC/Peptide Complex Generation:

Recombinant HLA-A*02:01 molecules (13) were purchased from immunAware (Copenhagen, Denmark) as easYmers® (catalog #1002-1). All peptides were synthesized at <95% purity (Synpeptide) and tested for HLA-A2 monomer avidity following immunAware bead-based recommended assay and ELISA (24). Briefly, for each tetramer, MHC/peptide complex at 100 μM were combined 1H at room temperature with fluorescent streptavidin (Biolegend) or oligo-tagged streptavidin (Biolegend) for single cell experiment. Tetramers were stored at 4° C. for maximum 3 months.

Facs Analysis and Abs:

PBMC were thawed in CO₂-independent medium (GIBCO) and incubated during 30 minutes in culture medium containing 50 nM dasatinib (25) to improve tetramer staining. CD8⁺ T cells were enriched using human CD8 T cell enrichment kit (Stemcell) according to manufacturer instructions. Dead cells were stained with live/dead aqua (Invitrogen). For tetramer staining, tetramers for each specificity were labelled separately with 2 different fluorochromes in order to combine 10 different tetramer/peptide complexes in the same experiment and to decrease the noise related to non-specific binding (26). Briefly, cells were incubated for 20 min with each tetramer complex in brilliant stain buffer (BD) then cells were stained for 20 min with indicated antibodies, (CD3-BUV737, CD8-BUV395 (BD), CCR7-BV421 (Biolegend), CD45-RA FITC (Miltenyi Biotec), CD25 (BD), CD8 FITC, CD3 Alexa fluor 700). Cells were then washed and analyzed in a LSR Fortessa cytometer (BD)

T Cell Clone Generation and Cells Culture:

After tetramer staining, double tetramer positive CD8⁺ single cells were FAC sorted into 96 wellcntaining 1:1 AIM-V/RPMI medium supplemented with 5% human serum, 100 U/mL penicillin, 100 μg/mL streptomycin in the presence of 2×10⁵ irradiated (50 Gy) allogenic feeder cells. Cells were stimulated with human IL-2 (Novartis) (3000 UI/ml) and anti-CD3 (OKT3) (30 ng/ml). Starting on day 5, half of media was replaced with a 1:1 mixture of AIM-V/RPMI containing IL-2 (3000 IU/ml) every three days. When lymphocyte growth was evident, clones were transferred into T25 flasks. The clones were re-stimulated every 3 weeks using the same media containing IL-2, OKT3 and irradiated feeders. After each cycle of clone amplification, each clone was tested for tetramer binding by cytometry and their capacity to respond to peptide stimulation using IFN-γ and GrzB secretion (BD) and/or intracellular IFN-γ staining (eBioscience). cDNA from each clone was amplified by PCR using primers for TRAY, TRBV and constant regions (27), the PCR products sequenced and the resulting sequences analyzed using IMGT/V-QUEST (28).

Clone activation, cytokine release and killing assay: T cells clones were cocultured at 1:1 ratio with indicated cell line pulsed or not with 15 μM to 0.3 pM of peptide in AIM-V/RPMI medium for 18 hours at 37° C. Activation was measured by CD69, CD25, CD107a staining while cytokine secretion was analyzed in supernatant using cytometric beads array kits (BD) according to manufacturer's instruction. Killing assay was performed by culturing SF3B1′ or SF3B1′ Mel202 cell line.

Cell Lines

A Degron-KI system was used to generate isogenic cell lines from Mel202, a uveal melanoma cell line mutated for SF3B1 (c.R625G) as described in (15). Shortly, a Degron sequence was inserted by CRISPR/cas9 5′ to the start codon of the mutated SF3B1 allele. An expression vector for HLA-A2 kindly provided by 0. Schwartz (29) was stably integrated in both wild-type and edited Mel202 cell lines and validated by FACS analysis for HLA-A2 membrane expression using BB7.2-FITC antibody (BD).

Single Cell Experiments:

Thawed PBMC from patient UM1 were stained with PE and APC tetramers loaded with peptide 37, then tetramer positive cells were positively enriched using anti-APC and anti-PE microbeads (Miltenyi Biotech), stained with CD3 A700 and CD8 FITC and finally with DAPI. The positive fraction was sorted in a FACS ARIA (BD). To combine 5 tetramer positive populations from 2 donors (14, 17, 26 and 37 for UM2 and 18 for UM3) the tetramers were prepared using 5 different TotalSeq-PE streptavidins (BioLegend) and 1 classical fluorochrome-streptavidin (APC, PE-CF594, PE-Cy5, PE-Cy7), PE-CF594 was used for UM3 who had only one population sorted. The PBMC were stained with the 4 pairs of tetramers for patient UM2 and 1 pair of tetramers for patient UM3, enriched with anti PE-microbeads (Milteny Biotech), stained with CD3 A700 and CD8 FITC and DAPI and sorted separately. The cells were then counted, mixed and loaded onto a Chromium controller using Chromium next GEM Single Cell V(D)J reagent kit with feature barcoding technology according manufacturer's instructions.

Single-Cell RNA-Seq Processing

Single-cell expression was analyzed using the Cell Ranger single-cell Software Suite (v3.0.2, 10× Genomics) (30) to perform quality control, sample de-multiplexing, barcode processing, and single-cell 5′ gene counting. Sequencing reads were aligned to the GRCh38 human reference genome. Further analysis was performed in R (v3.5.1) using the Seurat package (v3.1.1) (31). Cells were then filtered out when expressing less than 500 genes for UM1 and 1000 genes for UM2/UM3 since this sample was of lower quality. Cells were also filtered out when expressing more than 10% mitochondrial genes, indicative of potential cell death or stress. Samples were then filtered for contaminating cells using classical markers. Notably, CD19 was used to remove B cells, MAFB was used to remove myeloid cells and CD3D/E/G and CD8A/B were used as positive controls. Altogether, 3441 cells were kept for UM1 and 3231 were kept for UM2 and UM3. For each sample, the gene-cell-barcode matrix of the samples was then normalized to a total of 1×10⁴ molecules. TotalSeq values were normalized according to the CLR method implemented in Seurat. The top 2000 variable features were identified using the “vst” method from Seurat. For UM2/UM3 samples, the fraction of doublets could be removed leveraging the TotalSeq information. Since the TotalSeq features were bimodal, we first binarized the TotalSeq features. The expression threshold was defined as 1 for UM2-A2:26 and 1.2 for the rest of the specificities. Cells were then labelled cells as doublets if they were expressing more than 1 TotalSeq above the expression threshold. 490 cells out of the 3213 (15%) were removed after removing TotalSeq doublets.

Dimension Reduction and Unsupervised Clustering

Top 30 Principal Components were computed and UMAP was performed using the top 30 PCs of the normalized matrix. Clusters were identified using the FindNeighbors and FindClusters function in Seurat with a resolution parameter of 0.4 for UM1 and 0.35 for UM2/UM3 and using the first 30 principal components. To choose the optimal number of clusters and prevent overclustering, clustree analysis was performed using the clustree package (32). Unique cluster-specific genes were identified by running the Seurat FindAllMarkers function using Wilcoxon test.

Analysis of Aberrant Peptides by Mass Spectrometry

Endogenous NET1 was immunoprecipitated from protein extracts using of antibody targeting the N-terminus part of NET1 (sc-271941; 2 μg per 200 μg of cell extract), as described (Kweh F, Zheng M, Kurenova E, Wallace M, Golubovskaya V, Cance W G. Neurofibromin physically interacts with the N-terminal domain of focal adhesion kinase. Mol Carcinog. 2009; 48:1005-17.). Peptidic samples were analyzed using an Orbitrap Exploris 480 mass spectrometer (Thermo Scientific) coupled to a RSLCnano system (Ultimate 3000, Thermo Scientific). To identify the endogenous aberrantly spliced NET1, cell lines and PDXs were lysed in Lysis buffer (50 mM Tris, 150 mM NaCl, 1% Triton, 0.5% NaDOC, 0.1% SDS, 5 mM EDTA, 10% glycerol supplemented with protease inhibitors (Roche) and phosphatase inhibitors) for 30 min on ice. Insoluble material was pelleted by centrifugation (16,000 g, 15 min at 4° C.). Lysates were then pre-cleared by incubation with 10 μL of nonspecific mouse IgG (Santa Cruz, sc-2025) plus 20 μL of packed and pre-washed Dynabeads coupled with protein G (10003D, Life Technologies) for 30 minutes at 4° C. The non-specific antibodies were then removed using magnetic rack and 250 μL of pre-cleared supernatant was incubated with 20 μL of anti-NET1 antibodies (sc-271941, Santa Cruz) overnight under gentle rotation. The immunocomplexes were then incubated with 20 μL of packed protein G coupled Dynabeads (10003D, Life Technologie) for 20 minutes at RT under gentle rotation.

Immunocomplexes bound to the beads were pelleted using magnetic rack and wash once with washing buffer (300 mM NaCl, 150 mM KOAc, 50 mM Tris, 2 mM MgCl₂, phosphatase and protease inhibitors) containing 1% NP-40-substitute and 3 times with washing buffer containing 0.1% NP40-substitute. The immunocomplexes were finally washed 3 times with 500 μL of 25 mM Ammonium bicarbonate.

Finally, beads were resuspended in 100 μl of 25 mM NH₄HCO₃ and digested by adding 0.4 μg of trypsine/LysC (Promega) for 1 hour at 37° C. The resulting peptide mixtures were then loaded onto homemade C18 StageTips packed with AttractSPE™ Disks Bio C18 (Affinisep™ SPE-Disks-Bio-C18-100.47.20) for desalting. Peptides were eluted using 40/60 MeCN/H2O+0.1% formic acid, vacuum concentrated to dryness and reconstituted in injection buffer (2% MeCN/0.3% TFA).

Liquid chromatography-mass spectrometry (LC-MS/MS) analysis. LC was performed with an RSLCnano system (Ultimate 3000, Thermo Scientific) coupled online to a Orbitrap Exploris 480 mass spectrometer (MS).

Peptides were trapped on a C18 column (75 μm inner diameter×2 cm; nanoViper Acclaim PepMap™ 100, Thermo Scientific) with buffer A (2/98 MeCN/H₂O in 0.1% formic acid) at a flow rate of 3.0 μL/min over 4 min. Separation was performed on a 50 cm×75 μm C18 column (nanoViper Acclaim PepMap™ RSLC, 2 μm, 100 Å, Thermo Scientific) regulated to a temperature of 40° C. with a linear gradient of 3% to 29% buffer B (100% MeCN in 0.1% formic acid) at a flow rate of 300 nL/min over 91 min. MS full scans were performed in the ultrahigh-field Orbitrap mass analyzer in ranges m/z 375-1500 with a resolution of 120 000 at m/z 200. In the data-dependent acquisition (DDA) mode, top 15 intense ions were subjected to Orbitrap for further fragmentation via high energy collision dissociation (HCD) activation and a resolution of 15 000 with the auto gain control (AGC) target set to 100%. We selected ions with charge state from 2+ to 6+ for screening. Precursor ions were isolated with an isolation width of 1.6 m/z unite, normalized collision energy (NCE) was set to 30% and the dynamic exclusion to 40 s. In parallel reaction monitoring (PRM) mode acquisition list (Table 1.) was generated from the peptides obtained from the synthetic aberrant NET1 peptides (TALLPGLPAANPSPR and LFPISPETLHFPVSR, ordered from Genscript at purity >75%) based on the DDA results (data not shown).

TABLE 1

| Peptide Modified | Mass | | |
|---|---|---|---|
| Sequence Full Names | [m/z] | Charge | Extracted fragments |
| LFPISPETLHFPVSR | 580.6542 | 3 | y4, y5, y6, y10, y10++, |
| | | | y11++ |
| TALLPGLPAANPSPR | 737.9225 | 2 | b3, y8, y8++, y10, y11, |
| | | | y11++ |

PRM data analysis: all raw files were processed using Skyline (version 13.1.1.193) MacCoss Lab Software, Seattle, Wash.; (https://skyline.ms/project/home/software/Skyline/begin.view) for the generation of the extracted-ion chromatograms and peak integration. To robustly identify peptides in the skyline platform, a mass accuracy of withing 5 ppm was imposed for fragment ions. The targeted peptides were manually checked to ensure that the transitions for multiple fragment ions exhibit the same elution time in the pre-selected retention time window of the synthetic peptide. The data were then processed so that the distribution of relative intensities of multiple transitions associated with the same precursor ion must be correlated with the theoretical distribution in the MS/MS spectral library entry. The assessment of MS/MS matching was performed by Skyline and Proteome Discoverer (version 2.4). It is worth noting that the same retention time and dot product (dotp) values (1) of at least 0.9 were found for all PRM transitions, thereby providing accurate peptide identification.

For protein identification, the data were searched against the Homo sapiens (UP000005640) UniProt database with the two aberrant sequences of NET1 using SequestHT Proteome Discoverer (version 2.4). Enzyme specificity was set to trypsin and a maximum of two-missed cleavage sites was allowed. Oxidized methionine, Met-loss, Met-loss-Acetyl and N-terminal acetylation were set as variable modifications. Maximum allowed mass deviation was set to 10 ppm for monoisotopic precursor ions and 0.02 Da for MS/MS peaks. The resulting files were further processed using myProMS v3.9.2. FDR calculation used Percolator and was set to 1% at the peptide level for the whole study.

The mass spectrometry proteomics data have been deposited to the ProteomeXchange Consortium (http://proteomecentral.proteomexchange.org) via the PRIDE partner repository (3) with the dataset identifier PXD023968 corresponding to the X2185VM, X2186VM, X2188VM, X2189VM datasets.

Single-Cell TCR-Seq Data Processing and Analysis

TCR-seq data for each sample was processed using Cell Ranger software with the command ‘cellranger vdj’ using the human reference genome GRCh38. Because of dropouts, both TCRα and TCRβ are not always sequenced in a given T cell. Thus, as a T cell can express up to two TCRα chains and one TCRβ chain, it is easy to artificially split true T cell clones into two different clonotypes. To the contrary, incompletely sequenced doublets can mistakenly lead to the creation of artefactual clonotypes. Because, the obtained data sets encompassed very large clonal expansions and the drop-out and the number of cells loaded on the chip was high for patient UM2/3, data sets, the inventors manually curated all the recurrent clonotypes to exclude doublets and merge clonotypes. They merged clonotypes using the same TCRα or TCRβ chains. The inventors excluded from downstream analysis all “cells” made of a non-attributed TCRα or TCRβ chains that would be associated with a TCRα or TCRβ chains belonging to a clonal expansion. TotalSeq features were also used to exclude doublets.

TCR-Seq

Reverse transcription of tumor RNA was performed using random hexamers and SuperScript IV according to manufacturer instruction (ThermoFisher). cDNAs were cleaned using Agencourt RNAclean XP kit (Beckman Coulter). A combination of Va and VP specific primers slightly modified from (27) was used in 2 semi-nested PCR steps followed by a barrecoding step. The first PCR reaction was performed separately for alpha and beta TCRs using multiplex Va and VP primer associated with constant TCRα (TRAC) and TCRβ (TRBC) region primers. Each primer was used at 0.2 μM each (95° C. 3 min) and 22 cycles (90° C. 30s, 58° C. 30s, 72° C. 30s). cDNAs were cleaned using Agencourt AMPure XP kit. In the second step, two distinct semi nested PCR multiplex for Va and VP reactions were performed 95° C. 3 min followed by 35 cycles (90° C. 30s, 63° C. 30s, 72° C. 30s). Barcoding and incorporation of the sequencing primers for Paired-end Illumina sequencing was performed with PE1_CS1 forward primer and PE2_barcode_CS2 reverse primer (Fluidigm) at 400 nM using Platinium Taq DNA Polymerase High Fidelity (ThermoFisher). PCR product were sequenced using Miseq V3 PE-300 kit (I lumina)

Results

Activation of the immune system by various immunomodulators can eradicate large tumors as well as the disseminated disease. Tumor destruction is most often related to the recognition by T cells of peptides derived from somatically mutated proteins expressed by cancerous cells and presented by major histocompatibility complex (MHC) molecules. Indeed, the clinical response to anti-checkpoint treatments is loosely correlated with the number of somatic mutations present in the tumor suggesting that the neo-epitope load expressed by the tumor is important to generate an efficient immune response (1). However, most of these neo-epitopes correspond to passenger mutations that are different in each tumor and thus are unique to each patient. In the absence of a spontaneous response that could be therapeutically amplified, inducing an immune response to such epitopes requires personalized vaccines which are costly and logistically complicated to set up (2). For these reasons, public (shared between individuals) epitopes deriving from germline encoded antigens that are aberrantly expressed in tumors with limited expression in normal tissues, such as onco-testis antigens, are often used in vaccine strategies (3). However, many of these antigens are also expressed in the thymus, potentially leading to deletion of the high avidity antigen reactive T cells (4).

An alternative vaccine strategy would be to target neo-epitopes resulting from cancer specific mutations in splicing factors (5) that are mutated in a notable proportion of tumors (6). Indeed, mutations in these factors lead to the presence of aberrant open reading frames specifically in tumor cells. The inclusion of intronic sequences in the mature transcript results in new peptides often with an additional frameshift in the following exon. Since the peptides are germline-encoded, they are present in all tumors bearing the mutated splicing factor. When presented by the patient MHC alleles, the resulting peptides generate neo-epitopes common to all patients bearing a given MHC allele opening the way for generic therapeutic products adapted to common MHC haplotypes. Although proposed for several years (7), the clinical relevance of neo-epitopes resulting from mutated splicing factors in tumors has not been demonstrated in humans so far.

Uveal melanoma (UM) is a rare disease (˜600 cases/year in France) with a dismal prognosis once metastatic, which occurs in more than 30% of cases and for which no therapy is currently available (8). Contrary to skin melanoma, UMs display very few somatic mutations (16±4.0 per exome, (9)) and are accordingly resistant to anti-checkpoint immunomodulation. Twenty percent of the tumors harbor a mutation in the splicing factor 3b subunit 1 (SF3B1) gene generating over 1000 new splice junctions (10, 11). SF3B1 mutations induce an upstream shift of the splice acceptor sites leading to inclusion of intronic sequences in the mRNA. The resulting additional amino-acids and the frameshift that is often associated potentially generate a large number of public neo-epitopes.

In this work, the inventors show that among metastatic UM patients, only those whose tumors harbored a mutated SF3B1 displayed memory CD8⁺ T cells with specificities for SF3B1^{mut}-derived neo-epitopes. The corresponding TCRs could also be found in the tumor. SF3B1^{mut} UM cell lines were recognized and killed by neo-epitope specific T cell clones demonstrating that these neo-epitopes are expressed by tumor cells in a way that can be recognized by CD8⁺ T cells.

Selection of the Patients and Identification of SF3B1^{mut} Induced Epitopes

Thirteen HLA-A2 metastatic UM patients (8 SF3B1^{mut} and 5 SF3B1^{wt}) and 4 healthy donors were studied. It was first verified that the tumors were correctly classified by resequencing SF3B1 in tumors. Based on the previous work of the inventors (10), RNA-seq of the tumors allowed us to measure the proportion of SF3B1^{mut}-modified splice junctions as compared to the SF3B1^{wt} tumors. The pattern of junctions according to SF3B1 status reproduced our previous results (10). They also verified the expression pattern of selected alternative junctions in 3 HLA-A2⁺ patient-derived xenografts (PDX): 1 SF3B1^{mut} and 2 SF3B1^{wt} PDXs (FIG. 4A). it was then identified the SF3B1^{mut}-induced neo-reading frames predicted to generate 9 amino-acid (AA) long peptides able to bind HLA-A2 according to a publicly available bio-informatics pipeline (12) (FIG. 4B). The inventors selected the 0.5% most avid peptides (n=43) (FIG. 4C) and generated the corresponding HLA-A2:peptide complexes using empty monomers (13). They verified their stability and discarded 4 peptides with low HLA-A2 binding, allowing them to make tetramers able to detect specific T cells for 39 HLA-A2 restricted SF3B1^{mut}-related peptides.

The alternative splice junctions may give rise to proteins, which may however be unstable and difficult to detect using biochemical methods. As a proof of concept, the inventors focused on the NET1 protein, in which an alternative junction is predicted to lead to an alternative transcript encoding a polypeptide generating epitope. An immunoprecipitating antibody for a domain upstream of the alternative coding-frame aberrant-splicing event was available (detailed in Material and Methods) and used to immune-precipitate endogenous NET1 from two UM cell lines (MP41 and Mel202; SF3B1wt and SF3B1mut respectively) as well as two PDXs (PDX-MP41 and PDX-MM267; SF3B1 wt and SF3B1mut respectively). After trypsin digestion, the immunoprecipitated samples were analyzed by targeted mass-spectrometry. As shown by their retention time and MS fingerprints comparatively similar to the synthetic peptides, the two predicted peptides corresponding to the aberrant reading frame were readily detected in SF3B1mut cells (Mel202 and PDX-MM267), but absent in the SF3B1wt cells (MP41 and PDX-MP41), whereas the wildtype NET1 peptides were present in all samples. These findings obtained for NET1 demonstrate that aberrantly spliced transcripts in SF3B1mut cells can be translated into detectable aberrant protein products.

Frequency and Phenotype of SF3B1^{mut}-Related HLA-Epitopes Reacting CD8 T Cells.

In order to detect and characterize a potential immune response against the SF3B1^{mut} related neo-epitopes, The inventors stained blood CD8⁺ T cells from UM patients and healthy controls with tetramers labeled with two different fluorochromes to increase specificity, thereby sensitivity. As controls, they used HLA-A2 tetramers loaded with pp65 from cytomegalovirus (CMV) and with Melan-A, a melanocyte differentiation antigen (FIG. 5A). In a CMV⁺ healthy control, a well-defined cluster of HLA-A2-CMV tetramer⁺ (A2:CMV) CD8⁺ T cells was present in the blood and displayed an effector (CD45RA⁺CCR7⁻) or memory (CD45RA⁻CCR7⁺) phenotype while all HLA-A2:Melan-A tetramer⁺ (A2:Melan-A) CD8⁺ T cells were naïve (CD45RA⁺CR7⁺) (FIG. 5A). In UM patients, a large proportion of A2:Melan-A CD8⁺ T cells displayed an effector/memory phenotype (FIG. 5A) as previously reported (14), confirming that the immune system is stimulated by this melanoma differentiation antigen. The inventors then analyzed the frequency and phenotype of CD8⁺ T cells specific for the SF3B1^{mut}-related neo-epitopes. The frequency of CD8⁺ T cells specific for the SF3B1^{mut}-related epitope HLA-A2: peptide 37 (A2:37) was very high in both patients and controls, similarly to A2:Melan-A (FIG. 5A, B). Notably, these A2:37-specific CD8⁺ T cells were naïve in both healthy controls and SF3B1^{wt} UM patients, but >40% of these cells were effector/memory in 7 out of 8 SF3B1^{mut} UM patients (FIG. 5A, C). These results indicate that the SF3B1^{mut}-derived A2:37 epitope has directly or indirectly been seen by the immune system exclusively in SF3B1^{mut} UM patients.

The frequency of CD8⁺ T cells specific for the 39 HLA-A2 restricted SF3B1^{mut} neo-epitopes varied between <0.0001% to 0.3% in the patients with SF3B1^{mut} tumor (FIG. 5B). Notably, patient UM2 harbored increased frequencies of CD8 T cells specific for several epitopes suggesting a coordinated immune response towards SF3B1^{mut}-derived neo-epitopes. Moreover, the proportion of effector or memory cells in CD8⁺ T cells specific for SF3B1^{mut} related neo-epitopes was increased in patients with SF3B1′ tumors (FIG. 5C) in comparison with both healthy and SF3B1^{wt} UM patients (FIG. 5C), suggesting an immune response towards several SF 3B1^{mut}-related neo-epitopes.

Characterization of T Cells Specific for the SF3B1-Induced A2 Restricted Neo-Epitopes in Three Patients.

To characterize the SF3B1^{mut}-induced specific T cells, the inventors isolated A2:37 specific CD8⁺ T cells from the blood of patient UM1 to analyze their transcriptome and TCR repertoire by single cell RNA sequencing (scRNA-seq) coupled to VDJ analysis. After quality control and filtering, 3213 A2:37-specific CD8⁺ T cells could be divided into 7 expression clusters Cluster #1 (n=355 cells) expressed SELL and LEF1, characteristic of naive CD8⁺ T cells and probably corresponds to the 8% naive A2-37 specific T cells found in this patient by flow cytometry (FIG. 5A). Cluster #2 (n=1416) display a cytotoxic (GZMK/H) and a central memory (CCR7, SELL) phenotype_(. Cluster #3 (n=995) included cells expressing ZNF683 (HOBIT) and ITGB1 (CD49a), both associated with tissue residency and expressed XCL1 and XCL2, two chemokines secreted by CD8⁺ T cells to attract dendritic cells. Cluster #4 cells (n=235) shared many features with clusters #2 and #3 but also expressed FOS, JUNB, CD69, NR4A1, NR4A2, TNF, and IFNG, indicating TCR activation. Cluster #5 cells (n=110) expressed CCR4 (implicated in homing to tissues), TNFRSF4 (CD134) implicated in T cell survival and helper function as well as intermediate level of SELL and CCR7, but very low levels of cytotoxic or chemokine molecules, compatible, with a circulating helper-like function. Cluster #6 (n=56) expressed CCR9, CCR6, KLRC1 and CCLS (FIG. 3B). Cluster #7 is very small (n=46) and expresses TCRγ genes and may encompass γδ T cells fished out by the HLA-A2:37 tetramer due to the intrinsic cross reactivity of TCRs. Among the 2780 cells in which at least one TCRα and/or TCRβ chain was retrieved, one clone was strikingly expanded and represented ˜80% (2259) of the cells. Interestingly, this clone represented most if not all of the cells in the neighbor clusters #2-4, suggesting that clonal circulating cells specific for a given epitope may display distinct but related functional and trafficking features. The second and third most abundant clones (50 and 43 cells) represented most of the circulating helper-like cluster #5 cells while the fourth clone (17 cells) was found in the CCR6/9 cluster #6. The TCRs retrieved only once (singletons) encompassed both naïve cluster #1 cells (n=182) and effector memory cells (n=239). Altogether, these results indicate that T cell clones specific for one given tumor neo-epitope display several differentiation patterns associated with the expression of distinct TCRs. Moreover, for the most abundant clonotype #1, cells were either cytotoxic (cluster #2) or tissue resident (cluster #3) with a further portion being activated (cluster #4).

Using FAC sorting of oligo-barcoded tetramers followed by 5′ transcriptome and VDJ single cell 10× technology, the inventors analyzed the transcriptome and TCR repertoire of the blood CD8⁺ T cells specific for A2:18 in patient UM3 and 4 SF3B1^{mut}-related specificities (A2:14, :17, :26 and :37) in patient UM2, three of which were strikingly increased in the blood of this patient (FIG. 4B). After quality control and filtering, 3231 cells could be divided into 7 clusters, whose transcriptome patterns corresponded to various effector/memory clusters and one naïve subset (#1). The CD8⁺ T cells specific for A2:14, :17, :26 displayed transcriptome patterns corresponding to various types of effector or memory subsets with very few naïve cells. In contrast, the A2:37 specificity encompassed about 50% naïve and 50% effectors cells (FIG. 3E) in agreement with the cytometry data (FIG. 5C). Repertoire analysis demonstrated large TCR expansions, making up to 94% of the T cells for a given specificity. As in patient UM1, each clonotype expressed a particular transcriptome pattern. The number of expanded T cell clones was higher for UM2-A2:37, but their size was smaller. For UM2-A2:37, the inventors also observed a large number of non-recurrent TCRs (singletons), 20% of them expressing various effector/memory transcriptome patterns and 80% being naive. Altogether, expanded T cell clones specific for 5 SF3B1^{mut}-related specificities expressing distinct effector/memory transcriptome patterns were found in the blood of the 3 patients indicating previous contact with antigen.

T Cells Specific for SF3B1^{mut} Induced Neo-Epitopes are Also Found in the Tumor.

To determine in patient UM1 whether A2:37 specific T cells were present in the tumor, the inventors amplified all the TCRα and TCRβ chains in UM1 liver metastasis RNA and deep sequenced them (Table 3 below). Eighteen A2:37 specific TCRs found in the blood were also detected in the tumor. Notably, 5 out of 18 (28%) recurrent TCRs in the blood, including the most abundant one #1, were found in the tumor. The 13 TCR singletons found in the tumor belonged to the effector memory clusters. These results indicate that the some of the most expanded T cell clones and singletons from blood are found in the UM liver metastasis. Interestingly, the clonally expanded blood T cells whose TCR was found in the tumor belonged to 6 out of 7 of the transcriptional clusters indicating various differentiation patterns. These results suggest that the effector/memory A2:37 specific T cells found in blood, some of which are highly amplified, may represent an ongoing anti-tumor immune response at the tumor site. In patient UM2, although their clonal size was much smaller than in patient UM1, only TCRs corresponding to A2:26 and A2:37 specificities were found in the tumor. All these TCRs belonged to effector or memory clusters with various transcriptome patterns. Interestingly, in this patient UM2 harboring increased frequency of memory CD8⁺ T cells specific for several SF3B1^{mut}-related neo-epitopes, an unusually large lymphoid infiltrate was observed in the liver metastasis. Thus, some of the expanded clonotypes found in the blood were also observed in the tumor of the two patients harboring increased frequency of SF3B1^{mut} neo-epitope specific CD8⁺ T cells suggesting an active immune response towards SF3B1^{mut}-related neo-epitopes at the tumor site.

TABLE 3

| HLA | | | | | | |
|---|---|---|---|---|---|---|
| re- | | | | | | |
| stricted | | | | | | |
| Peptide | TRAV | TRAJ | CDR3 TCR alpha AA | TRBV | TRBJ | CDR3 TCR beta AA |
| A2:14 | TRAV12-2 | TRAJ26 | CAFDNYGQNFVF | TRBV7-8 | TRBJ2- | CASSPMDRDEQYF |
| | | | | | 7 | |
| A2:14 | TRAV1-1 | TRAJ34 | CAVRSSYNTDKLIF | TRBV4-3 | TRBJ1- | CASSQESVGSNQPQHF |
| | | | | | 5 | |
| A2:14 | TRAV27 | TRAJ24 | CAGGMTTDSWGKL | TRBV7-9 | TRBJ1- | CASSPGTGVTKDGYTF |
| | | | QF | | 2 | |
| A2:14 | TRAV24 | TRAJ37 | CAFDRGSSNAGKLIF | TRBV2 | TRBJ2- | CASEGVHEQFF |
| | | | | | 1 | |
| A2:17 | TRAV35 | TRAJ26 | CAGLPYGQNFVF | TRBV18 | TRBJ1- | CASSPVGWGNTIYF |
| | | | | | 3 | |
| A2:17 | TRAV12-1 | TRAJ28 | CVVNIPLYSGAGSYQ | TRBV19 | TRBJ2- | CASSYKAEPIYNEQFF |
| | | | LTF | | 1 | |
| A2:17 | TRAV29 | TRAJ47 | CAAREYGNKLVF | TRBV7-6 | TRBJ2- | CASSQLGETGELFF |
| | | | | | 2 | |
| A2:17 | TRAV26-1 | TRAJ42 | CIVGGTALENYGGS | TRBV29-1 | TRBJ1- | CGGQGYHTEAFF |
| | | | QGNLIF | | 1 | |
| A2:17 | TRAV14 | TRAJ42 | CAMREGTLRGSQG | | | CASSLEAPGVISGANVLT |
| | | | NLIF | | | F |
| A2:17 | TRAV14 | TRAJ3 | CAMSLYSSASKIIF | TRBV7-9 | TRBJ2- | CASSLDLRQNEQFF |
| | | | | | 1 | |
| A2:17 | TRAV12-3 | TRAJ12 | CAMSGVDSSYKLIF | | | |
| A2:17 | TRAV13-1 | TRAJ21 | CAASRYGNNFNKFY | TRBV29-1 | TRBJ1- | CSVDWYGGLTNTEAFF |
| | | | F | | 1 | |
| A2:17 | TRAV29 | TRAJ49 | CAASAPGNQFYF | TRBV9 | TRBJ2- | CASSAEGSWGQETQYF |
| | | | | | 5 | |
| A2:17 | TRAV29 | TRAJ29 | CAASTSYSGNTPLVF | TRBV14 | TRBJ1- | CASSQGGGGNQPQHF |
| | | | | | 5 | |
| A2:17 | TRAV21 | TRAJ20 | CAVRGSNDYKLSF | TRBV4-3 | TRBJ2- | CASSFLAGGPNEQYF |
| | | | | | 7 | |
| A2:18 | TRAV17 | TRAJ53 | CATDKGSGGSNYKL | TRBV14 | TRBJ2- | CASSTMGDYYEQYF |
| | | | TF | | 7 | |
| A2:18 | TRAV24 | TRAJ20 | CAFWSAYKLSF | | | |
| A2:18 | TRAV25 | TRAJ26 | CAGRDNYGQNFVF | TRBV7-8 | TRBJ1- | CASSPWGAGNQPQHF |
| | | | | | 5 | |
| A2:26 | TRAV8-2 | TRAJ49 | CAVRNTGNQFYF | TRBV15 | TRBJ1- | CAINSGFGSPLHF |
| | | | | | 6 | |
| A2:26 | TRAV17 | TRAJ43 | CATDDDMRF | | | |
| A2:26 | TRAV13 | TRAJ26 | CAAPGNYGQNFVF | TRBV20-1 | TRBJ2- | CSASQIYEQYF |
| | | | | | 7 | |
| A2:26 | TRAV35 | TRAJ30 | CAGIVRDDKIIF | TRBV7-7 | TRBJ2- | CASSFSLQYEQYF |
| | | | | | 7 | |
| A2:26 | TRAV22 | TRAJ30 | CAALGGDKIIF | TRBV7-6 | TRBJ1- | CASSLWAGNTIYF |
| | | | | | 3 | |
| A2:37 | TRAV14 | TRAJ5 | CAMIEWDTGRRALI | TRBV29-1 | TRBJ2- | CSVEDLGAGVSNEQFF |
| | | | F | | 1 | |
| A2:37 | TRAV38-1 | TRAJ31 | CAFFEYDNNARLMF | TRBV3-1 | TRBJ2- | CASSYEDHEQYF |
| | | | | | 7 | |
| A2:37 | TRAV38 | TRAJ57 | CAFLQGGSEKLVF | TRBV28 | TRBJ2- | CASSLFGLAGVEETQYF |
| | | | | | 5 | |
| A2:37 | TRAV38 | TRAJ28 | CAFMKHEDSGAGSY | TRBV3-1 | TRBJ2- | CASSQAISDREVWDQET |
| | | | QLTF | | 5 | QYF |
| A2:37 | TRAV19 | TRAJ12 | CALTEVDSSYKLIF | TRBV27 | TRBJ2- | CASSLAGGSYEQYF |
| | | | | | 7 | |
| A2:37 | TRAV17 | TRAJ5 | CAPSLMDTGRRALT | TRBV12-3 | TRBJ2- | CASSFGGDGYNEQFF |
| | | | F | | 1 | |
| A2:37 | TRAV9-2 | TRAJ29 | CASGLPDTPLVF | TRBV13 | TRBJ1- | CASSLRDRGNQPQHF |
| | | | | | 5 | |
| A2:37 | TRAV12- | TRAJ9 | CAMSAPDTGGFKTIF | | | |
| | 3 | | | | | |
| A2:37 | TRAV5 | TRAJ45 | CAESEGADGLTF | TRBV20-1 | TRBJ1- | CSASEGYTF |
| | | | | | 2 | |
| A2:37 | TRAV8-4 | TRAJ20 | CAVRNDYKLSF | TRBV6-3 | TRBJ2- | CASSYPTSGYNEQFF |
| | | | | | 1 | |
| A2:37 | TRAV21 | TRAJ24 | CAVITTDSWGKLQF | TRBV13 | TRBJ2- | CASSSGLAGASNEQFF |
| | | | | | 1 | |
| A2:37 | TRAV21 | TRAJ49 | CAVLGNQFYF | TRBV7-3 | TRBJ1- | CASSLVAGTDGYTF |
| | | | | | 2 | |
| A2:37 | TRAV13-2 | TRAJ24 | CADPTDSWGKLQF | TRBV14 | TRBJ2- | CASSLIGLAEQFF |
| | | | | | 1 | |
| A2:37 | TRAV12-1 | TRAJ20 | CVVIGPFNDYKLSF | TRBV29-1 | TRBJ1- | CSVEKGNNYGYTF |
| | | | | | 2 | |
| A2:37 | TRAV38-1 | TRAJ29 | CAFMKHEDTGGNT | TRBV7-6 | TRBJ2- | CASSLSQGIYYEQYF |
| | | | PLVF | | 7 | |
| A2:37 | TRAV12-2 | TRAJ56 | CAVKGAGANSKLTF | TRBV13 | TRBJ2- | CASRSDRVTEHTQYF |
| | | | | | 3 | |
| A2:37 | TRAV38- | TRAJ49 | CAYYPWNTGNQFYF | TRBV10-1 | TRBJ2- | CASSDGSYEQYF |
| | 2DV8 | | | | 7 | |
| A2:37 | TRAV21 | TRAJ26 | CAVSDYGQNFVF | TRBV10-2 | TRBJ1- | CASSDSGTEAFF |
| | | | | | 1 | |
| A2:37 | TRAV12-1 | TRAJ53 | CVGGGGSNYKLTF | TRBV27 | TRBJ2- | CASSLTPPGSYNEQFF |
| | | | | | 1 | |
| A2:37 | TRAV38-1 | TRAJ34 | CAFMKPDSGTYKYIF | TRBV7-6 | TRBJ2- | CASSRDPQPDTQYF |
| | | | | | 3 | |
| A2:37 | TRAV21 | TRAJ49 | CAVLGNQFYF | TRBV7-3 | TRBJ1- | CASSLVAGTDGYTF |
| | | | | | 2 | |
| A2:37 | TRAV38- | TRAJ48 | CAYRSPTALNEKLTF | TRBV28 | TRBJ2- | CASSLWTSGYETQYF |
| | 2DV8 | | | | 5 | |
| A2:37 | TRAV3 | TRAJ21 | CAVRAFGYNFNKFYF | TRBV28 | TRBJ2- | CASSLASGNYEQYF |
| | | | | | 7 | |
| A2:37 | TRAV12-2 | TRAJ30 | CAVSVRDDKIIF | TRBV5-6 | TRBJ2- | CASSFDRAEYEQYF |
| | | | | | 7 | |
| A2:37 | TRAV41 | TRAJ54 | CAPQGAQKLVF | TRBV27 | TRBJ2- | CASSLSAGAFSDTQYF |
| | | | | | 3 | |
| A2:37 | TRAV38- | TRAJ43 | CAYMNNNNDMRF | TRBV28 | TRBJ2- | CASSLPTQGGLIEQFF |
| | 2DV8 | | | | 1 | |
| A2:37 | TRAV19 | TRAJ31 | CALSEVDRLMF | TRBV28 | TRBJ2- | CASSLTGTDTQYF |
| | | | | | 3 | |
| A2:37 | TRAV13-2 | TRAJ15 | CAEMEGTALIF | TRBV4-1 | TRBJ1- | CASSQGAAEAFF |
| | | | | | 1 | |
| A2:37 | TRAV9-2 | TRAJ48 | CALSDPDMEKLTF | TRBV5-6 | TRBJ2- | CASSFGTPYEQYF |
| | | | | | 7 | |
| A2:37 | TRAV8-2 | TRAJ34 | CVVSFQGTDKLIF | TRBV20-1 | TRBJ2- | CSATGEAWTGWNEQFF |
| | | | | | 1 | |
| A2:37 | TRAV13-1 | TRAJ30 | CAAERDDKIIF | TRBV12-4 | TRBJ2- | CASSMTSGSPYNEQFF |
| | | | | | 1 | |
| A2:37 | TRAV3 | TRAJ28 | CAVRDSGAGSYQLT | TRBV12-4 | TRBJ1- | CATQDSLFMNTEAFF |
| | | | F | | 1 | |
| A2:37 | TRAV38- | TRAJ33 | CAYSNYQLIW | TRBV28 | TRBJ2- | CASSFVTSYEQYF |
| | 2DV8 | | | | 7 | |
| A2:37 | TRAV21 | TRAJ37 | CAVESGNTGKLIF | TRBV19 | TRBJ2- | CASSISSTGELFF |
| | | | | | 2 | |
| A2:37 | TRAV12-2 | TRAJ11 | CAGYPGYSTLTF | TRBV7-9 | TRBJ1- | CASSLGQYNSPLHF |
| | | | | | 6 | |
| A2:37 | TRAV8-4 | TRAJ20 | CAVRINYKLSF | TRBV6-2 | TRBJ2- | CASSRVTSGHNEQFF |
| | | | | | 1 | |
| A2:37 | TRAV3 | TRAJ30 | CAVRDGHRDDKIIF | TRBV7-9 | TRBJ2- | CASSLGVRAQKTQYF |
| | | | | | 5 | |
| A2:37 | TRAV38- | TRAJ9 | CAYNTGGFKTIF | TRBV28 | TRBJ2- | CATGPRGSSYNEQFF |
| | 2/DV8 | | | | 1 | |
| A2:37 | TRAV5 | TRAJ42 | CAESQGNLIF | TRBV14 | TRBJ2- | CASSQSPGGEQFF |
| | | | | | 1 | |
| A2:37 | TRAV38- | TRAJ49 | CAYRSPVSGNQFYF | TRBV28 | TRBJ1- | CASTPPRGPQHF |
| | 2/DV8 | | | | 5 | |
| A2:37 | TRAV27 | TRAJ44 | CAGGSGTASKLTF | TRBV27 | TRBJ2- | CASSVAGSYGDTQYF |
| | | | | | 3 | |
| A2:37 | TRAV27 | TRAJ54 | CAGAGEAGAQKLVF | TRBV27 | TRBJ2- | CASSPTGLVYEQFF |
| | | | | | 1 | |
| A2:37 | TRAV17 | TRAJ54 | CATDRDQGAQKLVF | TRBV4-2 | TRBJ2- | CASSQEVGIWQTQYF |
| | | | | | 5 | |
| A2:37 | TRAV8-6 | TRAJ33 | CTSNYQLIW | TRBV11-2 | TRBJ1- | CASSLFRETEAFF |
| | | | | | 1 | |
| A2:37 | TRAV12-2 | TRAJ49 | CAVSGGNQFYF | TRBV5-4 | TRBJ1- | CASSLTGETEKLFF |
| | | | | | 4 | |
| A2:37 | TRAV2 | TRAJ33 | CAVENYQLIW | TRBV18 | TRBJ2- | CASSQGQEKETQYF |
| | | | | | 5 | |
| A2:37 | TRAV14/ | TRAJ42 | CAMREGGSQGNLIF | TRBV19 | TRBJ1- | CASRFDGSNQPQHF |
| | DV4 | | | | 5 | |
| A2:37 | TRAV12-3 | TRAJ13 | CAMRGYQKVTF | TRBV3-1 | TRBJ2- | CASSHELTRADTQYF |
| | | | | | 3 | |
| A2:37 | TRAV1-2 | TRAJ28 | CAVRDSGAGSYQLT | TRBV5-6 | TRBJ2- | CASSAPVWEGTGELFF |
| | | | F | | 2 | |
| A2:37 | TRAV5 | TRAJ28 | CAEENSGAGSYQLTF | TRBV5-5 | TRBJ1- | CASSLGDSTEAFF |
| | | | | | 1 | |
| A2:37 | TRAV5 | TRAJ13 | CAESMSYQKVTF | TRBV5-1 | TRBJ2- | CASSLEASTDTQYF |
| | | | | | 3 | |
| A2:37 | TRAV26-2 | TRAJ43 | CILDNNNDMRF | TRBV7-6 | TRBJ1- | CASSLAPGTTNEKLFF |
| | | | | | 4 | |
| A2:37 | TRAV8-1 | TRAJ33 | CAVNAVDSNYQLIW | TRBV28 | TRBJ1- | CASSGFGKLFF |
| | | | | | 4 | |
| A2:37 | TRAV19 | TRAJ40 | CALSEANEGTYKYIF | TRBV27 | TRBJ2- | CASSNSIGSADTDTQYF |
| | | | | | 3 | |
| A2:37 | TRAV29 | TRAJ47 | CAASDGGNKLVF | TRBV12-4 | TRBJ2- | CASMGGLAGGYADTQY |
| | | | | | 3 | F |
| A2:37 | TRAV8-6 | TRAJ43 | CAVSPYNNNDMRF | TRBV12-3 | TRBJ2- | CASRPLAAQETQYF |
| | | | | | 5 | |
| A2:37 | TRAV5 | TRAJ12 | CAEYAMDSSYKLIF | TRBV2 | TRBJ1- | CASSEGEGFYGYTF |
| | | | | | 4 | |
| A2:37 | TRAV38- | TRAJ30 | CAYRTPLRDDKIIF | TRBV28 | TRBJ2- | CASSDTAGSSYNEQFF |
| | 2/DV8 | | | | 1 | |
| A2:37 | TRAV41 | TRAJ56 | CAADTNYYTGANSK | TRBV27 | TRBJ2- | CASSFGRDLNTGELFF |
| | | | LTF | | 2 | |
| A2:37 | TRAV21 | TRAJ48 | CAVKGFFGNEKLTF | | | |
| A2:37 | TRAV1-2 | TRAJ31 | CAVRDNNARLMF | TRBV10-3 | TRBJ1- | CAIDPTGSLNQPQHF |
| | | | | | 5 | |
| A2:37 | TRAV8-6 | TRAJ8 | CAVSDPDTGFQKLV | TRBV14 | TRBJ2- | CASSRQQGVEQYV |
| | | | F | | 7 | |
| A2:37 | TRAV22 | TRAJ43 | CAVDITWNDMRF | TRBV28 | TRBJ2- | CATQNNEQFF |
| | | | | | 1 | |
| A2:37 | TRAV22 | TRAJ57 | CAVPLRADLTKLVF | TRBV27 | TRBJ2- | CASSLEVGLAPNEQFF |
| | | | | | 1 | |
| A2:37 | TRAV9-2 | TRAJ52 | CALSDRDGGTSYGKL | TRBV6-5 | TRBJ2- | CASSYSPGYEQYF |
| | | | TF | | 7 | |
| A2:37 | TRAV19 | TRAJ40 | CALSEATSGTYKYIF | TRBV27 | TRBJ1- | CASSLITGDTEAFF |
| | | | | | 1 | |
| A2:47 | TRAV14 | TRAJ48 | CAMRAFGNEKLTF | TRBV7-9 | TRBJ2- | CASSPRDEQFF |
| | | | | | 1 | |

SF3B1^{mut}-Induced Neo-Epitopes on Tumor Cells are Recognized by Specific CD8 T Cells.

To determine whether the SF3B1-induced neo-epitopes were presented on the surface of the tumor cells themselves, the inventors generated tools to analyze the direct interaction between the neo-epitope specific CD8 T cells and tumor cells. The available SF2B1^{mut} UM cell line (Mel-202) being HLA-A2^{neg}, it was transduced with an HLA-A2 expression vector. They also generated an isogenic negative control by inserting a DEGRON sequence in the SF3B1-mutated allele using Crispr-Cas9 technology, fully normalizing the splicing pattern (15). The resulting SF3B1^{wt} cell line was also transfected with HLA-A2. In parallel, we generated T cell clones for 4 SF3B1^{mut} neo-epitopes (A2:14, :18, :26 and:37) by direct FACS assisted single cell cloning of tetramer⁺ CD8⁺ T cells from both healthy donors (HD) and UM patients, followed by expansion and verification of the specificity using the relevant tetramers. 20 clones were obtained. The T cell clone functionality was verified by stimulating them with HLA-A2 transfected K562 loaded with the relevant peptide followed by assessment of interferon gamma (IFN-γ, granzyme B (GzmB) and IL-6 release. For each specificity, the clones with the highest sensitivity (activated by the lowest peptide concentration) were selected for the following functional assays.

SF3B1^{mut} and SF3B1^{wt} HLA-A2⁺ or HLA-A2⁻ Mel-202 UM cell lines were used to stimulate the T cell clones. An A2:18 specific T cell clone from an HD was specifically activated by SF3B1^{mut} and not by SF3B1^{wt} Mel-202 UM cells as seen by CD25 upregulation (FIG. 6A) and GzmB secretion after a 24 h incubation (FIG. 6B). Clones specific for A2:26 and A2:37 from patient UM2 secreted more lymphokines after stimulation by SF3B1^{mut} cells in comparison with SF3B1^{wt} Mel-202 UM cells in an HLA-A2 dependent manner (FIG. 6C, D). Similarly, an A2:14-specific T cell clone from an HD was activated by a SF3B1^{mut} but not SF3B1^{wt} HLA-A2⁺ UM PDX as evidenced by CD25 (FIG. 6E) upregulation. This SF3B1^{mut} PDX also activated an A2:18 specific T cell clone from an HD (FIG. 6F, G) as well as an A2:37-specific T cell clone from patient UM1 (FIG. 6H). These results demonstrate that the four SF3B1^{mut} neo-epitopes for which a memory T cell response was observed in the peripheral blood of UM patients can be directly recognized on tumor cells by CD8 T cells.

Importantly, a T cell clone specific for neo-epitope A2:18 killed SF3B1^{mut} HLA-A2⁺ Mel-202 UM cells and not the SF3B1^{wt} isogenic cells, while an irrelevant A2-CMV clone did not (FIG. 6I). T cell clones for the other specificities (A2:18, :26 and :37) also specifically killed SF3B1^{mut} HLA-A2⁺ Mel-202 UM cells and not the SF3B1^{wt} isogenic cells (FIG. 6J). Thus, SF3B1^{mut} related neo-epitopes stimulate the immune system of UM patients at the metastatic stage to generate circulating CD8⁺ T cells that are specific for the tumor neo-epitopes and can directly recognize and kill tumor cells.

Concluding Remarks

In this work, the inventors show that mutations in the splicing factor SF3B1 in UM tumors generate MHC class I restricted tumor neo-epitopes that are detected by patients' CD8 T cells. Expanded T cells specific for these antigens are enriched at the tumor site. These neo-epitopes are expressed by tumor cells and can be directly recognized by the specific CD8 T cells able to kill the SF3B1^{mut} UM cells.

To their knowledge, this work is the first experimental demonstration of an immune response against public neo-epitopes generated by an abnormal splicing process, an hypothesis that was suggested some time ago (5, 16). Here, they identified and demonstrated a relevant immune response towards MHC class I restricted epitopes. It is probable that MHC-II restricted epitopes are also generated and presented by antigen presenting cells to induce neo-epitope specific CD4⁺ T cells that may correspond to the chronically activated CD4⁺ T cells they previously described in cancer patients (17).

Since the splicing factor SF3B1 is only mutated in the tumor cells and modifies the splicing pattern in over 1000 junctions, the neo epitopes are tumor-specific and numerous. Being germline encoded, these neo-epitopes are shared across most patients according to their particular HLA haplotype. HLA-A2 is expressed by ˜45% of the individuals in Europe. By characterizing SF3B1^{mut}-related epitopes presented by other prevalent HLA alleles, it can be envisioned that a limited (15-20) number of public neo-epitopes would enable treatment of almost all patients. Efficient vaccination using long peptides, DNA- or RNA-based vaccines would be relatively easy to manufacture (2). Neo-epitopes are also attractive targets for adoptive transfer therapies relying either on T cells transduced with specific TCRs or on soluble bi-specific reagents redirecting the activity of effector T cells towards neo-epitopes expressing tumor cells with antibodies or affinity matured TCR, similarly to what is proposed for the Melan-A HLA-A2 epitope (18). Notably, SF3B1 mutations are not restricted to uveal melanoma, but also found in a wide range of malignancies (7), including hemopathies (19, 20), carcinomas and other melanomas(21), in which a significant proportion of abnormal junctions are shared with UM (11), further extending the potential therapeutic importance of our finding.
1. N. A. Rizvi et al., Cancer immunology. Mutational landscape determines sensitivity to PD-1 blockade in non-small cell lung cancer. Science 348, 124-128 (2015).
2. U. Sahin, O. Tureci, Personalized vaccines for cancer immunotherapy. Science 359, 1355-1360 (2018).
3. P. G. Coulie, B. J. Van den Eynde, P. van der Bruggen, T. Boon, Tumour antigens recognized by T lymphocytes: at the core of cancer immunotherapy. Nat Rev Cancer 14, 135-146 (2014).
4. J. Gotter, B. Brors, M. Hergenhahn, B. Kyewski, Medullary epithelial cells of the human thymus express a highly diverse selection of tissue-specific genes colocalized in chromosomal clusters. J Exp Med 199, 155-166 (2004).
5. J. E. Slansky, P. T. Spellman, Alternative Splicing in Tumors—A Path to Immunogenicity? N Engl J Med 380, 877-880 (2019).
6. E. A. Obeng, C. Stewart, O. Abdel-Wahab, Altered RNA Processing in Cancer Pathogenesis and Therapy. Cancer discovery 9, 1493-1510 (2019).
7. R. F. Wang, H. Y. Wang, Immune targets and neoantigens for cancer immunotherapy and precision medicine. Cell Res 27, 11-37 (2017).
8. M. Rodrigues et al., So Close, yet so Far: Discrepancies between Uveal and Other Melanomas. A Position Paper from UM Cure 2020. Cancers (Basel) 11, (2019).
9. M. Rodrigues et al., Evolutionary Routes in Metastatic Uveal Melanomas Depend on MBD4 Alterations. Clin Cancer Res 25, 5513-5524 (2019).
10. S. Alsafadi et al., Cancer-associated SF3B1 mutations affect alternative splicing by promoting alternative branchpoint usage. Nature communications 7, 10615 (2016).
11. R. B. Darman et al., Cancer-Associated SF3B1 Hotspot Mutations Induce Cryptic 3′ Splice Site Selection through Use of a Different Branch Point. Cell Rep 13, 1033-1045 (2015).
12. V. Jurtz et al., NetMHCpan-4.0: Improved Peptide-MHC Class I Interaction Predictions Integrating Eluted Ligand and Peptide Binding Affinity Data. J Immunol 199, 3360-3368 (2017).
13. C. Leisner et al., One-pot, mix-and-read peptide-MHC tetramers. PloS one 3, e1678 (2008).
14. S. D'Souza et al., Circulating Melan-A/Mart-1 specific cytolytic T lymphocyte precursors in HLA-A2+ melanoma patients have a memory phenotype. Int J Cancer 78, 699-706 (1998).
15. Q. Zhou et al., A chemical genetics approach for the functional assessment of novel cancer genes. Cancer research 75, 1949-1958 (2015).
16. L. Frankiw, D. Baltimore, G. Li, Alternative mRNA splicing in cancer immunotherapy. Nat Rev Immunol 19, 675-687 (2019).
17. I. Peguillet et al., High numbers of differentiated effector CD4 T cells are found in patients with cancer and correlate with clinical response after neoadjuvant therapy of breast cancer. Cancer research 74, 2204-2216 (2014).
18. N. Liddy et al., Monoclonal TCR-redirected tumor cell killing. Nat Med 18, 980-987 (2012).
19. K. Yoshida et al., Frequent pathway mutations of splicing machinery in myelodysplasia. Nature 478, 64-69 (2011).
20. L. Wang et al., SF3B1 and other novel cancer genes in chronic lymphocytic leukemia. N Engl J Med 365, 2497-2506 (2011).
21. A. Kahles et al., Comprehensive Analysis of Alternative Splicing Across Tumors from 8,705 Patients. Cancer Cell 34, 211-224 e216 (2018).
22. F. Nemati et al., Establishment and characterization of a panel of human uveal melanoma xenografts derived from primary and/or metastatic tumors. Clin Cancer Res 16, 2352-2362 (2010).
23. K. Wang, M. Li, H. Hakonarson, ANNOVAR: functional annotation of genetic variants from high-throughput sequencing data. Nucleic Acids Res 38, e164 (2010).
24. B. Rodenko et al., Generation of peptide-MHC class I complexes through UV-mediated ligand exchange. Nat Protoc 1, 1120-1132 (2006).
25. A. Lissina et al., Protein kinase inhibitors substantially improve the physical detection of T-cells with peptide-MHC tetramers. J Immunol Methods 340, 11-24 (2009).
26. S. R. Hadrup, T. N. Schumacher, MHC-based detection of antigen-specific CD8+ T cell responses. Cancer Immunol Immunother 59, 1425-1433 (2010).
27. A. Han, J. Glanville, L. Hansmann, M. M. Davis, Linking T-cell receptor sequence to functional phenotype at the single-cell level. Nature biotechnology 32, 684-692 (2014).
28. V. Giudicelli, D. Chaume, M. P. Lefranc, IMGT/GENE-DB: a comprehensive database for human and mouse immunoglobulin and T cell receptor genes. Nucleic Acids Res 33, D256-261 (2005).
29. S. Le Gall et al., Nef interacts with the mu subunit of clathrin adaptor complexes and reveals a cryptic sorting signal in MHC I molecules. Immunity 8, 483-495 (1998).
30. C. Zheng et al., Landscape of Infiltrating T Cells in Liver Cancer Revealed by Single-Cell Sequencing. Cell 169, 1342-1356 e1316 (2017).
31. A. Butler, P. Hoffman, P. Smibert, E. Papalexi, R. Satija, Integrating single-cell transcriptomic data across different conditions, technologies, and species. Nature biotechnology 36, 411-420 (2018).
32. L. Zappia, A. Oshlack, Clustering trees: a visualization for evaluating clusterings at multiple resolutions. Gigascience 7, (2018).

### SEQUENCE LISTING

| | |
|---|---|
| SEQ ID NO: 1-1058 | Neoantigenic peptides that bind with high |
| | stringency (rank < 0; 5% see methods) on at |
| | least one HLA allele and wherein frequency |
| | in a human population (n > 5000) is at least |
| | of 1% |
| SEQ ID NO: 1059-1148 | CDR3 TCR alpha AA |
| SEQ ID NO: 1149-1233 | CDR3 TCR beta AA |

1-17. (canceled)

18. A tumor specific neoantigenic peptide, wherein said peptide
(a) is encoded by a part of an ORF sequence from a transcript associated with a SF3B1 or a SF3B1-like mutation, present in a SF3B1 mutant tumor sample;
(b) comprises at least 8 amino acids and binds at least one MHC molecule with an affinity of less than 500 nM; and
(c) is not expressed in normal healthy cells; or is any of SEQ ID NO: 1-1058.

19. The tumor specific neoantigenic peptide according to claim 18, wherein said peptide is 8 or 9 amino acids long, and binds at least one MHC class I molecule.

20. The tumor specific neoantigenic peptide according to claim 18, wherein the SF3B1 mutant tumor is selected from uveal melanoma, hematological malignancies, breast cancers, skin melanoma, renal cell carcinoma, pulmonary adenocarcinoma, hepatocarcinoma, pancreatic carcinoma, endometrial cancers and uveal melanoma.

21. The tumor specific neoantigenic peptide according to claim 18, which is encoded by a part of an ORF sequence from a transcript associated with a SF3B1 or SUGP1 mutation.

22. A population of autologous dendritic cells or antigen presenting cells that have been pulsed with one or more or two or more neoantigenic peptides according to claim 18 or transfected with a polynucleotide encoding one or more or two or more neoantigenic peptides according to claim 18.

23. A vaccine or immunogenic composition capable of rising a specific T-cell response comprising:
(a) one or more neoantigenic peptides according to claim 18;
(b) one or more polynucleotides encoding one or more neoantigenic peptides according to claim 18;
(c) one or more of said polynucleotides in b., further linked to a heterologous regulatory control nucleotide sequence;
(d) a population of autologous dendritic cells or antigen presenting cells that have been pulsed with one or more or two or more neoantigenic peptides according to claim 18, or transfected with a polynucleotide encoding one or more or two or more neoantigenic peptides according to claim 18;
(e) one or more recombinant MHC molecules loaded with neoantigenic peptides according to claim 18; and/or
(f) one or more of said neoantigenic peptides in (a), said polynucleotides in (b) or (c), said recombinant MHC molecules in e., or said population of autologous dendritic cells or antigen presenting cells in (d), wherein the neoantigenic peptides comprise at least one peptide which is encoded by a canonical ORF and/or at least one which is encoded by a non-canonical ORF.

24. An antibody, or an antigen-binding fragment thereof, a T cell receptor (TCR), or a chimeric antigen receptor (CAR) that specifically binds a neoantigenic peptide according to claim 18, in association with an MHC molecule, with a Kd affinity of about 10⁻⁶ M or less.

25. A T cell receptor that specifically binds a neoantigenic peptide according to claim 18, in association with an MHC molecule, with a Kd affinity of about 10⁻⁶ M or less, wherein said T cell receptor is made soluble and fused to an antibody fragment directed to a T cell antigen.

26. The T cell receptor according to claim 25, wherein the targeted antigen is CD3 or CD16.

27. An antibody that specifically binds a neoantigenic peptide according to claim 18, in association with an MHC molecule, with a Kd affinity of about 10⁻⁶ M or less, wherein said antibody is a multi-specific antibody that further targets at least an immune cell antigen.

28. The antibody according to claim 27, wherein the immune cell is a T cell, a NK cell or a dendritic cell, and/or wherein the targeted antigen is CD3, CD16, CD30 or a TCR.

29. A polynucleotide encoding a neoantigenic peptide according to claim 18, or an antibody, a CAR or a TCR that specifically binds a neoantigenic peptide according to claim 18, in association with an MHC molecule with a Kd affinity of about 10⁻⁶ M or less, or a vector comprising the polynucleotide.

30. An immune cell that specifically binds to one or more neoantigenic peptides according to claim 18.

31. The immune cell according to claim 30, which is an allogenic or autologous cell selected from T cell, NK cell, CD4+/CD8+, TILs/tumor derived CD8 T cells, central memory CD8+ T cells, Treg, MAIT, and γδ T cell.

32. An immune cell which is a T cell comprising:
(a) a T cell receptor that specifically binds one or more neoantigenic peptides according to claim 18, or
(b) a TCR or a CAR that specifically binds a neoantigenic peptide according to claim 18, in association with an MHC molecule, with a Kd affinity of about 10⁻⁶ M or less.

33. A method of cancer vaccination therapy, comprising administering to a subject in need thereof, a therapeutically effective amount of:
(i) a neoantigenic peptide according to claim 18;
(ii) a population of dendritic cells or antigen presenting cells that have been pulsed with one or more or two or more neoantigenic peptides according to claim 18 or transfected with a polynucleotide encoding one or more or two or more neoantigenic peptides according to claim 18;
(iii) one or more recombinant MHC molecules loaded with neoantigenic peptides according to claim 18; or
(iv) a polynucleotide encoding one or more neoantigenic peptides according to claim 18 or a vector comprising the polynucleotide.

34. The method according to claim 33, wherein the subject is suffering from an SF3B1 mutant associated uveal melanoma or is at risk of suffering from an SF3B1 mutant associated uveal melanoma.

35. The method according to claim 33, wherein the therapy is administered in combination with a chemotherapeutic agent or an immunotherapeutic agent.

36. A method for inhibiting cancer cell proliferation, or for the treatment of cancer in a subject in need thereof, comprising administering to the subject, a therapeutically effective amount of:
(i) a neoantigenic peptide according to claim 18;
(ii) a population of dendritic cells or antigen presenting cells that have been pulsed with one or more or two or more neoantigenic peptides according to claim 18 or transfected with a polynucleotide encoding one or more or two or more neoantigenic peptides according to claim 18;
(iii) a polynucleotide encoding one or more neoantigenic peptides according to claim 18 or a vector comprising the polynucleotide;
(iv) an antibody, or an antigen-binding fragment thereof, a T cell receptor (TCR), or a chimeric antigen receptor (CAR) that specifically binds a neoantigenic peptide according to claim 18, in association with an MHC molecule, with a Kd affinity of about 10⁻⁶ M or less; or
(v) an immune cell that specifically binds to one or more neoantigenic peptides according to claim 18.

37. The method according to claim 36, wherein the subject is suffering from an SF3B1 mutant associated uveal melanoma or is at risk of suffering from an SF3B1 mutant associated uveal melanoma.

38. The method according to claim 36, wherein the treatment is administered in combination with a chemotherapeutic agent or an immunotherapeutic agent.
